SEQUENCE LISTING (288 sequences)

SEQ ID NO: 1 - protein, 7 aa, Artificial Sequence
  peptide
LRRNPAD

SEQ ID NO: 2 - protein, 7 aa, Artificial Sequence
  peptide
NPADYRG

SEQ ID NO: 3 - protein, 7 aa, Artificial Sequence
  peptide
NPADYHG

SEQ ID NO: 4 - protein, 11 aa, Artificial Sequence
  peptide
VRLRWNPADYP

SEQ ID NO: 5 - protein, 7 aa, Artificial Sequence
  peptide
LRGNPAD

SEQ ID NO: 6 - protein, 7 aa, Artificial Sequence
  peptide
WNPADYR

SEQ ID NO: 7 - protein, 7 aa, Artificial Sequence
  peptide
LRFNPAD

SEQ ID NO: 8 - protein, 7 aa, Artificial Sequence
  peptide
GSLRYNP

SEQ ID NO: 9 - protein, 9 aa, Artificial Sequence
  peptide
LRVNPADYG

SEQ ID NO: 10 - protein, 9 aa, Artificial Sequence
  peptide
LRRNPADYG

SEQ ID NO: 11 - protein, 7 aa, Artificial Sequence
  peptide
VRLRFNP

SEQ ID NO: 12 - protein, 7 aa, Artificial Sequence
  peptide
VRLRYNP

SEQ ID NO: 13 - protein, 7 aa, Artificial Sequence
  peptide
LRLNPAD

SEQ ID NO: 14 - protein, 9 aa, Artificial Sequence
  peptide
VRLRRNPAD

SEQ ID NO: 15 - protein, 7 aa, Artificial Sequence
  peptide
RFNPADY

SEQ ID NO: 16 - protein, 7 aa, Artificial Sequence
  peptide
GSLRFNP

SEQ ID NO: 17 - protein, 7 aa, Artificial Sequence
  peptide
NPDDYHG

SEQ ID NO: 18 - protein, 7 aa, Artificial Sequence
  peptide
NPADYGY

SEQ ID NO: 19 - protein, 7 aa, Artificial Sequence
  peptide
WNPADYP

SEQ ID NO: 20 - protein, 9 aa, Artificial Sequence
  peptide
RLHWNPADY

SEQ ID NO: 21 - protein, 11 aa, Artificial Sequence
  peptide
QWIDVRLRFNP

SEQ ID NO: 22 - protein, 7 aa, Artificial Sequence
  peptide
NPADYPG

SEQ ID NO: 23 - protein, 7 aa, Artificial Sequence
  peptide
NPADYGH

SEQ ID NO: 24 - protein, 7 aa, Artificial Sequence
  peptide
RLNPADY

SEQ ID NO: 25 - protein, 9 aa, Artificial Sequence
  peptide
LRLNPADYG

SEQ ID NO: 26 - protein, 11 aa, Artificial Sequence
  peptide
LLGGGSLRFNP

SEQ ID NO: 27 - protein, 7 aa, Artificial Sequence
  peptide
RYNPADY

SEQ ID NO: 28 - protein, 7 aa, Artificial Sequence
  peptide
LRYNPAD

SEQ ID NO: 29 - protein, 7 aa, Artificial Sequence
  peptide
LHWNPAD

SEQ ID NO: 30 - protein, 9 aa, Artificial Sequence
  peptide
LRWNPADYH

SEQ ID NO: 31 - protein, 9 aa, Artificial Sequence
  peptide
IDVRLRFNP

SEQ ID NO: 32 - protein, 9 aa, Artificial Sequence
  peptide
RLRVNPADY

SEQ ID NO: 33 - protein, 7 aa, Artificial Sequence
  peptide
NPADYGD

SEQ ID NO: 34 - protein, 9 aa, Artificial Sequence
  peptide
WNPADYGHI

SEQ ID NO: 35 - protein, 7 aa, Artificial Sequence
  peptide
YNLKFNP

SEQ ID NO: 36 - protein, 9 aa, Artificial Sequence
  peptide
SLHWNPADY

SEQ ID NO: 37 - protein, 11 aa, Artificial Sequence
  peptide
QWIDVRLRYNP

SEQ ID NO: 38 - protein, 9 aa, Artificial Sequence
  peptide
RGNPADYGG

SEQ ID NO: 39 - protein, 9 aa, Artificial Sequence
  peptide
GGGSLRFNP

SEQ ID NO: 40 - protein, 9 aa, Artificial Sequence
  peptide
LRINPADYG

SEQ ID NO: 41 - protein, 7 aa, Artificial Sequence
  peptide
RWNPADF

SEQ ID NO: 42 - protein, 11 aa, Artificial Sequence
  peptide
RLRWNPADYHG

SEQ ID NO: 43 - protein, 9 aa, Artificial Sequence
  peptide
IDVRLRYNP

SEQ ID NO: 44 - protein, 7 aa, Artificial Sequence
  peptide
RINPADY

SEQ ID NO: 45 - protein, 7 aa, Artificial Sequence
  peptide
NPADYGR

SEQ ID NO: 46 - protein, 9 aa, Artificial Sequence
  peptide
RLRFNPADY

SEQ ID NO: 47 - protein, 7 aa, Artificial Sequence
  peptide
WNPDDYR

SEQ ID NO: 48 - protein, 7 aa, Artificial Sequence
  peptide
CNLKFNP

SEQ ID NO: 49 - protein, 9 aa, Artificial Sequence
  peptide
SLRINPADY

SEQ ID NO: 50 - protein, 9 aa, Artificial Sequence
  peptide
RLYWNPADY

SEQ ID NO: 51 - protein, 11 aa, Artificial Sequence
  peptide
QWVDYNLKFNP

SEQ ID NO: 52 - protein, 11 aa, Artificial Sequence
  peptide
GGGSLRRNPAD

SEQ ID NO: 53 - protein, 11 aa, Artificial Sequence
  peptide
LRWNPADYGGF

SEQ ID NO: 54 - protein, 11 aa, Artificial Sequence
  peptide
SLRWNPADYHG

SEQ ID NO: 55 - protein, 7 aa, Artificial Sequence
  peptide
NPADYDG

SEQ ID NO: 56 - protein, 11 aa, Artificial Sequence
  peptide
DVRLRFNPADY

SEQ ID NO: 57 - protein, 7 aa, Artificial Sequence
  peptide
RRNPADY

SEQ ID NO: 58 - protein, 7 aa, Artificial Sequence
  peptide
RPNPADY

SEQ ID NO: 59 - protein, 9 aa, Artificial Sequence
  peptide
GSLRRNPAD

SEQ ID NO: 60 - protein, 7 aa, Artificial Sequence
  peptide
WNPADYH

SEQ ID NO: 61 - protein, 9 aa, Artificial Sequence
  peptide
RWNPADYGH

SEQ ID NO: 62 - protein, 9 aa, Artificial Sequence
  peptide
GSLRFNPAD

SEQ ID NO: 63 - protein, 7 aa, Artificial Sequence
  peptide
LRINPAD

SEQ ID NO: 64 - protein, 9 aa, Artificial Sequence
  peptide
WRLRWNPAD

SEQ ID NO: 65 - protein, 9 aa, Artificial Sequence
  peptide
GNPADYGGI

SEQ ID NO: 66 - protein, 7 aa, Artificial Sequence
  peptide
RHNPADY

SEQ ID NO: 67 - protein, 9 aa, Artificial Sequence
  peptide
WNPDDYGHV

SEQ ID NO: 68 - protein, 9 aa, Artificial Sequence
  peptide
RPNPADYGG

SEQ ID NO: 69 - protein, 11 aa, Artificial Sequence
  peptide
DVRLRINPADY

SEQ ID NO: 70 - protein, 7 aa, Artificial Sequence
  peptide
YNLKYNP

SEQ ID NO: 71 - protein, 11 aa, Artificial Sequence
  peptide
GGGSLRFNPAD

SEQ ID NO: 72 - protein, 9 aa, Artificial Sequence
  peptide
VRHRWNPAD

SEQ ID NO: 73 - protein, 7 aa, Artificial Sequence
  peptide
NPADYGI

SEQ ID NO: 74 - protein, 11 aa, Artificial Sequence
  peptide
IDVRLRFNPAD

SEQ ID NO: 75 - protein, 7 aa, Artificial Sequence
  peptide
NPDDYGH

SEQ ID NO: 76 - protein, 9 aa, Artificial Sequence
  peptide
VPLRWNPAD

SEQ ID NO: 77 - protein, 7 aa, Artificial Sequence
  peptide
YNLPWNP

SEQ ID NO: 78 - protein, 9 aa, Artificial Sequence
  peptide
VDYNLKFNP

SEQ ID NO: 79 - protein, 7 aa, Artificial Sequence
  peptide
HWNPADY

SEQ ID NO: 80 - protein, 7 aa, Artificial Sequence
  peptide
RTNPADY

SEQ ID NO: 81 - protein, 11 aa, Artificial Sequence
  peptide
LRWNPADYHGI

SEQ ID NO: 82 - protein, 9 aa, Artificial Sequence
  peptide
GGGSLRYNP

SEQ ID NO: 83 - protein, 9 aa, Artificial Sequence
  peptide
RLRGNPADY

SEQ ID NO: 84 - protein, 7 aa, Artificial Sequence
  peptide
RGNPADY

SEQ ID NO: 85 - protein, 9 aa, Artificial Sequence
  peptide
VDCNLKFNP

SEQ ID NO: 86 - protein, 11 aa, Artificial Sequence
  peptide
DVRLRWNPSDY

SEQ ID NO: 87 - protein, 9 aa, Artificial Sequence
  peptide
RIRWNPADY

SEQ ID NO: 88 - protein, 9 aa, Artificial Sequence
  peptide
HRLRWNPAD

SEQ ID NO: 89 - protein, 9 aa, Artificial Sequence
  peptide
SLRVNPADY

SEQ ID NO: 90 - protein, 7 aa, Artificial Sequence
  peptide
CNLKYNP

SEQ ID NO: 91 - protein, 9 aa, Artificial Sequence
  peptide
HSLRWNPAD

SEQ ID NO: 92 - protein, 7 aa, Artificial Sequence
  peptide
NPADYGN

SEQ ID NO: 93 - protein, 11 aa, Artificial Sequence
  peptide
LRWNPADYPGI

SEQ ID NO: 94 - protein, 9 aa, Artificial Sequence
  peptide
RLRYNPADY

SEQ ID NO: 95 - protein, 9 aa, Artificial Sequence
  peptide
SLRFNPADY

SEQ ID NO: 96 - protein, 11 aa, Artificial Sequence
  peptide
QWVDCNLKFNP

SEQ ID NO: 97 - protein, 9 aa, Artificial Sequence
  peptide
LRPNPADYG

SEQ ID NO: 98 - protein, 9 aa, Artificial Sequence
  peptide
VDCNLKYNP

SEQ ID NO: 99 - protein, 9 aa, Artificial Sequence
  peptide
ALRWNPADY

SEQ ID NO: 100 - protein, 7 aa, Artificial Sequence
  peptide
NPADYGV

SEQ ID NO: 101 - protein, 11 aa, Artificial Sequence
  peptide
VRLRWNPADYP

SEQ ID NO: 102 - protein, 9 aa, Artificial Sequence
  peptide
RLRVNPADY

SEQ ID NO: 103 - protein, 9 aa, Artificial Sequence
  peptide
LRVNPADYG

SEQ ID NO: 104 - protein, 11 aa, Artificial Sequence
  peptide
WIDVRLRGNPA

SEQ ID NO: 105 - protein, 7 aa, Artificial Sequence
  peptide
RLNPADY

SEQ ID NO: 106 - protein, 7 aa, Artificial Sequence
  peptide
RFNPADY

SEQ ID NO: 107 - protein, 9 aa, Artificial Sequence
  peptide
RLRLNPADY

SEQ ID NO: 108 - protein, 9 aa, Artificial Sequence
  peptide
RLRGNPADY

SEQ ID NO: 109 - protein, 11 aa, Artificial Sequence
  peptide
DVRLRINPADY

SEQ ID NO: 110 - protein, 11 aa, Artificial Sequence
  peptide
DVRLRVNPADY

SEQ ID NO: 111 - protein, 7 aa, Artificial Sequence
  peptide
RINPADY

SEQ ID NO: 112 - protein, 9 aa, Artificial Sequence
  peptide
LRLNPADYG

SEQ ID NO: 113 - protein, 7 aa, Artificial Sequence
  peptide
LRRNPAD

SEQ ID NO: 114 - protein, 9 aa, Artificial Sequence
  peptide
SLRINPADY

SEQ ID NO: 115 - protein, 11 aa, Artificial Sequence
  peptide
DVRLRFNPADY

SEQ ID NO: 116 - protein, 7 aa, Artificial Sequence
  peptide
NPADYRG

SEQ ID NO: 117 - protein, 9 aa, Artificial Sequence
  peptide
SLRVNPADY

SEQ ID NO: 118 - protein, 9 aa, Artificial Sequence
  peptide
RLRINPADY

SEQ ID NO: 119 - protein, 11 aa, Artificial Sequence
  peptide
GGSLRINPADY

SEQ ID NO: 120 - protein, 11 aa, Artificial Sequence
  peptide
DVRLRGNPADY

SEQ ID NO: 121 - protein, 7 aa, Artificial Sequence
  peptide
WNPADYR

SEQ ID NO: 122 - protein, 9 aa, Artificial Sequence
  peptide
RLRYNPADY

SEQ ID NO: 123 - protein, 7 aa, Artificial Sequence
  peptide
RGNPADY

SEQ ID NO: 124 - protein, 7 aa, Artificial Sequence
  peptide
RRNPADY

SEQ ID NO: 125 - protein, 9 aa, Artificial Sequence
  peptide
LRRNPADYG

SEQ ID NO: 126 - protein, 9 aa, Artificial Sequence
  peptide
RINPADYGG

SEQ ID NO: 127 - protein, 9 aa, Artificial Sequence
  peptide
LRINPADYG

SEQ ID NO: 128 - protein, 11 aa, Artificial Sequence
  peptide
GGSLRVNPADY

SEQ ID NO: 129 - protein, 9 aa, Artificial Sequence
  peptide
RGNPADYGG

SEQ ID NO: 130 - protein, 9 aa, Artificial Sequence
  peptide
SLRLNPADY

SEQ ID NO: 131 - protein, 11 aa, Artificial Sequence
  peptide
DVRLRPNPADY

SEQ ID NO: 132 - protein, 9 aa, Artificial Sequence
  peptide
RLRFNPADY

SEQ ID NO: 133 - protein, 7 aa, Artificial Sequence
  peptide
WNPADYP

SEQ ID NO: 134 - protein, 7 aa, Artificial Sequence
  peptide
RYNPADY

SEQ ID NO: 135 - protein, 11 aa, Artificial Sequence
  peptide
GGSLRGNPADY

SEQ ID NO: 136 - protein, 7 aa, Artificial Sequence
  peptide
NPADYGR

SEQ ID NO: 137 - protein, 7 aa, Artificial Sequence
  peptide
RVNPADY

SEQ ID NO: 138 - protein, 11 aa, Artificial Sequence
  peptide
GSLRWNPADYR

SEQ ID NO: 139 - protein, 7 aa, Artificial Sequence
  peptide
NPADYGH

SEQ ID NO: 140 - protein, 7 aa, Artificial Sequence
  peptide
RPNPADY

SEQ ID NO: 141 - protein, 9 aa, Artificial Sequence
  peptide
SLRGNPADY

SEQ ID NO: 142 - protein, 9 aa, Artificial Sequence
  peptide
VRLRRNPAD

SEQ ID NO: 143 - protein, 7 aa, Artificial Sequence
  peptide
RTNPADY

SEQ ID NO: 144 - protein, 11 aa, Artificial Sequence
  peptide
GGSLRFNPADY

SEQ ID NO: 145 - protein, 9 aa, Artificial Sequence
  peptide
RLYWNPADY

SEQ ID NO: 146 - protein, 9 aa, Artificial Sequence
  peptide
GNPADYGGI

SEQ ID NO: 147 - protein, 9 aa, Artificial Sequence
  peptide
SLRFNPADY

SEQ ID NO: 148 - protein, 11 aa, Artificial Sequence
  peptide
VRLRVNPADYG

SEQ ID NO: 149 - protein, 9 aa, Artificial Sequence
  peptide
LRWNPADYP

SEQ ID NO: 150 - protein, 7 aa, Artificial Sequence
  peptide
NPADYGD

SEQ ID NO: 151 - protein, 7 aa, Artificial Sequence
  peptide
VNPADYG

SEQ ID NO: 152 - protein, 11 aa, Artificial Sequence
  peptide
GGGSLRRNPAD

SEQ ID NO: 153 - protein, 9 aa, Artificial Sequence
  peptide
RPRWNPADY

SEQ ID NO: 154 - protein, 11 aa, Artificial Sequence
  peptide
GSLRWNPADYP

SEQ ID NO: 155 - protein, 9 aa, Artificial Sequence
  peptide
SLRYNPADY

SEQ ID NO: 156 - protein, 7 aa, Artificial Sequence
  peptide
RANPADY

SEQ ID NO: 157 - protein, 11 aa, Artificial Sequence
  peptide
GSLRINPADYG

SEQ ID NO: 158 - protein, 9 aa, Artificial Sequence
  peptide
RPNPADYGG

SEQ ID NO: 159 - protein, 7 aa, Artificial Sequence
  peptide
NPADYGY

SEQ ID NO: 160 - protein, 9 aa, Artificial Sequence
  peptide
ALRWNPADY

SEQ ID NO: 161 - protein, 11 aa, Artificial Sequence
  peptide
GGSLRYNPADY

SEQ ID NO: 162 - protein, 9 aa, Artificial Sequence
  peptide
RIRWNPADY

SEQ ID NO: 163 - protein, 7 aa, Artificial Sequence
  peptide
RWNPADR

SEQ ID NO: 164 - protein, 9 aa, Artificial Sequence
  peptide
RLHWNPADY

SEQ ID NO: 165 - protein, 9 aa, Artificial Sequence
  peptide
QLRWNPADY

SEQ ID NO: 166 - protein, 9 aa, Artificial Sequence
  peptide
RLRQNPADY

SEQ ID NO: 167 - protein, 11 aa, Artificial Sequence
  peptide
DVRLRWNPSDY

SEQ ID NO: 168 - protein, 11 aa, Artificial Sequence
  peptide
VRLRWNPADYS

SEQ ID NO: 169 - protein, 11 aa, Artificial Sequence
  peptide
DVRLRYNPADY

SEQ ID NO: 170 - protein, 11 aa, Artificial Sequence
  peptide
GSLRFNPADYG

SEQ ID NO: 171 - protein, 11 aa, Artificial Sequence
  peptide
GGSLRWNPADR

SEQ ID NO: 172 - protein, 9 aa, Artificial Sequence
  peptide
LRWNPADYR

SEQ ID NO: 173 - protein, 11 aa, Artificial Sequence
  peptide
LRWNPADYGGF

SEQ ID NO: 174 - protein, 11 aa, Artificial Sequence
  peptide
GSLRWNPADYD

SEQ ID NO: 175 - protein, 11 aa, Artificial Sequence
  peptide
RLRWNPADYHG

SEQ ID NO: 176 - protein, 11 aa, Artificial Sequence
  peptide
RLRWNPADYGP

SEQ ID NO: 177 - protein, 11 aa, Artificial Sequence
  peptide
GSLRWNPADYN

SEQ ID NO: 178 - protein, 11 aa, Artificial Sequence
  peptide
VRLRINPADYG

SEQ ID NO: 179 - protein, 9 aa, Artificial Sequence
  peptide
LRPNPADYG

SEQ ID NO: 180 - protein, 9 aa, Artificial Sequence
  peptide
TLRWNPADY

SEQ ID NO: 181 - protein, 9 aa, Artificial Sequence
  peptide
VRLRGNPAD

SEQ ID NO: 182 - protein, 9 aa, Artificial Sequence
  peptide
RSRWNPADY

SEQ ID NO: 183 - protein, 9 aa, Artificial Sequence
  peptide
RLRPNPADY

SEQ ID NO: 184 - protein, 9 aa, Artificial Sequence
  peptide
RLLWNPADY

SEQ ID NO: 185 - protein, 11 aa, Artificial Sequence
  peptide
RLRWNPADYGS

SEQ ID NO: 186 - protein, 11 aa, Artificial Sequence
  peptide
GSLRWNPADYQ

SEQ ID NO: 187 - protein, 11 aa, Artificial Sequence
  peptide
VRLRLNPADYG

SEQ ID NO: 188 - protein, 9 aa, Artificial Sequence
  peptide
SLYWNPADY

SEQ ID NO: 189 - protein, 7 aa, Artificial Sequence
  peptide
LRGNPAD

SEQ ID NO: 190 - protein, 7 aa, Artificial Sequence
  peptide
RWNPADF

SEQ ID NO: 191 - protein, 11 aa, Artificial Sequence
  peptide
RLRGNPADYGG

SEQ ID NO: 192 - protein, 7 aa, Artificial Sequence
  peptide
WNPDDYR

SEQ ID NO: 193 - protein, 11 aa, Artificial Sequence
  peptide
LRWNPADYPGI

SEQ ID NO: 194 - protein, 11 aa, Artificial Sequence
  peptide
DVRLRQNPADY

SEQ ID NO: 195 - protein, 11 aa, Artificial Sequence
  peptide
SLRWNPADYHG

SEQ ID NO: 196 - protein, 11 aa, Artificial Sequence
  peptide
GSLRWNPADYT

SEQ ID NO: 197 - protein, 7 aa, Artificial Sequence
  peptide
NPADYGN

SEQ ID NO: 198 - protein, 7 aa, Artificial Sequence
  peptide
LRFNPAD

SEQ ID NO: 199 - protein, 9 aa, Artificial Sequence
  peptide
RLRTNPADY

SEQ ID NO: 200 - protein, 11 aa, Artificial Sequence
  peptide
GGSLRLNPADY

SEQ ID NO: 201 - protein, 13 aa, Artificial Sequence
  peptide
WVDYNLKWNPDDY

SEQ ID NO: 202 - protein, 10 aa, Artificial Sequence
  peptide
YNLKWNPDDY

SEQ ID NO: 203 - protein, 13 aa, Artificial Sequence
  peptide
LFSHLQNEQWVDY

SEQ ID NO: 204 - protein, 7 aa, Artificial Sequence
  peptide
KWNPDDY

SEQ ID NO: 205 - protein, 7 aa, Artificial Sequence
  peptide
NEQWVDY

SEQ ID NO: 206 - protein, 10 aa, Artificial Sequence
  peptide
HLQNEQWVDY

SEQ ID NO: 207 - protein, 25 aa, Artificial Sequence
  peptide
CSGRVEVKVQEEWGTVCNNGWSMEA

SEQ ID NO: 208 - protein, 11 aa, Artificial Sequence
  peptide
GRVEVKVQEEW

SEQ ID NO: 209 - protein, 10 aa, Artificial Sequence
  peptide
WGTVCNNGWS

SEQ ID NO: 210 - protein, 9 aa, Artificial Sequence
  peptide
WGTVCNNGW

SEQ ID NO: 211 - protein, 13 aa, Artificial Sequence
  peptide
EWGTVCNNGWSME

SEQ ID NO: 212 - protein, 13 aa, Artificial Sequence
  peptide
QEEWGTVCNNGWS

SEQ ID NO: 213 - protein, 13 aa, Artificial Sequence
  peptide
WGTVCNNGWSMEA

SEQ ID NO: 214 - protein, 13 aa, Artificial Sequence
  peptide
EEWGTVCNNGWSM

SEQ ID NO: 215 - protein, 13 aa, Artificial Sequence
  peptide
VQEEWGTVCNNGW

SEQ ID NO: 216 - protein, 10 aa, Artificial Sequence
  peptide
EWGTVCNNGW

SEQ ID NO: 217 - protein, 10 aa, Artificial Sequence
  peptide
WGTVCNNGWS

SEQ ID NO: 218 - protein, 21 aa, Artificial Sequence
  peptide
DHVSCRGNESALWDCKHDGWG

SEQ ID NO: 219 - protein, 5 aa, Artificial Sequence
  peptide
ESALW

SEQ ID NO: 220 - protein, 7 aa, Artificial Sequence
  peptide
ESALWDC

SEQ ID NO: 221 - protein, 10 aa, Artificial Sequence
  peptide
RGNESALWDC

SEQ ID NO: 222 - protein, 10 aa, Artificial Sequence
  peptide
SCRGNESALW

SEQ ID NO: 223 - protein, 13 aa, Artificial Sequence
  peptide
VSCRGNESALWDC

SEQ ID NO: 224 - protein, 10 aa, Artificial Sequence
  peptide
ALWDCKHDGW

SEQ ID NO: 225 - protein, 13 aa, Artificial Sequence
  peptide
DHVSCRGNESALW

SEQ ID NO: 226 - protein, 10 aa, Artificial Sequence
  peptide
CRGNESALWD

SEQ ID NO: 227 - protein, 13 aa, Artificial Sequence
  peptide
NESALWDCKHDGW

SEQ ID NO: 228 - protein, 13 aa, Artificial Sequence
  peptide
ESALWDCKHDGWG

SEQ ID NO: 229 - protein, 20 aa, Artificial Sequence
  peptide
SSLGGTDKELRLVDGENKCS

SEQ ID NO: 230 - protein, 11 aa, Artificial Sequence
  peptide
SSLGGTDKELR

SEQ ID NO: 231 - protein, 5 aa, Artificial Sequence
  peptide
SSLGG

SEQ ID NO: 232 - protein, 10 aa, Artificial Sequence
  peptide
SSLGGTDKEL

SEQ ID NO: 233 - protein, 9 aa, Artificial Sequence
  peptide
SSLGGTDKE

SEQ ID NO: 234 - protein, 8 aa, Artificial Sequence
  peptide
SSLGGTDK

SEQ ID NO: 235 - protein, 7 aa, Artificial Sequence
  peptide
SSLGGTD

SEQ ID NO: 236 - protein, 6 aa, Artificial Sequence
  peptide
SSLGGT

SEQ ID NO: 237 - protein, 7 aa, Artificial Sequence
  peptide
LRRNPAD

SEQ ID NO: 238 - protein, 7 aa, Artificial Sequence
  peptide
NPADYRG

SEQ ID NO: 239 - protein, 7 aa, Artificial Sequence
  peptide
NPADYHG

SEQ ID NO: 240 - protein, 11 aa, Artificial Sequence
  peptide
VRLRWNPADYP

SEQ ID NO: 241 - protein, 7 aa, Artificial Sequence
  peptide
LRGNPAD

SEQ ID NO: 242 - protein, 7 aa, Artificial Sequence
  peptide
WNPADYR

SEQ ID NO: 243 - protein, 7 aa, Artificial Sequence
  peptide
LRFNPAD

SEQ ID NO: 244 - protein, 7 aa, Artificial Sequence
  peptide
GSLRYNP

SEQ ID NO: 245 - protein, 9 aa, Artificial Sequence
  peptide
LRVNPADYG

SEQ ID NO: 246 - protein, 9 aa, Artificial Sequence
  peptide
LRRNPADYG

SEQ ID NO: 247 - protein, 11 aa, Artificial Sequence
  peptide
VRLRWNPADYP

SEQ ID NO: 248 - protein, 9 aa, Artificial Sequence
  peptide
RLRVNPADY

SEQ ID NO: 249 - protein, 9 aa, Artificial Sequence
  peptide
LRVNPADYG

SEQ ID NO: 250 - protein, 11 aa, Artificial Sequence
  peptide
WIDVRLRGNPA

SEQ ID NO: 251 - protein, 7 aa, Artificial Sequence
  peptide
RLNPADY

SEQ ID NO: 252 - protein, 7 aa, Artificial Sequence
  peptide
RFNPADY

SEQ ID NO: 253 - protein, 9 aa, Artificial Sequence
  peptide
RLRLNPADY

SEQ ID NO: 254 - protein, 9 aa, Artificial Sequence
  peptide
RLRGNPADY

SEQ ID NO: 255 - protein, 11 aa, Artificial Sequence
  peptide
DVRLRINPADY

SEQ ID NO: 256 - protein, 11 aa, Artificial Sequence
  peptide
DVRLRVNPADY

SEQ ID NO: 257 - protein, 13 aa, Artificial Sequence
  peptide
LFSHLQNEQWVDY

SEQ ID NO: 258 - protein, 15 aa, Artificial Sequence
  peptide
CGRPQKRPSCIGCKG

SEQ ID NO: 259 - protein, 22 aa, Artificial Sequence
  peptide
LKWNPDDYGGVKKIHIPSEKGC

SEQ ID NO: 260 - protein, 13 aa, Artificial Sequence
  peptide
DHTLYTPYHTHPG

SEQ ID NO: 261 - protein, 11 aa, Artificial Sequence
  peptide
GRPQKRPSCIG

SEQ ID NO: 262 - protein, 11 aa, Artificial Sequence
  peptide
VKKIHIPSEKG

SEQ ID NO: 263 - protein, 12 aa, Artificial Sequence
  peptide
GGVKKIHIPSEK

SEQ ID NO: 264 - protein, 10 aa, Artificial Sequence
  peptide
IPNPLLGLDC

SEQ ID NO: 265 - protein, 23 aa, Artificial Sequence
  peptide
IPNPLLGLDGGSGDYKDDDDKGK

SEQ ID NO: 266 - protein, 24 aa, Artificial Sequence
  peptide
VKKIHIPSEKGGSGDYKDDDDKGK

SEQ ID NO: 267 - protein, 13 aa, Artificial Sequence
  peptide
CSGRVEVKVQEEW

SEQ ID NO: 268 - protein, 36 aa, Artificial Sequence
  peptide
DGENKCSGRVEVKVQEEWGTVCNNGWSMEAVSVICN

SEQ ID NO: 269 - protein, 33 aa, Artificial Sequence
  peptide
RIWMDHVSCRGNESALWDCKHDGWGKHSNCTHQ

SEQ ID NO: 270 - protein, 13 aa, Artificial Sequence
  peptide
VTNAPGEMKKELR

SEQ ID NO: 271 - protein, 13 aa, Artificial Sequence
  peptide
ASAVTNAPGEMKK

SEQ ID NO: 272 - protein, 10 aa, Artificial Sequence
  peptide
VTNAPGEMKK

SEQ ID NO: 273 - protein, 7 aa, Artificial Sequence
  peptide
VTNAPGE

SEQ ID NO: 274 - protein, 13 aa, Artificial Sequence
  peptide
GSASAVTNAPGEM

SEQ ID NO: 275 - protein, 13 aa, Artificial Sequence
  peptide
AVTNAPGEMKKEL

SEQ ID NO: 276 - protein, 13 aa, Artificial Sequence
  peptide
SASAVTNAPGEMK

SEQ ID NO: 277 - protein, 13 aa, Artificial Sequence
  peptide
SGSASAVTNAPGE

SEQ ID NO: 278 - protein, 10 aa, Artificial Sequence
  peptide
AVTNAPGEMK

SEQ ID NO: 279 - protein, 10 aa, Artificial Sequence
  peptide
SAVTNAPGEM

SEQ ID NO: 280 - protein, 10 aa, Artificial Sequence
  peptide
ASAVTNAPGE

SEQ ID NO: 281 - protein, 13 aa, Artificial Sequence
  peptide
SAVTNAPGEMKKE

SEQ ID NO: 282 - protein, 10 aa, Artificial Sequence
  peptide
TNAPGEMKKE

SEQ ID NO: 283 - protein, 22 aa, Artificial Sequence
  peptide
VTNAPGEMKKELRLAGGENNCS

SEQ ID NO: 284 - protein, 20 aa, Artificial Sequence
  peptide
SSLGGTDKELRLVDGENKCS

SEQ ID NO: 285 - protein, 29 aa, Artificial Sequence
  peptide
FSHLQNEQWVDYNLKWNPDDYGGVKKIHI

SEQ ID NO: 286 - protein, 29 aa, Artificial Sequence
  peptide
ETRLVANLLGGGSLRWNPADYGGIKKIRG

SEQ ID NO: 287 - protein, 116 aa, Artificial Sequence
  heavy chain variable (VH) region
DVQLQESGPGLVKPSQSLSLTCTVTGYSITSDYAWNWIRQFPGNKLEWMGYITYSGITNYNPSLKSQISITRDTSKNQFFLQLNSVTTEDTATYYCVSGTYYFDYWGQGTTLTVSS

SEQ ID NO: 288 - protein, 109 aa, Artificial Sequence
  light-chain variable (VL) region
SVVMTQTPKSLLISIGDRVTITCKASQSVSSDVAWFQQKPGQSPKPLIYYASNRYTGVPDRFTGSGYGTDFTFTISSVQAEDLAVYFCGQDYTSPRTFGGGTKLEIKRA

(19) United States
(12) Patent Application Publication
(10) Pub. No.: US 2023/0381328 A1
(43) Pub. Date: Nov. 30, 2023

(54) COMPOUND FOR THE PREVENTION OR TREATMENT OF MYASTHENIA GRAVIS

(71) Applicant: ABLEVIA BIOTECH GMBH, Wien (AT)

(72) Inventors: Oskar SMRZKA, Wien (AT); Bettina WANKO, Wien (AT)

(21) Appl. No.: 18/245,966

(22) PCT Filed: Sep. 23, 2021
(86) PCT No.: PCT/EP2021/076169

§ 371 Date: Mar. 20, 2023

(30) Foreign Application Priority Data
Sep. 24, 2020 (EP) 20198234.5

(51) Int. Cl. A61K 47/64; A61P 21/04; C07K 14/79; C07K 14/705; C07K 17/06

(52) U.S. Cl. CPC A61K 47/644; A61P 21/04; C07K 14/79; C07K 14/70571; C07K 17/06

(57) ABSTRACT

A compound for the sequestration of anti human muscle nicotinic acetylcholine receptor (AChR) autoantibodies, which are involved in the pathogenesis of MG is provided. The compound includes an inert biopolymer scaffold and at least two peptides with a sequence length of 6-13 amino acids, wherein each of the peptides independently includes an amino-acid sequence including an AChR main immunogenic region (MIR) epitope or mimotope. Also provided are pharmaceutical compositions including the compound, as well as a method of sequestering one or more antibodies present in an individual.

The field of present invention relates to the therapy of myasthenia gravis (MG).

MG is an autoimmune neuromuscular disorder mediated by autoantibodies that cause a broad spectrum of several clinical symptoms from mild muscle weakness to a life-threatening myasthenic crisis with breathing problems. Around 80% of myasthenic patients develop anti nicotinic acetylcholine receptor (AChR) antibodies that lead to complement-mediated damaging of the postsynaptic membrane (Howard 2018), direct AChR blocking or receptor endocytosis. These disease-causing autoantibodies are mainly directed to defined immunogenic regions AChR or MuSK (Ruff 2018). They represent a good example for functionally well characterized, disease-causing autoantibodies. Similar anti-AChR autoantibodies were also observed in rare neuroimmunological conditions (summarized as autoimmune channelopathies; Huang et al, 2019), such as autoimmune autonomic ganglionopathy (Nakane et al, 2018) or Morvan Syndrome (Masood 2021), or in paraneoplastic neurological syndromes (reviewed by Joubert et al, 2015). The main immunogenic region (MIR) is a conformation-dependent region at the extracellular apex of al subunits of muscle nicotinic AChR that is the target of half or more of the autoantibodies to muscle AChRs in human MG and rat experimental autoimmune MG (Luo et al., 2009).

Although general immunosuppressive or B-cell targeting strategies exist against MG, a strategy is needed whereby only diseases-causing antibodies (rather than all, mostly protective antibodies) are rapidly inactivated or depleted (especially in a myasthenic crisis), since neither of the generally immunosuppressive treatments with corticoids, IVIG, thymectomy or by plasma exchange are satisfactory. So far, no convenient therapeutic intervention exists that can deplete or neutralize disease causing antibodies in MG rapidly and selectively.

Rey et al. concerns the characterization of human antiacetylcholine receptor monoclonal autoantibodies from the peripheral blood of a MG patient using combinatorial libraries.

Trinh et al., 2014, relates to the design, synthesis, and characterization of a 39 amino acid peptide mimic of the MIR of the Torpedo AChR.

Yoshikawa, et al., 1997, concerns preventing the induction of rat experimental autoimmune MG with the compound FK506. FK506 is a macrolide compound isolated from Streptomyces tsukubanesis. The compound is taught to have a potent immunosuppressive property brought about by selective inhibition of helper T cell activation

WO 2018/049053 A2 relates to peptides and uses thereof for diagnosing and treating MG. It is disclosed that peptides can be administered to a subject with MG to bind autoantibodies circulating in the subject to form neutralizing complexes. The neutralizing complexes can be removed by affinity plasmapheresis.

U.S. Pat. No. 5,578,496 A concerns the detection of autoantibodies, in particular associated with MG, and treatment of autoimmune diseases, in particular MG. More specifically, the document discloses a method for construction of modified peptides covalently attached to a polymer which is asserted to render the synthetic peptide tolerogenic (“tolerogenic polymer”) and the use of these modified synthetic peptides in the treatment of diseases of autoimmunity and other unwanted responses such as allergic reactions and graft rejections. The tolerogenic polymer is disclosed to be e.g. monomethoxypoly-ethylene glycol (mPEG), polyvinyl alcohol (PVA) or polysaccharides such as dextrans.

EP 2 698 386 A1 relates to a fusion protein which is asserted to specifically suppress autoantibodies such as autoantibodies involved in MG. The fusion protein contains a binding site for the autoantibody and a fragment of the antibody heavy chain constant region which exhibits antibody-dependent cellular cytotoxicity.

Non-selective B-cell targeting or immunotherapeutic approaches are not yet an established therapeutic option for the treatment of MG. Alternatively, few intra- and extracorporeal selective antibody depletion or B-cell suppression strategies targeting disease-causing antibodies in MG were proposed using indirect or direct targeting approaches against disease causing antibodies (see e.g. Homma 2017 and Lazaridis 2017). In addition, an AChR-specific immunosuppressive therapy using an adjuvanted AChR vaccine was proposed (Luo 2015). However, there remains an urgent need for a comparatively effective and safe and rapidly acting selective antibody depletion therapy.

It is thus an object of the present invention to provide compounds and methods for selective depletion of MG-associated autoantibodies, to be used in therapy of MG. Preferably, these compounds and methods are more effective, safer (i.e. with less side effects) and/or more rapidly acting when compared to known MG therapies.

The present invention provides a compound (typically for the sequestration, or depletion, of antibodies, in particular anti nicotinic AChR antibodies, present in an individual) comprising
a biopolymer scaffold and at least
a first peptide n-mer of the general formula:

P(—S—P)₍ₙ₋₁₎ and
a second peptide n-mer of the general formula:

P(—S—P)₍ₙ₋₁₎.

Independently for each occurrence, P is a peptide with a sequence length of 6-13 amino acids, and S is a non-peptide spacer. Independently for each of the peptide n-mers, n is an integer of at least 1, preferably of at least 2, more preferably of at least 3, especially of at least 4. Each of the peptide n-mers is bound to the biopolymer scaffold, preferably via a linker each. Further, independently for each occurrence, P comprises at least 6, preferably at least 7, consecutive amino acids of a (preferably human nicotinic AChR MIR-derived) amino-acid sequence selected from SEQ ID NOs: 1-100 (as listed in Table 1 below), SEQ ID NOs: 101-200 (as listed in Table 2 below) and SEQ ID NOs: 201-206 (as listed in Table 3 below). Optionally, at most two, preferably at most one amino acid of said amino-acid sequence is independently substituted by any other amino acid.

Preferably, at least one occurrence of P is Pₐ and/or at least one occurrence of P is P_{b}. Pₐ is a defined peptide (i.e. a peptide of defined sequence) with a sequence length of 6-13 amino acids, preferably 7-13 amino acids. P_{b} is a defined peptide (i.e. a peptide of defined sequence) with a sequence length of 6-13 amino acids, preferably 7-13 amino acids.

The present invention also provides a compound comprising
a biopolymer scaffold and at least
a first peptide n-mer which is a peptide dimer of the formula Pₐ—S—Pₐ or Pₐ—S—P_{b}, wherein Pₐ is a defined peptide (i.e. a peptide of defined sequence) with a sequence length of 6-13 amino acids, preferably 7-11 amino acids, more preferably 7-9 amino acids, P_{b} is a defined peptide (i.e. a peptide of defined sequence) with a sequence length of 6-13 amino acids, preferably 7-13 amino acids, and S is a non-peptide spacer, wherein the first peptide n-mer is bound to the biopolymer scaffold, preferably via a linker. Pₐ comprises at least 6, preferably at least 7, consecutive amino acids of a (preferably human nicotinic AChR MIR-derived) amino-acid sequence selected from SEQ ID NOs: 1-100, SEQ ID NOs: 101-200 and SEQ ID NOs: 201-206.

This compound preferably comprises a second peptide n-mer which is a peptide dimer of the formula P_{b}—S—P_{b} or Pₐ—S—P_{b}, wherein the second peptide n-mer is bound to the biopolymer scaffold, preferably via a linker. P_{b} comprises at least 6, preferably at least 7, consecutive amino acids of a (preferably human nicotinic AChR MIR-derived) amino-acid sequence selected from SEQ ID NOs: 1-100, SEQ ID NOs: 101-200 and SEQ ID NOs: 201-206.

Furthermore, the present invention provides a pharmaceutical composition comprising any one of the aforementioned compounds and at least one pharmaceutically acceptable excipient.

Preferably, this pharmaceutical composition is for use in therapy, in particular for use in prevention or treatment of MG, especially transient neonatal MG, or of an autoimmune channelopathy such as autoimmune autonomic ganglionopathy or Morvan syndrome, or of a paraneoplastic neurological syndrome in an individual.

In another aspect, the present invention provides a method of sequestering (or depleting) one or more antibodies present in an individual, comprising obtaining a pharmaceutical composition as defined herein, the composition being non-immunogenic in the individual, where the one or more antibodies present in the individual are specific for at least one occurrence of P, or for peptide Pₐ and/or peptide P_{b}; and administering the pharmaceutical composition to the individual.

In even yet another aspect, the present invention provides a method of ameliorating or treating MG (or an autoimmune channelopathy such as autoimmune autonomic ganglionopathy or Morvan syndrome, or a paraneoplastic neurological syndrome) in an individual in need thereof, comprising obtaining a pharmaceutical composition as defined herein; and administering an effective amount of the pharmaceutical composition to the individual.

In the course of the present invention, a compound was developed which is able to deplete (or sequester) MG-associated autoantibodies in vivo and is therefore suitable for therapy of MG.

Further, it was surprisingly found that the approach which is also used in the invention is particularly effective in reducing titres of undesired antibodies in an individual. In particular, the compound achieved especially good results with regard to selectivity, duration of titre reduction and/or level of titre reduction in an in vivo model (see experimental examples). Furthermore, in the course of the present invention, linear and cyclic mimotopes and epitopes which bind particularly efficiently to known MG autoantibodies were found (see in particular Examples 14 and 15). These are especially suitable to be used in the compound of the present invention.

The detailed description given below relates to all of the above aspects of the invention unless explicitly excluded.

In general, antibodies are essential components of the humoral immune system, offering protection from infections by foreign organisms including bacteria, viruses, fungi or parasites. However, under certain circumstances—including autoimmune diseases, organ transplantation, blood transfusion or upon administration of biomolecular drugs or gene delivery vectors—antibodies can target the patient's own body (or the foreign tissue or cells or the biomolecular drug or vector just administered), thereby turning into harmful or disease-causing entities. Certain antibodies can also interfere with probes for diagnostic imaging. In the following, such antibodies are generally referred to as “undesired antibodies” or “undesirable antibodies”.

With few exceptions, selective removal of undesired antibodies has not reached clinical practice. It is presently restricted to very few indications: One of the known techniques for selective antibody removal (although not widely established) is immunoapheresis. In contrast to immunoapheresis (which removes immunoglobulin), selective immunoapheresis involves the filtration of plasma through an extracorporeal, selective antibody-adsorber cartridge that will deplete the undesired antibody based on selective binding to its antigen binding site. Selective immunoapheresis has for instance been used for removing anti-A or anti-B antibodies from the blood prior to ABO-incompatible transplantation or with respect to indications in transfusion medicine (Teschner et al). Selective apheresis was also experimentally applied in other indications, such as neuroimmunological indications (Tetala et al) or MG (Lazaridis et al), but is not yet established in the clinical routine. One reason that selective immunoapheresis is only hesitantly applied is the fact that it is a cost intensive and cumbersome intervention procedure that requires specialized medical care. Moreover, it is not known in the prior art how to deplete undesired antibodies rapidly and efficiently.

Unrelated to apheresis, Morimoto et al. discloses dextran as a generally applicable multivalent scaffold for improving immunoglobulin-binding affinities of peptide and peptidomimetic ligands such as the FLAG peptide. WO 2011/130324 A1 relates to compounds for prevention of cell injury. EP 3 059 244 A1 relates to a C-met protein agonist.

As mentioned, apheresis is applied extracorporeally. By contrast, also several approaches to deplete undesirable antibodies intracorporeally were proposed in the prior art, mostly in connection with certain autoimmune diseases involving autoantibodies or anti-drug antibodies:

Lorentz et al discloses a technique whereby erythrocytes are charged in situ with a tolerogenic payload driving the deletion of antigen-specific T cells. This is supposed to ultimately lead to reduction of the undesired humoral response against a model antigen. A similar approach is proposed in Pishesha et al. In this approach, erythrocytes are loaded ex vivo with a peptide-antigen construct that is covalently bound to the surface and reinjected into the animal model for general immunotolerance induction.

WO 92/13558 A1 relates to conjugates of stable nonimmunogenic polymers and analogs of immunogens that possess the specific B cell binding ability of the immunogen and which, when introduced into individuals, induce humoral anergy to the immunogen. Accordingly, these conjugates are disclosed to be useful for treating antibody-mediated pathologies that are caused by foreign- or self-immunogens. In this connection, see auch EP 0 498 658 A2.

Taddeo et al discloses selectively depleting antibody producing plasma cells using anti-CD138 antibody derivatives fused to an ovalbumin model antigen thereby inducing receptor crosslinking and cell suicide in vitro selectively in those cells that express the antibody against the model antigen.

Apitope International NV (Belgium) is presently developing soluble tolerogenic T-cell epitope peptides which may lead to expression of low levels of co-stimulatory molecules from antigen presenting cells inducing tolerance, thereby suppressing antibody response (see e.g. Jansson et al). These products are currently under preclinical and early clinical evaluation, e.g. in multiple sclerosis, Grave's disease, intermediate uveitis, and other autoimmune conditions as well as Factor VIII intolerance.

Similarly, Selecta Biosciences, Inc. (USA) is currently pursuing strategies of tolerance induction by so-called Synthetic Vaccine Particles (SVPs). SVP-Rapamycin is supposed to induce tolerance by preventing undesired antibody production via selectively inducing regulatory T cells (see Mazor et al).

Mingozzi et al discloses decoy adeno-associated virus (AAV) capsids that adsorb antibodies but cannot enter a target cell.

WO 2015/136027 A1 discloses carbohydrate ligands presenting the minimal Human Natural Killer-1 (HNK-1) epitope that bind to anti-MAG (myelin-associated glycoprotein) IgM antibodies, and their use in diagnosis as well as for the treatment of anti-MAG neuropathy. WO 2017/046172 A1 discloses further carbohydrate ligands and moieties, respectively, mimicking glycoepitopes comprised by glycosphingolipids of the nervous system which are bound by anti-glycan antibodies associated with neurological diseases. The document further relates to the use of these carbohydrate ligands/moieties in diagnosis as well as for the treatment of neurological diseases associated with anti-glycan antibodies.

US 2004/0258683 A1 discloses methods for treating systemic lupus erythematosus (SLE) including renal SLE and methods of reducing risk of renal flare in individuals with SLE, and methods of monitoring such treatment. One disclosed method of treating SLE including renal SLE and reducing risk of renal flare in an individual with SLE involves the administration of an effective amount of an agent for reducing the level of anti-double-stranded DNA (dsDNA) antibody, such as a dsDNA epitope as in the form of an epitope-presenting carrier or an epitope-presenting valency platform molecule, to the individual.

U.S. Pat. No. 5,637,454 relates to assays and treatments of autoimmune diseases. Agents used for treatment might include peptides homologous to the identified antigenic, molecular mimicry sequences. It is disclosed that these peptides could be delivered to a patient in order to decrease the amount of circulating antibody with a particular specificity.

US 2007/0026396 A1 relates to peptides directed against antibodies, which cause cold-intolerance, and the use thereof. It is taught that by using the disclosed peptides, in vivo or ex vivo neutralization of undesired autoantibodies is possible. A comparable approach is disclosed in WO 1992/014150 A1 or in WO 1998/030586 A2.

WO 2018/102668 A1 discloses a fusion protein for selective degradation of disease-causing or otherwise undesired antibodies. The fusion protein (termed “Seldeg”) includes a targeting component that specifically binds to a cell surface receptor or other cell surface molecule at near-neutral pH, and an antigen component fused directly or indirectly to the targeting component. Also disclosed is a method of depleting a target antigen-specific antibody from a patient by administering to the patient a Seldeg having an antigen component configured to specifically bind the target antigen-specific antibody.

WO 2015/181393 A1 concerns peptides grafted into sunflower-trypsin-inhibitor- (SFTI-) and cyclotide-based scaffolds. These peptides are disclosed to be effective in autoimmune disease, for instance citrullinated fibrinogen sequences that are grafted into the SFTI scaffold have been shown to block autoantibodies in rheumatoid arthritis and inhibit inflammation and pain. These scaffolds are disclosed to be non-immunogenic.

Erlandsson et al discloses in vivo clearing of idiotypic antibodies with anti-idiotypic antibodies and their derivatives.

Berlin Cures Holding AG (Germany) has proposed an intravenous broad spectrum neutralizer DNA aptamer (see e.g. WO 2016/020377 A1 and WO 2012/000889 A1) for the treatment of dilated cardiomyopathy and other GPCR-autoantibody related diseases that in high dosage is supposed to block autoantibodies by competitive binding to the antigen binding regions of autoantibodies. In general, aptamers did not yet achieve a breakthrough and are still in a preliminary stage of clinical development. The major concerns are still biostability and bioavailability, constraints such as nuclease sensitivity, toxicity, small size and renal clearance. A particular problem with respect to their use as selective antibody antagonists are their propensity to stimulate the innate immune response.

WO 00/33887 A2 discloses methods for reducing circulating levels of antibodies, particularly disease-associated antibodies. The methods entail administering effective amounts of epitope-presenting carriers to an individual. In addition, ex vivo methods for reducing circulating levels of antibodies are disclosed which employ epitope-presenting carriers.

U.S. Pat. No. 6,022,544 A relates to a method for reducing an undesired antibody response in a mammal by administering to the mammal a non-immunogenic construct which is free of high molecular weight immunostimulatory molecules. The construct is disclosed to contain at least two copies of a B cell membrane immunoglobulin receptor epitope bound to a pharmaceutically acceptable non-immunogenic carrier.

However, the approaches to deplete undesirable antibodies intracorporeally disclosed in the prior art have many shortcomings. In particular, neither of them has been approved for regular clinical use.

The biopolymer scaffold used in the present invention may be a mammalian biopolymer such as a human biopolymer, a non-human primate biopolymer, a sheep biopolymer, a pig biopolymer, a dog biopolymer or a rodent biopolymer. In particular the biopolymer scaffold is a protein, especially a (non-modified or non-modified with respect to its amino-acid sequence) plasma protein. Preferably, the biopolymer scaffold is a mammalian protein such as a human protein, a non-human primate protein, a sheep protein, a pig protein, a dog protein or a rodent protein.

Typically, the biopolymer scaffold is a non-immunogenic and/or non-toxic protein that preferably circulates in the plasma of healthy (human) individuals and can e.g. be efficiently scavenged or recycled by scavenging receptors, such as e.g. present on myeloid cells or on liver sinusoidal endothelial cells (reviewed by Sorensen et al 2015).

According to a particular preference, the biopolymer scaffold is a (preferably human) globulin, preferably selected from the group consisting of immunoglobulins, alpha1-globulins, alpha2-globulins and beta-globulins, in particular immunoglobulin G, haptoglobin and transferrin. Haptoglobin in particular has several advantageous properties, as shown in Examples 5-9, especially an advantageous safety profile.

The biopolymer scaffold may also be (preferably human) albumin, hemopexin, alpha-1-antitrypsin, C1 esterase inhibitor, lactoferrin or non-immunogenic (i.e. non-immunogenic in the individual to be treated) fragments of all of the aforementioned proteins, including the globulins.

In another preference, the biopolymer scaffold is an anti-CD163 antibody (i.e. an antibody specific for a CD163 protein) or CD163-binding fragment thereof.

Human CD163 (Cluster of Differentiation 163) is a 130 kDa membrane glycoprotein (formerly called M130) and prototypic class I scavenger receptor with an extracellular portion consisting of nine scavenger receptor cysteine-rich (SRCR) domains that are responsible for ligand binding. CD163 is an endocytic receptor present on macrophages and monocytes, it removes hemoglobin/haptoglobin complexes from the blood but it also plays a role in anti-inflammatory processes and wound healing. Highest expression levels of CD163 are found on tissue macrophages (e.g. Kupffer cells in the liver) and on certain macrophages in spleen and bone marrow. Because of its tissue- and cell-specific expression and entirely unrelated to depletion of undesirable antibodies, CD163 is regarded as a macrophage target for drug delivery of e.g. immunotoxins, liposomes or other therapeutic compound classes (Skytthe et al., 2020).

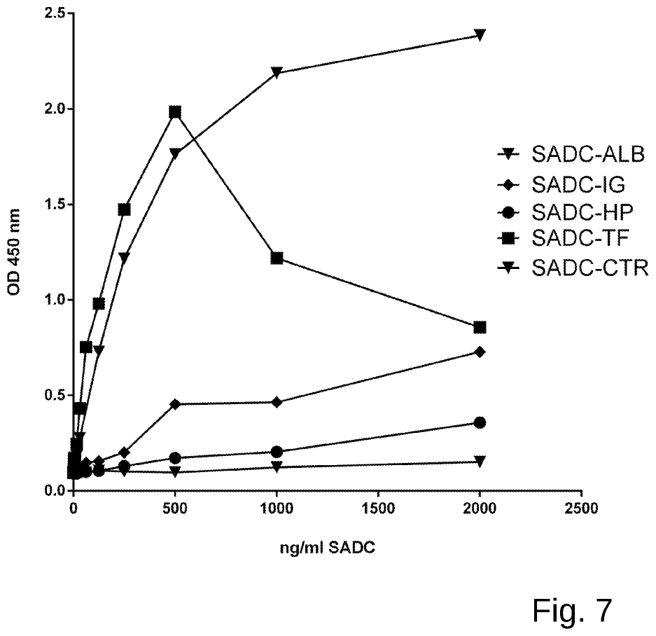

Monoclonal anti-CD163 antibodies and the SRCR domains they are binding are for instance disclosed in Madsen et al., 2004, in particular FIG. 7. Further anti-CD163 antibodies and fragments thereof are e.g. disclosed in WO 2002/032941 A2 or WO 2011/039510 A2. At least two structurally different binding sites for ligands were mapped by using domain-specific antibodies such as e.g. monoclonal antibody (mAb) EDhul (see Madsen et al, 2004). This antibody binds to the third SRCR of CD163 and competes with hemoglobin/haptoglobin binding to CD163. Numerous other antibodies against different domains of CD163 were previously described in the literature, including Mac2-158, KiM8, GHI/61 and RM3/1, targeting SRCR domains 1, 3, 7 and 9, respectively. In addition, conserved bacterial binding sites were mapped and it was demonstrated that certain antibodies were able to inhibit either bacterial binding but not hemoglobin/haptoglobin complex binding and vice versa. This points to different modes of binding and ligand interactions of CD163 (Fabriek et al, 2009; see also citations therein).

Entirely unrelated to depletion of undesirable antibodies, CD163 was proposed as a target for cell-specific drug delivery because of its physiological properties. Tumor-associated macrophages represent one of the main targets where the potential benefit of CD163-targeting is currently explored. Remarkably, numerous tumors and malignancies were shown to correlate with CD163 expression levels, supporting the use of this target for tumor therapy. Other proposed applications include CD163 targeting by anti-drug conjugates (ADCs) in chronic inflammation and neuroinflammation (reviewed in Skytthe et al., 2020). Therefore, CD163-targeting by ADCs notably with dexamethasone or stealth liposome conjugates represents therapeutic principle which is currently studied (Graversen et al., 2012; Etzerodt et al., 2012).

In that context, there are references indicating that anti-CD163 antibodies can be rapidly internalized by endocytosis when applied in vivo. This was shown for example for monoclonal antibody (mAb) Ed-2 (Dijkstra et al., 1985; Graversen et al., 2012) or for mAb Mac2-158/KN2/NRY (Granfeldt et al., 2013). Based on those observations in combination with observations made in the course of the present invention (see in particular example section), anti-CD163 antibodies and CD163-binding turned out to be highly suitable biopolymer scaffolds for depletion/sequestration of undesirable antibodies.

Numerous anti-CD163 antibodies and CD163-binding fragments thereof are known in the art (see e.g. above). These are suitable to be used as a biopolymer scaffold for the present invention. For instance, any anti-CD163 antibody or fragment thereof mentioned herein or in WO 2011/039510 A2 (which is included herein by reference) may be used as a biopolymer scaffold in the invention. Preferably, the biopolymer scaffold of the inventive compound is antibody Mac2-48, Mac2-158, 5C6-FAT, BerMac3, or E10B10 as disclosed in WO 2011/039510, in particular humanised Mac2-48 or Mac2-158 as disclosed in WO 2011/039510 A2.

In a preferred embodiment, the anti-CD163 antibody or CD163-binding fragment thereof comprises a heavy-chain variable (V_{H}) region comprising one or more complementarity-determining region (CDR) sequences selected from the group consisting of SEQ ID NOs: 11-13 of WO 2011/039510 A2.

In addition, or alternatively thereto, in a preferred embodiment, the anti-CD163 antibody or CD163-binding fragment thereof comprises a light-chain variable (V_{L}) region comprising one or more CDR sequences selected from the group consisting of SEQ ID NOs: 14-16 of WO 2011/039510 A2 or selected from the group consisting of SEQ ID NOs:17-19 of WO 2011/039510 A2.

In a further preferred embodiment, the anti-CD163 antibody or CD163-binding fragment thereof comprises a heavy-chain variable (V_{H}) region comprising or consisting of the amino acid sequence of SEQ ID NO: 20 of WO 2011/039510 A2.

In addition, or alternatively thereto, in a preferred embodiment, the anti-CD163 antibody or CD163-binding fragment thereof comprises a light-chain variable (V_{L}) region comprising or consisting of the amino acid sequence of SEQ ID NO: 21 of WO 2011/039510 A2.

In a further preferred embodiment, the anti-CD163 antibody or CD163-binding fragment thereof comprises a heavy-chain variable (V_{H}) region comprising or consisting of the amino acid sequence of SEQ ID NO: 22 of WO 2011/039510 A2.

In addition, or alternatively thereto, in a preferred embodiment, the anti-CD163 antibody or CD163-binding fragment thereof comprises a light-chain variable (V_{L}) region comprising or consisting of the amino acid sequence of SEQ ID NO: 23 of WO 2011/039510 A2.

In a further preferred embodiment, the anti-CD163 antibody or CD163-binding fragment thereof comprises a heavy-chain variable (V_{H}) region comprising or consisting of the amino acid sequence of SEQ ID NO: 24 of WO 2011/039510 A2.

In addition, or alternatively thereto, in a preferred embodiment, the anti-CD163 antibody or CD163-binding fragment thereof comprises a light-chain variable (V_{L}) region comprising or consisting of the amino acid sequence of SEQ ID NO: 25 of WO 2011/039510 A2.

In the context of the present invention, the anti-CD163 antibody may be a mammalian antibody such as a humanized or human antibody, a non-human primate antibody, a sheep antibody, a pig antibody, a dog antibody or a rodent antibody. In embodiments, the anti-CD163 antibody may monoclonal.

According to a preference, the anti-CD163 antibody is selected from IgG, IgA, IgD, IgE and IgM.

According to a further preference, the CD163-binding fragment is selected from a Fab, a Fab′, a F(ab)2, a Fv, a single-chain antibody, a nanobody and an antigen-binding domain.

CD163 amino acid sequences are for instance disclosed in WO 2011/039510 A2 (which is included here by reference). In the context of the present invention, the anti-CD163 antibody or CD163-binding fragment thereof is preferably specific for a human CD163, especially with the amino acid sequence of any one of SEQ ID NOs: 28-31 of WO 2011/039510 A2.

In a further preferred embodiment, the anti-CD163 antibody or CD163-binding fragment thereof is specific for the extracellular region of CD163 (e.g. for human CD163: amino acids 42-1050 of UniProt Q86VB7, sequence version 2), preferably for an SRCR domain of CD163, more preferably for any one of SRCR domains 1-9 of CD163 (e.g. for human CD163: amino acids 51-152, 159-259, 266-366, 373-473, 478-578, 583-683, 719-819, 824-926 and 929-1029, respectively, of UniProt Q86VB7, sequence version 2), even more preferably for any one of SRCR domains 1-3 of CD163 (e.g. for human CD163: amino acids 51-152, 159-259, 266-366, and 373-473, respectively, of UniProt Q86VB7, sequence version 2), especially for SRCR domain 1 of CD163 (in particular with the amino acid sequence of any one of SEQ ID NOs: 1-8 of WO 2011/039510 A2, especially SEQ ID NO: 1 of WO 2011/039510 A2).

In a particular preference, the anti-CD163 antibody or CD163-binding fragment thereof is capable of competing for binding to (preferably human) CD163 with a (preferably human) hemoglobin-haptoglobin complex (e.g. in an ELISA).

In another particular preference, the anti-CD163 antibody or CD163-binding fragment thereof is capable of competing for binding to human CD163 with any of the anti-human CD163 mAbs disclosed herein, in particular Mac2-48 or Mac2-158 as disclosed in WO 2011/039510 A2.

In yet another particular preference, the anti-CD163 antibody or CD163-binding fragment thereof is capable of competing for binding to human CD163 with an antibody having a heavy chain variable (VH) region consisting of the amino acid sequence

DVQLQESGPGLVKPSQSLSLTCTVTGYSITSDYAWNWIRQFPGNKLEWMG
YITYSGITNYNPSLKSQISITRDTSKNQFFLQLNSVTTEDTATYYCVSGT
YYFDYWGQGTTLTVSS,

and having a light-chain variable (VL) region consisting of the amino acid sequence

SVVMTQTPKSLLISIGDRVTITCKASQSVSSDVAWFQQKPGQSPKPLIYY
ASNRYTGVPDRFTGSGYGTDFTFTISSVQAEDLAVYFCGQDYTSPRTFGG
GTKLEIKRA

### (e.g. in an ELISA).

Details on competitive binding experiments are known to the person of skilled in the art (e.g. based on ELISA) and are for instance disclosed in WO 2011/039510 A2 (which is included herein by reference).

The epitopes of antibodies E10B10 and Mac2-158 as disclosed in WO 2011/039510 were mapped (see example section). These epitopes are particularly suitable for binding of the anti-CD163 antibody (or CD163-binding fragment thereof) of the inventive compound.

Accordingly, in particularly preferred embodiment, the anti-CD163 antibody or CD163-binding, fragment thereof is specific for peptide consisting of 7-25, preferably 8-20, even more preferably 9-15, especially 10-13 amino acids, wherein the peptide comprises the amino acid sequence CSGRVEVKVQEEWGTVCINNGWSMEA or a 7-24 amino-acid fragment thereof. Preferably, this peptide comprises the amino acid sequence GRVEVKVQEEW, WGTVCNNGWS or WGTVCNNGW. More preferably, the peptide comprises an amino acid sequence selected from EWGTVCNNGWSME, QEEWGTVCNNGWS, WGTVCNNGWSMEA, EEWGTVCNNGWSM, VQEEWGTVCNNGW, EWGTVCNNGW and WGTVCNNGWS. Even more preferably, the peptide consists of an amino acid sequence selected from EWGTVCNNGWSME, QEEWGTVCNNGWS, WGTVCNNGWSMEA, EEWGTVCNNGWSM, VQEEWGTVCNNGW, EWGTVCNNGW and WGTVCNNGWS, optionally with an N-terminal and/or C-terminal cysteine residue.

Accordingly, in another particularly preferred embodiment, the anti-CD163 antibody or CD163-binding fragment thereof is specific for a peptide consisting of 7-25, preferably 8-20, even more preferably 9-15, especially 10-13 amino acids, wherein the peptide comprises the amino acid sequence DHVSCRGNESALWDCKHDGWG or a 7-20 amino-acid fragment thereof. Preferably, this peptide comprises the amino acid sequence ESALW or ALW. More preferably, the peptide comprises an amino acid sequence selected from ESALWDC, RGNESALWDC, SCRGNESALW, VSCRGNESALWDC, ALWDCKHDGW, DlHVSCRGNESALW, CRGNESALWID, NESALVJDCKIDGW and ESALDCKHDGWG. Even more preferably, the peptide consists of an amino acid sequence selected from ESALVDC, RGNESALWDC, SCRGNESALW, VSCRCNESALWDC, ALWDCKHDGW, DHVSCRGNESALW, CRGNESALWD, NESALWDCKHDGW and ESALWDCKEDGWG, optionally with an N-terminal and/or C-terminal cysteine residue.

Accordingly, in another particularly preferred embodiment, the anti-CD163 antibody or CD163-binding fragment thereof is specific for a peptide consisting of 7-25, preferably 8-20, even more preferably 9-15, especially 10-13 amino acids, wherein the peptide comprises the amino acid sequence SSLGGTDKELRLVDGENKCS or a 7-19 amino-acid fragment thereof. Preferably, this peptide comprises the amino acid sequence SSLGGTDKELR or SSLXGG. More preferably, the peptide comprises an amino acid sequence selected from SSLGGTDKELR, SSLGGTDKEL, SSLGGTDKE, SSLGGTDK, SSLGGTD, SSLGGT and SSLGG. Even more preferably, the peptide consists of an amino acid sequence selected from SSLGGTDKELR, SSLGGTDKEL, SSLGCTDKE, SSLGCTDK, SSLGCTD, SSLGGT and SSLGG, optionally with an N-terminal and/or C-terminal cysteine residue.

The peptides (or peptide n-mers) are preferably covalently conjugated (or covalently bound) to the biopolymer scaffold via a (non-immunogenic) linker known in the art such as for example amine-to-sulfhydryl linkers and bifunctional NHS-PEG-maleimide linkers or other linkers known in the art. Alternatively, the peptides (or peptide n-mers) can be bound to the epitope carrier scaffold e.g. by formation of a disulfide bond between the protein and the peptide (which is also referred to as “linker” herein), or using non-covalent assembly techniques, spontaneous isopeptide bond formation or unnatural amino acids for bio-orthogonal chemistry via genetic code expansion techniques (reviewed by Howarth et al 2018 and Lim et al 2016).

The compound of the present invention may comprise e.g. at least two, preferably between 3 and 40 copies of one or several different peptides (which may be present in different forms of peptide n-mers as disclosed herein). The compound may comprise one type of epitopic peptide (in other words: antibody-binding peptide or paratope-binding peptide), however the diversity of epitopic peptides bound to one biopolymer scaffold molecule can be a mixture of e.g. up to 8 different epitopic peptides.

Typically, since the peptides present in the inventive compound specifically bind to selected undesired antibodies, their sequence is usually selected and optimized such that they provide specific binding in order to guarantee selectivity of undesired antibody depletion from the blood. For this purpose, the peptide sequence of the peptides typically corresponds to the entire epitope sequence or portions of the undesired antibody epitope. The peptides used in the present invention can be further optimized by exchanging one, two or up to four amino-acid positions, allowing e.g. for modulating the binding affinity to the undesired antibody that needs to be depleted. Such single or multiple amino-acid substitution strategies that can provide “mimotopes” with increased binding affinity and are known in the field and were previously developed using phage display strategies or peptide microarrays. In other words, the peptides used in the present invention do not have to be completely identical to the native epitope sequences of the undesired antibodies.

Typically, the peptides used in the compound of the present invention (e.g. peptide P or Pₐ or P_{b}) are composed of one or more of the 20 amino acids commonly present in mammalian proteins. In addition, the amino acid repertoire used in the peptides may be expanded to post-translationally modified amino acids e.g. affecting antigenicity of proteins such as post translational modifications, in particular oxidative post translational modifications (see e.g. Ryan 2014) or modifications to the peptide backbone (see e.g. Müller 2018), or to non-natural amino acids (see e.g. Meister et al 2018). These modifications may also be used in the peptides e.g. to adapt the binding interaction and specificity between the peptide and the variable region of an undesired antibody. In particular, epitopes (and therefore the peptides used in the compound of the present invention) can also contain citrulline as for example in autoimmune diseases. Furthermore, by introducing modifications into the peptide sequence the propensity of binding to an HLA molecule may be reduced, the stability and the physicochemical characteristics may be improved or the affinity to the undesired antibody may be increased.

In many cases, the undesired antibody that is to be depleted is oligo- or polyclonal (e.g. autoantibodies, ADAs or alloantibodies are typically poly- or oligoclonal), implying that undesired (polyclonal) antibody epitope covers a larger epitopic region of a target molecule. To adapt to this situation, the compound of the present invention may comprise a mixture of two or several epitopic peptides (in other words: antibody-binding peptides or paratope-binding peptides), thereby allowing to adapt to the polyclonality or oligoclonality of an undesired antibody.

Such poly-epitopic compounds of the present invention can effectively deplete undesired antibodies and are more often effective than mono-epitopic compounds in case the epitope of the undesired antibody extends to larger amino acid sequence stretches.

It is advantageous if the peptides used for the inventive compound are designed such that they will be specifically recognized by the variable region of the undesired antibodies to be depleted. The sequences of peptides used in the present invention may e.g. be selected by applying fine epitope mapping techniques (i.e. epitope walks, peptide deletion mapping, amino acid substitution scanning using peptide arrays such as described in Carter et al 2004, and Hansen et al 2013) on the undesired antibodies.

In the course of the present invention, mimotope and epitope screens with MG-related autoantibodies were performed (see Examples 14 and 15). Peptide mimotopes and epitopes were found which are particularly suitable for the compound of the present invention. These mimotopes and epitopes are listed in Tables 1-3 below.

Table 1 lists linear mimotopes, ranked by binding affinity to the MG-related autoantibody (“relative signal”)

| SEQ ID NO | mimotope sequence | relative signal [%] |
|---|---|---|
| 1 | LRRNPAD | 100.0 |
| 2 | NPADYRG | 71.4 |
| 3 | NPADYHG | 59.3 |
| 4 | VRLRWNPADYP | 56.7 |
| 5 | LRGNPAD | 48.0 |
| 6 | WNPADYR | 40.6 |
| 7 | LRFNPAD | 37.1 |
| 8 | GSLRYNP | 34.4 |
| 9 | LRVNPADYG | 31.6 |
| 10 | LRRNPADYG | 28.3 |
| 11 | VRLRFNP | 27.1 |
| 12 | VRLRYNP | 26.3 |
| 13 | LRLNPAD | 25.2 |
| 14 | VRLRRNPAD | 25.0 |
| 15 | RFNPADY | 21.8 |
| 16 | GSLRFNP | 21.6 |
| 17 | NPDDYHG | 21.3 |
| 18 | NPADYGY | 21.2 |
| 19 | WNPADYP | 20.5 |
| 20 | RLHWNPADY | 20.2 |
| 21 | QWIDVRLRFNP | 20.1 |
| 22 | NPADYPG | 19.8 |
| 23 | NPADYGH | 18.1 |
| 24 | RLNPADY | 17.7 |
| 25 | LRLNPADYG | 17.3 |
| 26 | LLGGGSLRFNP | 16.7 |
| 27 | RYNPADY | 16.5 |
| 28 | LRYNPAD | 15.9 |
| 29 | LHWNPAD | 15.7 |
| 30 | LRWNPADYH | 15.3 |
| 31 | IDVRLRFNP | 15.0 |
| 32 | RLRVNPADY | 14.9 |
| 33 | NPADYGD | 14.1 |
| 34 | WNPADYGHI | 13.7 |
| 35 | YNLKFNP | 13.7 |
| 36 | SLHWNPADY | 13.6 |
| 37 | QWIDVRLRYNP | 13.4 |
| 38 | RGNPADYGG | 13.0 |
| 39 | GGGSLRFNP | 13.0 |
| 40 | LRINPADYG | 12.6 |
| 41 | RWNPADF | 12.6 |
| 42 | RLRWNPADYHG | 12.4 |
| 43 | IDVRLRYNP | 12.2 |
| 44 | RINPADY | 12.2 |
| 45 | NPADYGR | 12.2 |
| 46 | RLRFNPADY | 11.4 |
| 47 | WNPDDYR | 11.4 |
| 48 | CNLKFNP | 11.0 |
| 49 | SLRINPADY | 11.0 |
| 50 | RLYWNPADY | 11.0 |
| 51 | QWVDYNLKFNP | 10.9 |
| 52 | GGGSLRRNPAD | 10.6 |
| 53 | LRWNPADYGGF | 10.6 |
| 54 | SLRWNPADYHG | 10.6 |
| 55 | NPADYDG | 10.3 |
| 56 | DVRLRFNPADY | 10.0 |
| 57 | RRNPADY | 9.9 |
| 58 | RPNPADY | 9.5 |
| 59 | GSLRRNPAD | 9.5 |
| 60 | WNPADYH | 9.4 |
| 61 | RWNPADYGH | 9.4 |
| 62 | GSLRFNPAD | 9.3 |
| 63 | LRINPAD | 9.2 |
| 64 | WRLRWNPAD | 9.0 |
| 65 | GNPADYGGI | 8.9 |
| 66 | RHNPADY | 8.9 |
| 67 | WNPDDYGHV | 8.8 |
| 68 | RPNPADYGG | 8.6 |
| 69 | DVRLRINPADY | 8.5 |
| 70 | YNLKYNP | 8.3 |
| 71 | GGGSLRFNPAD | 8.1 |
| 72 | VRHRWNPAD | 8.1 |
| 73 | NPADYGI | 8.0 |
| 74 | IDVRLRFNPAD | 7.9 |
| 75 | NPDDYGH | 7.7 |
| 76 | VPLRWNPAD | 7.6 |
| 77 | YNL PWNP | 7.6 |
| 78 | VDYNLKFNP | 7.6 |
| 79 | HWNPADY | 7.4 |
| 80 | RTNPADY | 7.2 |
| 81 | LRWNPADYHGI | 7.2 |
| 82 | GGGSLRYNP | 7.1 |
| 83 | RLRGNPADY | 6.9 |
| 84 | RGNPADY | 6.9 |
| 85 | VDCNLKFNP | 6.8 |
| 86 | DVRLRWNPSDY | 6.8 |
| 87 | RIRWNPADY | 6.8 |
| 88 | HRLRWNPAD | 6.7 |
| 89 | SLRVNPADY | 6.7 |
| 90 | CNLKYNP | 6.7 |
| 91 | HSLRWNPAD | 6.6 |
| 92 | NPADYGN | 6.5 |
| 93 | LRWNPADYPGI | 6.5 |
| 94 | RLRYNPADY | 6.5 |
| 95 | SLRFNPADY | 6.5 |
| 96 | QWVDCNLKFNP | 6.4 |
| 97 | LRPNPADYG | 6.1 |
| 98 | VDCNLKYNP | 6.1 |
| 99 | ALRWNPADY | 6.0 |
| 100 | NPADYGV | 6.0 |

Table 2 lists cyclic mimotopes, ranked by binding affinity to the MG-related autoantibody (“relative signal”)

| SEQ ID NO | mimotope sequence | relative signal [%] |
|---|---|---|
| 101 | VRLRWNPADYP | 100.0 |
| 102 | RLRVNPADY | 61.3 |
| 103 | LRVNPADYG | 45.1 |
| 104 | WIDVRLRGNPA | 43.2 |
| 105 | RLNPADY | 42.3 |
| 106 | RFNPADY | 37.5 |
| 107 | RLRLNPADY | 37.3 |
| 108 | RLRGNPADY | 32.7 |
| 109 | DVRLRINPADY | 31.4 |
| 110 | DVRLRVNPADY | 30.2 |
| 111 | RINPADY | 28.9 |
| 112 | LRLNPADYG | 28.6 |
| 113 | LRRNPAD | 28.2 |
| 114 | SLRINPADY | 28.0 |
| 115 | DVRLRFNPADY | 27.7 |
| 116 | NPADYRG | 27.4 |
| 117 | SLRVNPADY | 26.5 |
| 118 | RLRINPADY | 25.5 |
| 119 | GGSLRINPADY | 25.4 |
| 120 | DVRLRGNPADY | 25.0 |
| 121 | WNPADYR | 24.9 |
| 122 | RLRYNPADY | 24.5 |
| 123 | RGNPADY | 23.8 |
| 124 | RRNPADY | 23.5 |
| 125 | LRRNPADYG | 23.4 |
| 126 | RINPADYGG | 23.1 |
| 127 | LRINPADYG | 22.8 |
| 128 | GGSLRVNPADY | 22.5 |
| 129 | RGNPADYGG | 22.2 |
| 130 | SLRLNPADY | 21.8 |
| 131 | DVRLRPNPADY | 21.3 |
| 132 | RLRFNPADY | 21.3 |
| 133 | WNPADYP | 21.1 |
| 134 | RYNPADY | 20.2 |
| 135 | GGSLRGNPADY | 19.6 |
| 136 | NPADYGR | 19.5 |
| 137 | RVNPADY | 18.7 |
| 138 | GSLRWNPADYR | 16.2 |
| 139 | NPADYGH | 15.8 |
| 140 | RPNPADY | 15.6 |
| 141 | SLRGNPADY | 15.4 |
| 142 | VRLRRNPAD | 14.7 |
| 143 | RTNPADY | 14.6 |
| 144 | GGSLRFNPADY | 14.5 |
| 145 | RLYWNPADY | 13.7 |
| 146 | GNPADYGGI | 13.7 |
| 147 | SLRFNPADY | 13.6 |
| 148 | VRLRVNPADYG | 13.4 |
| 149 | LRWNPADYP | 13.1 |
| 150 | NPADYGD | 12.6 |
| 151 | VNPADYG | 12.5 |
| 152 | GGGSLRRNPAD | 12.5 |
| 153 | RPRWNPADY | 12.4 |
| 154 | GSLRWNPADYP | 12.4 |
| 155 | SLRYNPADY | 12.2 |
| 156 | RANPADY | 12.2 |
| 157 | GSLRINPADYG | 12.1 |
| 158 | RPNPADYGG | 11.9 |
| 159 | NPADYGY | 11.8 |
| 160 | ALRWNPADY | 11.5 |
| 161 | GGSLRYNPADY | 11.5 |
| 162 | RIRWNPADY | 11.5 |
| 163 | RWNPADR | 11.0 |
| 164 | RLHWNPADY | 10.8 |
| 165 | QLRWNPADY | 10.7 |
| 166 | RLRONPADY | 10.6 |
| 167 | DVRLRWNPSDY | 10.5 |
| 168 | VRLRWNPADYS | 10.5 |
| 169 | DVRLRYNPADY | 10.5 |
| 170 | GSLRFNPADYG | 10.4 |
| 171 | GGSLRWNPADR | 10.3 |
| 172 | LRWNPADYR | 10.3 |
| 173 | LRWNPADYGGF | 10.3 |
| 174 | GSLRWNPADYD | 10.2 |
| 175 | RLRWNPADYHG | 10.1 |
| 176 | RLRWNPADYGP | 10.0 |
| 177 | GSLRWNPADYN | 10.0 |
| 178 | VRLRINPADYG | 10.0 |
| 179 | LRPNPADYG | 10.0 |
| 180 | TLRWNPADY | 9.9 |
| 181 | VRLRGNPAD | 9.9 |
| 182 | RSRWNPADY | 9.8 |
| 183 | RLRPNPADY | 9.8 |
| 184 | RLLWNPADY | 9.7 |
| 185 | RLRWNPADYGS | 9.7 |
| 186 | GSLRWNPADYQ | 9.7 |
| 187 | VRLRLNPADYG | 9.7 |
| 188 | SLYWNPADY | 9.4 |
| 189 | LRGNPAD | 9.1 |
| 190 | RWNPADF | 9.1 |
| 191 | RLRGNPADYGG | 9.0 |
| 192 | WNPDDYR | 9.0 |
| 193 | LRWNPADYPGI | 8.9 |
| 194 | DVRLRONPADY | 8.8 |
| 195 | SLRWNPADYHG | 8.7 |
| 196 | GSLRWNPADYT | 8.7 |
| 197 | NPADYGN | 8.7 |
| 198 | LRFNPAD | 8.6 |
| 199 | RLRTNPADY | 8.6 |
| 200 | GGSLRLNPADY | 8.5 |

Table 3 shows cyclic epitopes of an MG-related autoantibody

| SEQ ID NO | epitope sequence |
|---|---|
| 201 | WVDYNLKWNPDDY |
| 202 | YNLKWNPDDY |
| 203 | LFSHLQNEQWVDY |
| 204 | KWNPDDY |
| 205 | NEQWVDY |
| 206 | HLQNEQWVDY |

In a preferred embodiment, independently for each occurrence, P comprises at least 6, preferably at least 7, more preferably at least 8, yet more preferably at least 9, even more preferably at least 10, yet even more preferably at least 11 or even 12, especially all consecutive amino acids of an amino-acid sequence selected from SEQ ID NOs: 1-100, SEQ ID NOs: 101-200 and SEQ ID NOs: 201-206, optionally wherein at most two, preferably at most one amino acid of said amino-acid sequence is independently substituted by any other amino acid.

Preferably, said amino sequence is selected from LRRNPAD, NPADYRG, NPADYHG, VRLRWNPADYP, LRGNPAD, WNPADYR, LRFNPAD, GSLRYNP, LRVNPADYG, LRRNPADYG, VRLRWNPADYP, RLRVNPADY, LRVNPADYG, WIDVRLRGNPA, RLNPADY, RFNPADY, RLRLNPADY, RLRGNPADY, DVRLRINPADY, DVRLRVNPADY, WVDYNLKWNPDDY, YNLKWNPDDY, KWNPDDY, LFSHLQNEQWVDY, NEQWVDY and HLQNEQWVDY.

In a preferment, independently for each occurrence, P comprises an entire sequence selected from SEQ ID NOs: 1-100, SEQ ID NOs: 101-200 and SEQ ID NOs: 201-206, in particular from

LRRNPAD, NPADYRG, NPADYHG, VRLRWNPADYP, LRGNPAD,
WNPADYR, LRFNPAD, GSLRYNP, LRVNPADYG, LRRNPADYG,
VRLRWNPADYP, RLRVNPADY, LRVNPADYG, WIDVRLRGNPA,
RLNPADY, RFNPADY, RLRLNPADY, RLRGNPADY,
DVRLRINPADY, DVRLRVNPADY, WVDYNLKWNPDDY,
YNLKWNPDDY, KWNPDDY, LFSHLQNEQWVDY, NEQWVDY and
HLQNEQWVDY.

In a further preferred embodiment, independently for each occurrence, P comprises at least 6, preferably at least 7, more preferably at least 8, yet more preferably at least 9, even more preferably at least 10, yet even more preferably at least 11 or even 12, especially all consecutive amino acids of an amino-acid sequence selected from SEQ ID NOs: 1-50 and SEQ ID NOs: 101-150, optionally wherein at most two, preferably at most one amino acid of said amino-acid sequence is independently substituted by any other amino acid.

In a further preferred embodiment, independently for each occurrence, P comprises at least 6, preferably at least 7, more preferably at least 8, yet more preferably at least 9, even more preferably at least 10, yet even more preferably at least 11 or even 12, especially all consecutive amino acids of an amino-acid sequence selected from SEQ ID NOs: 1-20 and SEQ ID NOs: 101-120, optionally wherein at most two, preferably at most one amino acid of said amino-acid sequence is independently substituted by any other amino acid.

In a further preferred embodiment, independently for each occurrence, P comprises at least 6, preferably at least 7, more preferably at least 8, yet more preferably at least 9, even more preferably at least 10, yet even more preferably at least 11 or even 12, especially all consecutive amino acids of an amino-acid sequence selected from SEQ ID NOs: 1-10 and SEQ ID NOs: 101-110, optionally wherein at most two, preferably at most one amino acid of said amino-acid sequence is independently substituted by any other amino acid.

In yet another preferred embodiment, if P comprises at least 6 consecutive amino acids of an amino-acid sequence selected from SEQ ID NOs: 101-200 and 201-206 (optionally wherein at most two, preferably at most one amino acid of said amino-acid sequence is independently substituted by any other amino acid), P is a circularized (i.e. cyclic) peptide.

In yet another preferred embodiment, if P comprises at least 6 consecutive amino acids of an amino-acid sequence selected from SEQ ID NOs: 1-100 (optionally wherein at most two, preferably at most one amino acid of said amino-acid sequence is independently substituted by any other amino acid), P is a linear peptide.

In a further particular preference, at least one occurrence of peptide P, or peptide Pₐ and/or peptide P_{b}, (or the compound as a whole) is capable of binding anti-AChR mAb198 or anti-AChR mAb637 (as disclosed herein, see in particular Examples 14 and 15). Typically said binding of the mAb is a binding with an affinity that is lower than (i.e. “stronger than”) 1000 nM, preferably lower than 100 nM, more preferably lower than 50 nM, even more preferably lower than 10 nM, especially lower than 5 nM (especially at physiological conditions, e.g. in the bloodstream inside the human body).

It is highly preferred that the peptides used for the inventive compound do not bind to any HLA Class I or HLA Class II molecule (i.e. of the individual to be treated, e.g. human), in order to prevent presentation and stimulation via a T-cell receptor in vivo and thereby induce an immune reaction. It is generally not desired to involve any suppressive (or stimulatory) T-cell reaction in contrast to antigen-specific immunologic tolerization approaches. Therefore, to avoid T-cell epitope activity as much as possible, the peptides of the compound of the present invention (e.g. peptide P or Pₐ or P_{b}) preferably fulfil one or more of the following characteristics:
To reduce the probability for a peptide used in the compound of the present invention to bind to an HLA Class II or Class I molecule, the peptide (e.g. peptide P or Pₐ or P_{b}) has a preferred length of 4-8 amino acids, although somewhat shorter or longer lengths are still acceptable.
To further reduce the probability that such a peptide binds to an HLA Class II or Class I molecule, it is preferred to test the candidate peptide sequence by HLA binding prediction algorithms such as NetMHCII-2.3 (reviewed by Jensen et al 2018). Preferably, a peptide (e.g. peptide P or Pₐ or P_{b}) used in the compound of the present invention has (predicted) HLA binding (IC50) of at least 500 nM. More preferably, HLA binding (IC50) is more than 1000 nM, especially more than 2000 nM (cf. e.g. Peters et al 2006). In order to decrease the likelihood of HLA Class I binding, NetMHCpan 4.0 may also be applied for prediction (Jurtz et al 2017).
To further reduce the probability that such a peptide binds to an HLA Class I molecule, the NetMHCpan Rank percentile threshold can be set to a background level of 10% according to Koşalo\check{g}lu-Yalçin et al 2018. Preferably, a peptide (e.g. peptide P or Pₐ or P_{b}) used in the compound of the present invention therefore has a % Rank value of more than 3, preferably more than 5, more preferably more than 10 according to the NetMHCpan algorithm.
To further reduce the probability that such a peptide binds to an HLA Class II molecule, it is beneficial to perform in vitro HLA-binding assays commonly used in the art such as for example refolding assays, iTopia, peptide rescuing assays or array-based peptide binding assays. Alternatively, or in addition thereto, LC-MS based analytics can be used, as e.g. reviewed by Gfeller et al 2016.

For stronger reduction of the titre of the undesired antibodies, it is preferred that the peptides used in the present invention are circularized (see also Example 4). Accordingly, in a preferred embodiment, at least one occurrence of P is a circularized peptide (in particular if P comprises at least 6 consecutive amino acids, or the entire sequence, of an amino-acid sequence selected from SEQ ID NOs: 101-200 and 201-206 optionally wherein at most two, preferably at most one amino acid of said amino-acid sequence is independently substituted by any other amino acid). Preferably at least 10% of all occurrences of P are circularized peptides, more preferably at least 25% of all occurrences of P are circularized peptides, yet more preferably at least 50% of all occurrences of P are circularized peptides, even more preferably at least 75% of all occurrences of P are circularized peptides, yet even more preferably at least 90% of all occurrences of P are circularized peptides or even at least 95% of all occurrences of P are circularized peptides, especially all of the occurrences of P are circularized peptides. Several common techniques are available for circularization of peptides, see e.g. Ong et al 2017. It goes without saying that “circularized peptide” as used herein shall be understood as the peptide itself being circularized, as e.g. disclosed in Ong et al. (and not e.g. grafted on a circular scaffold with a sequence length that is longer than 13 amino acids). Such peptides may also be referred to as cyclopeptides or cyclic peptides herein.

Further, for stronger reduction of the titre of the undesired antibodies relative to the amount of scaffold used, in a preferred embodiment of the compound of the present invention, independently for each of the peptide n-mers, n is at least 2, more preferably at least 3, especially at least 4. Usually, in order to avoid complexities in the manufacturing process, independently for each of the peptide n-mers, n is less than 10, preferably less than 9, more preferably less than 8, even more preferably less than 7, yet even more preferably less than 6, especially less than 5. To benefit from higher avidity through divalent binding of the undesired antibody, it is highly preferred that, for each of the peptide n-mers, n is 2.

For multivalent binding of the undesired antibodies, it is advantageous that the peptide dimers or n-mers are spaced by a hydrophilic, structurally flexible, immunologically inert, non-toxic and clinically approved spacer such as (hetero-)bifunctional and -trifunctional Polyethylene glycol (PEG) spacers (e.g. NHS-PEG-Maleimide)—a wide range of PEG chains is available and PEG is approved by the FDA. Alternatives to PEG linkers such as immunologically inert and non-toxic synthetic polymers or glycans are also suitable. Accordingly, in the context of the present invention, the spacer (e.g. spacer S) is preferably selected from PEG molecules or glycans. For instance, the spacer such as PEG can be introduced during peptide synthesis. Such spacers (e.g. PEG spacers) may have a molecular weight of e.g. 10000 Dalton. Evidently, within the context of the present invention, the covalent binding of the peptide n-mers to the biopolymer scaffold via a linker each may for example also be achieved by binding of the linker directly to a spacer of the peptide n-mer (instead of, e.g., to a peptide of the peptide n-mer).

Preferably, each of the peptide n-mers is covalently bound to the biopolymer scaffold, preferably via a linker each.

As used herein, the linker may e.g. be selected from disulphide bridges and PEG molecules.

According to a further preferred embodiment of the inventive compound, independently for each occurrence, P is Pₐ or P_{b}.

Furthermore, it is preferred when in the first peptide n-mer, each occurrence of P is Pₐ and, in the second peptide n-mer, each occurrence of P is P_{b}. Alternatively, or in addition thereto, Pₐ and/or P_{b} is circularized.

Divalent binding is particularly suitable to reduce antibody titres. According, in a preferred embodiment,

the first peptide n-mer is Pₐ—S—Pₐ and the second peptide n-mer is Pₐ—S—Pₐ;

the first peptide n-mer is Pₐ—S—Pₐ and the second peptide n-mer is P_{b}—S—P_{b};

the first peptide n-mer is P_{b}—S—P_{b} and the second peptide n-mer is P_{b}—S—P_{b};

the first peptide n-mer is Pₐ—S—P_{b} and the second peptide n-mer is Pₐ—S—P_{b};

the first peptide n-mer is Pₐ—S—P_{b} and the second peptide n-mer is Pₐ—S—Pₐ; or

the first peptide n-mer is Pₐ—S—P_{b} and the second peptide n-mer is P_{b}—S—P_{b}.

For increasing effectivity, in a preferred embodiment the first peptide n-mer is different from the second peptide n-mer. For similar reasons, preferably, the peptide Pₐ is different from the peptide P_{b}, preferably wherein the peptide Pₐ and the peptide P_{b} are two different epitopes of the same antigen or two different epitope parts of the same epitope.

Especially for better targeting of polyclonal antibodies, it is advantageous when the peptide Pₐ and the peptide P_{b} overlap at least partially, i.e. they comprise the same amino-acid sequence fragment, wherein the amino-acid sequence fragment has a length of at least 2 amino acids, preferably at least 3 amino acids, more preferably at least 4 amino acids, yet more preferably at least 5 amino acids, even more preferably at least 6 amino acids, yet even more preferably at least 7 amino acids, especially at least 8 amino acids or even at least 9 amino acids.

Further, for stronger reduction of the titre of the undesired antibodies relative to the amount of scaffold used, the compound comprises a plurality of said first peptide n-mer (e.g. up to 10 or 20 or 30) and/or a plurality of said second peptide n-mer (e.g. up to 10 or 20 or 30).

As also illustrated above, it is highly preferred when the compound of the present invention is non-immunogenic in a mammal, preferably in a human, in a non-human primate, in a sheep, in a pig, in a dog or in a rodent.

In the context of the present invention, a non-immunogenic compound preferably is a compound wherein the biopolymer scaffold (if it is a protein) and/or the peptides (of the peptide n-mers) have an IC50 higher than 100 nM, preferably higher than 500 nM, even more preferably higher than 1000 nM, especially higher than 2000 nM, against HLA-DRB1_0101 as predicted by the NetMHCII-2.3 algorithm. The NetMHCII-2.3 algorithm is described in detail in Jensen et al, which is incorporated herein by reference. The algorithm is publicly available under http://www.cbs.dtu.dk/services/NetMHCII-2.3/. Even more preferably, a non-immunogenic compound (or pharmaceutical composition) does not bind to any HLA and/or MHC molecule (e.g. in a mammal, preferably in a human, in a non-human primate, in a sheep, in a pig, in a dog or in a rodent; or of the individual to be treated) in vivo.

According to a further preference, the compound is for intracorporeal sequestration (or intracorporeal depletion) of at least one antibody (in particular anti nicotinic AChR antibodies) in an individual, preferably in the bloodstream of the individual and/or for reduction of the titre of at least one antibody (in particular anti nicotinic AChR antibodies) in the individual, preferably in the bloodstream of the individual.

In an aspect, the present invention relates to a pharmaceutical composition comprising the inventive and at least one pharmaceutically acceptable excipient.

In embodiments, the composition is prepared for intraperitoneal, subcutaneous, intramuscular and/or intravenous administration. In particular, the composition is for repeated administration (since it is typically non-immunogenic).

In a preference, the molar ratio of peptide P or Pₐ or P_{b} to biopolymer scaffold in the composition is from 2:1 to 100:1, preferably from 3:1 to 90:1, more preferably from 4:1 to 80:1, even more preferably from 5:1 to 70:1, yet even more preferably from 6:1 to 60:1, especially from 7:1 to 50:1 or even from 8:10 to 40:1.

In another aspect, the compound of the present invention is for use in therapy.

In the course of the present invention, it turned out that the in vivo kinetics of undesirable-antibody lowering by the inventive compound is typically very fast, sometimes followed by a mild rebound of the undesirable antibody. It is thus particularly preferred when the compound (or the pharmaceutical composition comprising the compound) is administered at least twice within a 96-hour window, preferably within a 72-hour window, more preferably within a 48-hour window, even more preferably within a 36-hour window, yet even more preferably within a 24-hour window, especially within a 18-hour window or even within a 12-hour window.

In embodiments, one or more antibodies are present in the individual which are specific for at least one occurrence of peptide P, or for peptide Pₐ and/or peptide P_{b}, preferably wherein said antibodies are anti nicotinic AChR antibodies, especially anti human nicotinic AChR antibodies.

It is highly preferred that the composition is non-immunogenic in the individual (e.g. it does not comprise an adjuvant or an immunostimulatory substance that stimulates the innate or the adaptive immune system, e.g. such as an adjuvant or a T-cell epitope).

The composition of the present invention may be administered at a dose of 1-1000 mg, preferably 2-500 mg, more preferably 3-250 mg, even more preferably 4-100 mg, especially 5-50 mg, compound per kg body weight of the individual, preferably wherein the composition is administered repeatedly. Such administration may be intraperitoneally, subcutaneously, intramuscularly or intravenously.

In an aspect, the present invention relates to a method of sequestering (or depleting) one or more antibodies (preferably wherein said antibodies are anti nicotinic AChR antibodies, especially anti human nicotinic AChR antibodies) present in an individual, comprising
obtaining a pharmaceutical composition as defined herein, wherein the composition is non-immunogenic in the individual and wherein the one or more antibodies present in the individual are specific for at least one occurrence of P, or for peptide Pₐ and/or peptide P_{b}; and
administering (in particular repeatedly administering, e.g. at least two times, preferably at least three times, more preferably at least five times) the pharmaceutical composition to the individual.

In the context of the present invention, the individual (to be treated) may be a human or a non-human animal, preferably a non-human primate, a sheep, a pig, a dog or a rodent, in particular a mouse.

Preferably, the biopolymer scaffold is autologous with respect to the individual, preferably wherein the biopolymer scaffold is an autologous protein (i.e. murine albumin is used when the individual is a mouse).

In embodiments, the individual has MG (or an autoimmune channelopathy such as autoimmune autonomic ganglionopathy or Morvan syndrome, or a paraneoplastic neurological syndrome) or is at risk of developing MG (or an autoimmune channelopathy such as autoimmune autonomic ganglionopathy or Morvan syndrome, or a paraneoplastic neurological syndrome).

In general, screening for peptide mimotopes per se is known in the art, see for instance Shanmugam et al. Mimotope-based compounds of the invention have the following two advantages over compounds based on wild-type epitopes: First, the undesired antibodies, as a rule, have even higher affinities for mimotopes found by screening a peptide library, leading to higher clearance efficiency of the mimotope-based compound. Second, mimotopes further enable avoiding T-cell epitope activity as much as possible (as described hereinabove) in case the wild-type epitope sequence induces such T-cell epitope activity.

In a further aspect, the present invention relates to a peptide (preferably with a sequence length of 6 to 50 amino acids, more preferably 6 to 25 amino acids, even more preferably 6 to 20 amino acids, yet more preferably 7 to 13 amino acids), wherein the peptide is defined as disclosed herein (in particular wherein the peptide comprises P, Pₐ, or P_{b} as disclosed herein).

In certain embodiments, such peptides may be used as probes for the diagnostic typing and analysis of circulating anti nicotinic AChR antibodies, such as involved in MG. The peptides can e.g. be used as part of a diagnostic anti nicotinic AChR typing or screening device or kit or procedure, as a companion diagnostic, for patient stratification or for monitoring autoantibody levels in the course of therapeutic treatments.

In a further aspect, the invention relates to a method for detecting and/or quantifying antibodies in a biological sample comprising the steps of
bringing the sample into contact with the peptide defined as disclosed herein (in particular wherein the peptide comprises P, Pₐ, or P_{b} as disclosed herein), and
detecting the presence and/or concentration of antibodies in the sample.

The skilled person is familiar with methods for detecting and/or quantifying antibodies in biological samples. The method can e.g. be a sandwich assay, preferably an enzyme-linked immunosorbent assay (ELISA), or a surface plasmon resonance (SPR) assay.

In a preference, the peptide is immobilized on a solid support, preferably an ELISA plate or an SPR chip or a biosensor-based diagnostic device with an electrochemical, fluorescent, magnetic, electronic, gravimetric or optical biotransducer. Alternatively, or in addition thereto, the peptide may be coupled to a reporter or reporter fragment, such as a reporter fragment suitable for a protein-fragment complementation assay (PCA); see e.g. Li et al, 2019, or Kanulainen et al, 2021.

Preferably, the sample is obtained from a mammal, preferably a human. Preferably the sample is a blood sample, preferably a whole blood, serum, or plasma sample.

The invention further relates to the use of a peptide defined as disclosed herein (in particular wherein the peptide comprises P, Pₐ, or P_{b} as disclosed herein) in a diagnostic assay, preferably ELISA, preferably as disclosed herein above.

A further aspect of the invention relates to a diagnostic device comprising the peptide defined as disclosed herein (in particular wherein the peptide comprises P, Pₐ, or P_{b} as disclosed herein), preferably immobilized on a solid support. In a preference, the solid support is an ELISA plate or a surface plasmon resonance chip. In another preference, the diagnostic device is a biosensor-based diagnostic device with an electrochemical, fluorescent, magnetic, electronic, gravimetric or optical biotransducer.

In another preferred embodiment, the diagnostic device is a lateral flow assay.

The invention further relates to a diagnostic kit comprising a peptide defined as disclosed herein (in particular wherein the peptide comprises P, Pₐ, or P_{b} as disclosed herein), preferably a diagnostic device as defined herein. Preferably the diagnostic kit further comprises one or more selected from the group of a buffer, a reagent, instructions. Preferably the diagnostic kit is an ELISA kit.

A further aspect relates to an apheresis device comprising the peptide defined as disclosed herein (in particular wherein the peptide comprises P, Pₐ, or P_{b} as disclosed herein). Preferably the peptide is immobilized on a solid carrier. It is especially preferred if the apheresis device comprises at least two, preferably at least three, more preferably at least four different peptides defined as disclosed herein (in particular wherein the peptide comprises P, Pₐ, or P_{b} as disclosed herein) In a preferred embodiment the solid carrier comprises the inventive compound.

Preferably, the solid carrier is capable of being contacted with blood or plasma flow. Preferably, the solid carrier is a sterile and pyrogen-free column.

In the context of the present invention, for improved bioavailability, it is preferred that the inventive compound has a solubility in water at 25° C. of at least 0.1 μg/ml, preferably at least 1 μg/ml, more preferably at least 10 μg/ml, even more preferably at least 100 μg/ml, especially at least 1000 μg/ml.

The term “preventing” or “prevention” as used herein means to stop a disease state or condition from occurring in a patient or subject completely or almost completely or at least to a (preferably significant) extent, especially when the patient or subject or individual is predisposed to such a risk of contracting a disease state or condition.

The pharmaceutical composition of the present invention is preferably provided as a (typically aqueous) solution, (typically aqueous) suspension or (typically aqueous) emulsion. Excipients suitable for the pharmaceutical composition of the present invention are known to the person skilled in the art, upon having read the present specification, for example water (especially water for injection), saline, Ringer's solution, dextrose solution, buffers, Hank solution, vesicle forming compounds (e.g. lipids), fixed oils, ethyl oleate, 5% dextrose in saline, substances that enhance isotonicity and chemical stability, buffers and preservatives. Other suitable excipients include any compound that does not itself induce the production of antibodies in the patient (or individual) that are harmful for the patient (or individual). Examples are well tolerable proteins, polysaccharides, polylactic acids, polyglycolic acid, polymeric amino acids and amino acid copolymers. This pharmaceutical composition can (as a drug) be administered via appropriate procedures known to the skilled person (upon having read the present specification) to a patient or individual in need thereof (i.e. a patient or individual having or having the risk of developing the diseases or conditions mentioned herein). The preferred route of administration of said pharmaceutical composition is parenteral administration, in particular through intraperitoneal, subcutaneous, intramuscular and/or intravenous administration. For parenteral administration, the pharmaceutical composition of the present invention is preferably provided in injectable dosage unit form, e.g. as a solution (typically as an aqueous solution), suspension or emulsion, formulated in conjunction with the above-defined pharmaceutically acceptable excipients. The dosage and method of administration, however, depends on the individual patient or individual to be treated. Said pharmaceutical composition can be administered in any suitable dosage known from other biological dosage regimens or specifically evaluated and optimised for a given individual. For example, the active agent may be present in the pharmaceutical composition in an amount from 1 mg to 10 g, preferably 50 mg to 2 g, in particular 100 mg to 1 g. Usual dosages can also be determined on the basis of kg body weight of the patient, for example preferred dosages are in the range of 0.1 mg to 100 mg/kg body weight, especially 1 to 10 mg/kg body weight (per administration session). The administration may occur e.g. once daily, once every other day, once per week or once every two weeks. As the preferred mode of administration of the inventive pharmaceutical composition is parenteral administration, the pharmaceutical composition according to the present invention is preferably liquid or ready to be dissolved in liquid such sterile, de-ionised or distilled water or sterile isotonic phosphate-buffered saline (PBS). Preferably, 1000 μg (dry-weight) of such a composition comprises or consists of 0.1-990 μg, preferably 1-900 μg, more preferably 10-200 μg compound, and option-ally 1-500 μg, preferably 1-100 μg, more preferably 5-15 μg (buffer) salts (preferably to yield an isotonic buffer in the final volume), and optionally 0.1-999.9 μg, preferably 100-999.9 μg, more preferably 200-999 μg other excipients. Preferably, 100 mg of such a dry composition is dissolved in sterile, de-ionised/distilled water or sterile isotonic phosphate-buffered saline (PBS) to yield a final volume of 0.1-100 ml, preferably 0.5-20 ml, more preferably 1-10 ml.

It is evident to the skilled person that active agents and drugs described herein can also be administered in salt-form (i.e. as a pharmaceutically acceptable salt of the active agent). Accordingly, any mention of an active agent herein shall also include any pharmaceutically acceptable salt forms thereof.

Methods for chemical synthesis of peptides used for the compound of the present invention are well-known in the art. Of course, it is also possible to produce the peptides using recombinant methods. The peptides can be produced in microorganisms such as bacteria, yeast or fungi, in eukaryotic cells such as mammalian or insect cells, or in a recombinant virus vector such as adenovirus, poxvirus, herpesvirus, Simliki forest virus, baculovirus, bacteriophage, sindbis virus or sendai virus. Suitable bacteria for producing the peptides include E. coli, B. subtilis or any other bacterium that is capable of expressing such peptides. Suitable yeast cells for expressing the peptides of the present invention include Saccharomyces cerevisiae, Schizosaccharomyces pombe, Candida, Pichiapastoris or any other yeast capable of expressing peptides. Corresponding means and methods are well known in the art. Also, methods for isolating and purifying recombinantly produced peptides are well known in the art and include e.g. gel filtration, affinity chromatography, ion exchange chromatography etc.

Beneficially, cysteine residues are added to the peptides at the N- and/or C-terminus to facilitate coupling to the biopolymer scaffold, especially.

To facilitate isolation of said peptides, fusion polypeptides may be made wherein the peptides are translationally fused (covalently linked) to a heterologous polypeptide which enables isolation by affinity chromatography. Typical heterologous polypeptides are His-Tag (e.g. His6; 6 histidine residues), GST-Tag (Glutathione-S-transferase) etc. The fusion polypeptide facilitates not only the purification of the peptides but can also prevent the degradation of the peptides during the purification steps. If it is desired to remove the heterologous polypeptide after purification, the fusion polypeptide may comprise a cleavage site at the junction between the peptide and the heterologous polypeptide. The cleavage site may consist of an amino acid sequence that is cleaved with an enzyme specific for the amino acid sequence at the site (e.g. proteases).

The coupling/conjugation chemistry used to link the peptides/peptide n-mers to the biopolymer scaffold (e.g. via heterobifunctional compounds such as GMBS and of course also others as described in “Bioconjugate Techniques”, Greg T. Hermanson) or used to conjugate the spacer to the peptides in the context of the present invention can also be selected from reactions known to the skilled in the art. The biopolymer scaffold itself may be recombinantly produced or obtained from natural sources.

Herein, the term “specific for”—as in “molecule A specific for molecule B”—means that molecule A has a binding preference for molecule B compared to other molecules in an individual's body. Typically, this entails that molecule A (such as an antibody) has a dissociation constant (also called “affinity”) in regard to molecule B (such as the antigen, specifically the binding epitope thereof) that is lower than (i.e. “stronger than”) 1000 nM, preferably lower than 100 nM, more preferably lower than 50 nM, even more preferably lower than 10 nM, especially lower than 5 nM.

Herein, “UniProt” refers to the Universal Protein Resource. UniProt is a comprehensive resource for protein sequence and annotation data. UniProt is a collaboration between the European Bioinformatics Institute (EMBL-EBI), the SIB Swiss Institute of Bioinformatics and the Protein Information Resource (PIR). Across the three institutes more than 100 people are involved through different tasks such as database curation, software development and support. Website: http://www.uniprot.org/

Entries in the UniProt databases are identified by their accession codes (referred to herein e.g. as “UniProt accession code” or briefly as “UniProt” followed by the accession code), usually a code of six alphanumeric letters (e.g. “Q1HVF7”). If not specified otherwise, the accession codes used herein refer to entries in the Protein Knowledgebase (UniProtKB) of UniProt. If not stated otherwise, the UniProt database state for all entries referenced herein is of 13 Feb. 2019 (UniProt/UniProtKB Release 2019_02).

In the context of the present application, sequence variants (designated as “natural variant” in UniProt) are expressly included when referring to a UniProt database entry.

The present invention further relates to the following embodiments:

Embodiment 1. A compound comprising
a biopolymer scaffold and at least
a first peptide n-mer of the general formula:

P(—S—P)₍ₙ₋₁₎ and
a second peptide n-mer of the general formula:

P(—S—P)₍ₙ₋₁₎;

wherein, independently for each occurrence, P is a peptide with a sequence length of 6-13 (preferably 7-13, especially 8-13) amino acids, and S is a non-peptide spacer,

wherein, independently for each of the peptide n-mers, n is an integer of at least 1, preferably of at least 2, more preferably of at least 3, especially of at least 4,

wherein each of the peptide n-mers is bound to the biopolymer scaffold, preferably via a linker each, wherein, independently for each occurrence, P comprises at least 6, preferably at least 7, more preferably at least 8, yet more preferably at least 9, even more preferably at least 10, yet even more preferably at least 11 or even 12, especially all consecutive amino acids of a (preferably human nicotinic AChR MIR-derived) amino-acid sequence selected from SEQ ID NOs: 1-100, SEQ ID NOs: 101-200 and SEQ ID NOs: 201-206,

optionally wherein at most two, preferably at most one amino acid of said amino-acid sequence is independently substituted by any other amino acid.

Embodiment 2. The compound of embodiment 1, wherein at least one occurrence of P is a circularized peptide (in particular if P comprises at least 6 consecutive amino acids, or the entire sequence, of an amino-acid sequence selected from SEQ ID NOs: 101-200 and 201-206 optionally wherein at most two, preferably at most one amino acid of said amino-acid sequence is independently substituted by any other amino acid), preferably wherein at least 10% of all occurrences of P are circularized peptides, more preferably wherein at least 25% of all occurrences of P are circularized peptides, yet more preferably wherein at least 50% of all occurrences of P are circularized peptides, even more preferably wherein at least 75% of all occurrences of P are circularized peptides, yet even more preferably wherein at least 90% of all occurrences of P are circularized peptides or even wherein at least 95% of all occurrences of P are circularized peptides, especially wherein all of the occurrences of P are circularized peptides.
Embodiment 3. The compound of embodiment 1 or 2, wherein, independently for each of the peptide n-mers, n is at least 2, more preferably at least 3, especially at least 4.
Embodiment 4. The compound of any one of embodiments 1 to 3, wherein, independently for each of the peptide n-mers, n is less than 10, preferably less than 9, more preferably less than 8, even more preferably less than 7, yet even more preferably less than 6, especially less than 5.
Embodiment 5. The compound of any one of embodiments 1 to 4, wherein, for each of the peptide n-mers, n is 2.
Embodiment 6. The compound of any one of embodiments 1 to 5, wherein at least one occurrence of P is Pₐ and/or at least one occurrence of P is P_{b},

wherein Pₐ is a peptide with a sequence length of 6-13 amino acids, preferably 7-13 amino acids, more preferably 7-11 amino acids,

wherein P_{b} is a peptide with a sequence length of 6-13 amino acids, preferably 7-13 amino acids, more preferably 7-11 amino acids.

Embodiment 7. The compound of any one of embodiments 1 to 6, wherein, independently for each occurrence, P is Pₐ or P_{b}.
Embodiment 8. The compound of any one of embodiments 1 to 7, wherein, in the first peptide n-mer, each occurrence of P is Pₐ and, in the second peptide n-mer, each occurrence of P is P_{b}.
Embodiment 9. The compound of any one of embodiments 1 to 8, wherein

the first peptide n-mer is Pₐ—S—Pₐ and the second peptide n-mer is Pₐ—S—Pₐ; or

the first peptide n-mer is Pₐ—S—Pₐ and the second peptide n-mer is P_{b}—S—P_{b};

the first peptide n-mer is P_{b}—S—P_{b} and the second peptide n-mer is P_{b}—S—P_{b};

the first peptide n-mer is Pₐ—S—P_{b} and the second peptide n-mer is Pₐ—S—P_{b};

the first peptide n-mer is Pₐ—S—P_{b} and the second peptide n-mer is Pₐ—S—Pₐ; or

the first peptide n-mer is Pₐ—S—P_{b} and the second peptide n-mer is P_{b}—S—P_{b}.

Embodiment 10. A compound comprising
a biopolymer scaffold and at least
a first peptide n-mer which is a peptide dimer of the formula Pₐ—S—Pₐ or Pₐ—S—P_{b},

wherein Pₐ is a peptide with a sequence length of 6-13 amino acids, preferably 7-13 amino acids, more preferably 7-11 amino acids, P_{b} is a peptide with a sequence length of 6-13 amino acids, preferably 7-13 amino acids, more preferably 7-11 amino acids, and S is a non-peptide spacer,

wherein the first peptide n-mer is bound to the biopolymer scaffold, preferably via a linker,

wherein Pₐ comprises at least 6, preferably at least 7, more preferably at least 8, yet more preferably at least 9, even more preferably at least 10, yet even more preferably at least 11 or even 12, especially all consecutive amino acids of a (preferably human nicotinic AChR MIR-derived) amino-acid sequence selected from SEQ ID NOs: 1-100, SEQ ID NOs: 101-200 and SEQ ID NOs: 201-206, preferably SEQ ID NOs: 1-50, SEQ ID NOs: 101-150 and SEQ ID NOs: 201-206, especially SEQ ID NOs: 1-20, SEQ ID NOs: 101-20 and SEQ ID NOs: 201-206; optionally wherein at most two, preferably at most one amino acid of said amino-acid sequence is independently substituted by any other amino acid.

Embodiment 11. The compound of embodiment 10, further comprising a second peptide n-mer which is a peptide dimer of the formula P_{b}—S—P_{b} or Pₐ—S—P_{b},

wherein the second peptide n-mer is bound to the biopolymer scaffold, preferably via a linker,

wherein P_{b} comprises at least 6, preferably at least 7, more preferably at least 8, yet more preferably at least 9, even more preferably at least 10, yet even more preferably at least 11 or even 12, especially all consecutive amino acids of a (preferably human nicotinic AChR MIR-derived) amino-acid sequence selected from SEQ ID NOs: 1-100, SEQ ID NOs: 101-200 and SEQ ID NOs: 201-206, preferably SEQ ID NOs: 1-50, SEQ ID NOs: 101-150 and SEQ ID NOs: 201-206, especially SEQ ID NOs: 1-20, SEQ ID NOs: 101-20 and SEQ ID NOs: 201-206; optionally wherein at most two, preferably at most one amino acid of said amino-acid sequence is independently substituted by any other amino acid

Embodiment 13. The compound of any one of embodiments 6 to 12, wherein the peptide Pₐ is different from the peptide P_{b}.
Embodiment 14. The compound of any one of embodiments 6 to 13, wherein the peptide Pₐ and the peptide P_{b} comprise the same amino-acid sequence fragment, wherein said amino-acid sequence fragment has a length of at least 2 amino acids, preferably at least 3 amino acids, more preferably at least 4 amino acids, yet more preferably at least 5 amino acids, even more preferably at least 6 amino acids, yet even more preferably at least 7 amino acids, especially at least 8 amino acids or even at least 9 amino acids.
Embodiment 15. The compound of any one of embodiments 6 to 14, wherein Pₐ and/or P_{b} is circularized, in particular if Pₐ and/or P_{b} comprises at least 6 consecutive amino acids, or the entire sequence, of an amino-acid sequence selected from SEQ ID NOs: 101-200 and 201-206 optionally wherein at most two, preferably at most one amino acid of said amino-acid sequence is independently substituted by any other amino acid.
Embodiment 16. The compound of any one of embodiments 1 to 15, wherein the compound comprises a plurality of said first peptide n-mer and/or a plurality of said second peptide n-mer.
Embodiment 17. The compound of any one of embodiments 1 to 16, wherein the biopolymer scaffold is a protein, preferably a mammalian protein such as a human protein, a non-human primate protein, a sheep protein, a pig protein, a dog protein or a rodent protein.
Embodiment 18. The compound of embodiment 17, wherein the biopolymer scaffold is a globulin.
Embodiment 19. The compound of embodiment 18, wherein the biopolymer scaffold is selected from the group consisting of immunoglobulins, alpha1-globulins, alpha2-globulins and beta-globulins.
Embodiment 20. The compound of embodiment 19, wherein the biopolymer scaffold is selected from the group consisting of immunoglobulin G, haptoglobin and transferrin.
Embodiment 21. The compound of embodiment 20, wherein the biopolymer scaffold is haptoglobin.
Embodiment 22. The compound of embodiment 17, wherein the biopolymer scaffold is an albumin.
Embodiment 23. The compound of any one of embodiments 1 to 22, wherein the compound is non-immunogenic in a mammal, preferably in a human, in a non-human primate, in a sheep, in a pig, in a dog or in a rodent.
Embodiment 24. The compound of any one of embodiments 1 to 23, wherein the compound is for intracorporeal sequestration (or intracorporeal depletion) of at least one antibody (in particular anti nicotinic AChR antibodies) in an individual, preferably in the bloodstream of the individual and/or for reduction of the titre of at least one antibody (in particular anti nicotinic AChR antibodies) in the individual, preferably in the bloodstream of the individual.
Embodiment 25. The compound of any one of embodiments 1 to 24, wherein said amino sequence is selected from LRRNPAD, NPADYRG, NPADYHG, VRLRWNPADYP, LRGNPAD, WNPADYR, LRFNPAD, GSLRYNP, LRVNPADYG, LRRNPADYG, VRLRWNPADYP, RLRVNPADY, LRVNPADYG, WIDVRLRGNPA, RLNPADY, RFNPADY, RLRLNPADY, RLRGNPADY, DVRLRINPADY, DVRLRVNPADY, WVDYNLKWNPDDY, YNLKWNPDDY, KWNPDDY, LFSHLQNEQWVDY, NEQWVDY and HLQNEQWVDY.
Embodiment 26. The compound of any one of embodiments 1 to 25, wherein, independently for each occurrence, P comprises at least 6, preferably at least 7, more preferably at least 8, yet more preferably at least 9, even more preferably at least 10, yet even more preferably at least 11 or even 12, especially all consecutive amino acids of an amino-acid sequence selected from SEQ ID NOs: 1-50 and SEQ ID NOs: 101-150, optionally wherein at most two, preferably at most one amino acid of said amino-acid sequence is independently substituted by any other amino acid.
Embodiment 27. The compound of any one of embodiments 1 to 26, wherein, independently for each occurrence, P comprises at least 6, preferably at least 7, more preferably at least 8, yet more preferably at least 9, even more preferably at least 10, yet even more preferably at least 11 or even 12, especially all consecutive amino acids of an amino-acid sequence selected from SEQ ID NOs: 1-20 and SEQ ID NOs: 101-120, optionally wherein at most two, preferably at most one amino acid of said amino-acid sequence is independently substituted by any other amino acid.
Embodiment 28. The compound of any one of embodiments 1 to 27, wherein, independently for each occurrence, P comprises an entire sequence selected from SEQ ID NOs: 1-100, SEQ ID NOs: 101-200 and SEQ ID NOs: 201-206, in particular from LRRNPAD, NPADYRG, NPADYHG, VRLRWNPADYP, LRGNPAD, WNPADYR, LRFNPAD, GSLRYNP, LRVNPADYG, LRRNPADYG, VRLRWNPADYP, RLRVNPADY, LRVNPADYG, WIDVRLRGNPA, RLNPADY, RFNPADY, RLRLNPADY, RLRGNPADY, DVRLRINPADY, DVRLRVNPADY, WVDYNLKWNPDDY, YNLKWNPDDY, KWNPDDY, LFSHLQNEQWVDY, NEQWVDY and HLQNEQWVDY.
Embodiment 29. The compound of any one of embodiments 1 to 28, wherein at least one occurrence of P comprises at least 6 consecutive amino acids of an amino-acid sequence selected from SEQ ID NOs: 101-200 and 201-206 (optionally wherein at most two, preferably at most one amino acid of said amino-acid sequence is independently substituted by any other amino acid) and is a circularized peptide.
Embodiment 30. The compound of any one of embodiments 1 to 29, wherein at least one occurrence of P comprises at least 6 consecutive amino acids of an amino-acid sequence selected from SEQ ID NOs: 1-100 (optionally wherein at most two, preferably at most one amino acid of said amino-acid sequence is independently substituted by any other amino acid) and is a linear peptide.
Embodiment 31. The compound of any one of embodiments 1 to 30, wherein, independently for each occurrence, P consists of an entire sequence selected from SEQ ID NOs: 1-100, SEQ ID NOs: 101-200 and SEQ ID NOs: 201-206, in particular from LRRNPAD, NPADYRG, NPADYHG, VRLRWNPADYP, LRGNPAD, WNPADYR, LRFNPAD, GSLRYNP, LRVNPADYG, LRRNPADYG, VRLRWNPADYP, RLRVNPADY, LRVNPADYG, WIDVRLRGNPA, RLNPADY, RFNPADY, RLRLNPADY, RLRGNPADY, DVRLRINPADY, DVRLRVNPADY, WVDYNLKWNPDDY, YNLKWNPDDY, KWNPDDY, LFSHLQNEQWVDY, NEQWVDY and HLQNEQWVDY, optionally wherein at most two, preferably at most one amino acid of said entire sequence is independently substituted by any other amino acid.
Embodiment 32. The compound of any one of embodiments 1 to 31, wherein, independently for each occurrence, P consists of an entire sequence selected from SEQ ID NOs: 1-100, SEQ ID NOs: 101-200 and SEQ ID NOs: 201-206, in particular from LRRNPAD, NPADYRG, NPADYHG, VRLRWNPADYP, LRGNPAD, WNPADYR, LRFNPAD, GSLRYNP, LRVNPADYG, LRRNPADYG, VRLRWNPADYP, RLRVNPADY, LRVNPADYG, WIDVRLRGNPA, RLNPADY, RFNPADY, RLRLNPADY, RLRGNPADY, DVRLRINPADY, DVRLRVNPADY, WVDYNLKWNPDDY, YNLKWNPDDY, KWNPDDY, LFSHLQNEQWVDY, NEQWVDY and HLQNEQWVDY, optionally with an N-terminal and/or C-terminal cysteine residue.
Embodiment 33. The compound of any one of embodiments 1 to 32, wherein the biopolymer scaffold is a human protein or a humanized protein such as a humanized antibody.
Embodiment 34. The compound of any one of embodiments 1 to 33, wherein each of the peptide n-mers is covalently bound to the biopolymer scaffold, preferably via a linker each.
Embodiment 35. The compound of any one of embodiments 1 to 34, wherein at least one of said linkers is selected from disulphide bridges and PEG molecules.
Embodiment 36. The compound of any one of embodiments 1 to 35, wherein at least one of the spacers S is selected from PEG molecules or glycans.
Embodiment 37. The compound of any one of embodiments 1 to 36, wherein Pₐ comprises at least 6, preferably at least 7, more preferably at least 8, yet more preferably at least 9, even more preferably at least 10, yet even more preferably at least 11 or even 12, especially all consecutive amino acids of a (preferably human nicotinic AChR MIR-derived) amino-acid sequence selected from SEQ ID NOs: 1-100, SEQ ID NOs: 101-200 and SEQ ID NOs: 201-206, in particular from LRRNPAD, NPADYRG, NPADYHG, VRLRWNPADYP, LRGNPAD, WNPADYR, LRFNPAD, GSLRYNP, LRVNPADYG, LRRNPADYG, VRLRWNPADYP, RLRVNPADY, LRVNPADYG, WIDVRLRGNPA, RLNPADY, RFNPADY, RLRLNPADY, RLRGNPADY, DVRLRINPADY, DVRLRVNPADY, WVDYNLKWNPDDY, YNLKWNPDDY, KWNPDDY, LFSHLQNEQWVDY, NEQWVDY and HLQNEQWVDY, optionally wherein at most two, preferably at most one amino acid of said amino-acid sequence is independently substituted by any other amino acid.
Embodiment 38. The compound of any one of embodiments 1 to 37, wherein P_{b} comprises at least 6, preferably at least 7, more preferably at least 8, yet more preferably at least 9, even more preferably at least 10, yet even more preferably at least 11 or even 12, especially all consecutive amino acids of a (preferably human nicotinic AChR MIR-derived) amino-acid sequence selected from SEQ ID NOs: 1-100, SEQ ID NOs: 101-200 and SEQ ID NOs: 201-206, in particular from LRRNPAD, NPADYRG, NPADYHG, VRLRWNPADYP, LRGNPAD, WNPADYR, LRFNPAD, GSLRYNP, LRVNPADYG, LRRNPADYG, VRLRWNPADYP, RLRVNPADY, LRVNPADYG, WIDVRLRGNPA, RLNPADY, RFNPADY, RLRLNPADY, RLRGNPADY, DVRLRINPADY, DVRLRVNPADY, WVDYNLKWNPDDY, YNLKWNPDDY, KWNPDDY, LFSHLQNEQWVDY, NEQWVDY and HLQNEQWVDY, optionally wherein at most two, preferably at most one amino acid of said amino-acid sequence is independently substituted by any other amino acid.
Embodiment 39. The compound of any one of embodiments 1 to 36, wherein Pₐ consists of an entire sequence selected from SEQ ID NOs: 1-100, SEQ ID NOs: 101-200 and SEQ ID NOs: 201-206, in particular from LRRNPAD, NPADYRG, NPADYHG, VRLRWNPADYP, LRGNPAD, WNPADYR, LRFNPAD, GSLRYNP, LRVNPADYG, LRRNPADYG, VRLRWNPADYP, RLRVNPADY, LRVNPADYG, WIDVRLRGNPA, RLNPADY, RFNPADY, RLRLNPADY, RLRGNPADY, DVRLRINPADY, DVRLRVNPADY, WVDYNLKWNPDDY, YNLKWNPDDY, KWNPDDY, LFSHLQNEQWVDY, NEQWVDY and HLQNEQWVDY, optionally wherein at most two, preferably at most one amino acid of said entire sequence is independently substituted by any other amino acid.
Embodiment 40. The compound of any one of embodiments 1 to 37, wherein P_{b} consists of an entire sequence selected from SEQ ID NOs: 1-100, SEQ ID NOs: 101-200 and SEQ ID NOs: 201-206, in particular from LRRNPAD, NPADYRG, NPADYHG, VRLRWNPADYP, LRGNPAD, WNPADYR, LRFNPAD, GSLRYNP, LRVNPADYG, LRRNPADYG, VRLRWNPADYP, RLRVNPADY, LRVNPADYG, WIDVRLRGNPA, RLNPADY, RFNPADY, RLRLNPADY, RLRGNPADY, DVRLRINPADY, DVRLRVNPADY, WVDYNLKWNPDDY, YNLKWNPDDY, KWNPDDY, LFSHLQNEQWVDY, NEQWVDY and HLQNEQWVDY, optionally wherein at most two, preferably at most one amino acid of said entire sequence is independently substituted by any other amino acid.
Embodiment 41. The compound of any one of embodiments 6 to 40, wherein the first peptide n-mer is Pₐ—S—P_{b} and the second peptide n-mer is Pₐ—S—P_{b}.
Embodiment 42. The compound of any one of embodiments 6 to 41, wherein the peptide Pₐ and the peptide P_{b} comprise the same amino-acid sequence fragment, wherein said same amino-acid sequence fragment has a length of at least 5 amino acids, even more preferably at least 6 amino acids, yet even more preferably at least 7 amino acids, especially at least 8 amino acids or even at least 9 amino acids.
Embodiment 43. The compound of any one of embodiments 1 to 42, wherein the compound has a solubility in water at 25° C. of at least 0.1 μg/ml.
Embodiment 44. The compound of any one of embodiments 1 to 43, wherein the compound has a solubility in water at 25° C. of at least 1 μg/ml.
Embodiment 45. The compound of any one of embodiments 1 to 44, wherein the compound has a solubility in water at 25° C. of at least 10 μg/ml, preferably at least 100 μg/ml, especially at least 1000 μg/ml.
Embodiment 46. The compound of any one of embodiments 1 to 45, wherein at least one occurrence of peptide P, or peptide Pₐ and/or peptide P_{b}, (or the compound as a whole) is capable of binding anti-AChR mAb198 or anti-AChR mAb637 (as disclosed herein, see in particular Examples 14 and 15).
Embodiment 47. The compound of embodiments 1 to 46, wherein the first peptide n-mer is Pₐ—S—P_{b} and the second peptide n-mer is Pₐ—S—P_{b}.
Embodiment 48. The compound of embodiments 1 to 47, wherein the peptide Pₐ and the peptide P_{b} comprise the same amino-acid sequence fragment, wherein the amino-acid sequence fragment has a length of at least 5 amino acids, even more preferably at least 6 amino acids, yet even more preferably at least 7 amino acids, especially at least 8 amino acids or even at least 9 amino acids.
Embodiment 49. The compound of any one of embodiments 1 to 48, wherein the biopolymer scaffold is human transferrin.
Embodiment 50. The compound of any one of embodiments 1 to 49, wherein the biopolymer scaffold is an anti-CD163 antibody (i.e. an antibody specific for a CD163 protein) or CD163-binding fragment thereof.
Embodiment 51. The compound of embodiment 50, wherein the anti-CD163 antibody or CD163-binding fragment thereof is specific for human CD163 and/or is specific for the extracellular region of CD163, preferably for an SRCR domain of CD163, more preferably for any one of SRCR domains 1-9 of CD163, even more preferably for any one of SRCR domains 1-3 of CD163, especially for SRCR domain 1 of CD163.
Embodiment 52. The compound of embodiment 50 or 51, wherein the anti-CD163 antibody or CD1.63-binding fragment thereof is specific for one of the following peptides:
a peptide consisting of 7-25, preferably 8-20, even more preferably 9-15, especially 10-13 amino acids, wherein the peptide comprises the amino acid sequence CSRVEVKVQEEWGTVCNTNGWSMEA or a 7-24 amino-acid fragment thereof,
a peptide consisting of 7-25, preferably 8-20, even more preferably 9-15, especially 10-13 amino acids, wherein the peptide comprises the amino acid sequence DHVSCRCNESALWDCKHDGWG or a 7-20 amino-acid fragment thereof, Or
a peptide consisting of 7-25, preferably 8-20, even more preferably 9-15, especially 10-13 amino acids, wherein the peptide comprises the amino acid sequence SSLGGTDKELRLVDGENKCS or a 7-19 amino-acid fragment thereof.
   Embodiment 53. The compound of embodiment 50 or 51, wherein the anti-CD163 antibody or CD163-binding fragment thereof is specific for a peptide comprising the amino acid sequence ESALW or ALW.
   Embodiment 54. The compound of embodiment 50 or 51, wherein the anti-CD163 antibody or CD163-binding fragment thereof is specific for a peptide comprising the amino acid sequence GRVEVKVQEEW, WGTVCNNGWS or WGTVCNNGW.
   Embodiment 55. The compound of embodiment 50 or 51, wherein the anti-CD163 antibody or CD163-binding fragment thereof is specific for a peptide comprising the amino acid sequence SSLGGTDKELR or SSLGG.
   Embodiment 56. The compound of any one of embodiments 1 to 55, wherein said amino-acid sequence is selected from SEQ ID NOs: 1-50 and SEQ ID NOs: 101-150.
   Embodiment 57. The compound of any one of embodiments 1 to 56, wherein said amino-acid sequence is selected from SEQ ID NOs: 1-20 and SEQ ID NOs: 101-120.
   Embodiment 58. The compound of any one of embodiments 1 to 57, wherein said amino-acid sequence is selected from SEQ ID NOs: 201-206.
   Embodiment 59. The compound of any one of embodiments 1 to 58, wherein the peptides do not do not bind to any HLA Class I or HLA Class II molecule.
   Embodiment 60. The compound of any one of embodiments 1 to 59, wherein each of the peptide n-mers is covalently bound to the biopolymer scaffold via a linker each.
   Embodiment 61. The compound of any one embodiments 1 to 60, wherein the biopolymer scaffold is selected from human immunoglobulins and human transferrin.
   Embodiment 62. The compound of embodiment any one of embodiments 1 to 61, wherein the biopolymer scaffold is human transferrin.
   Embodiment 63. The compound of any one of embodiments 49 to 62, wherein at least one of the at least two peptides is circularized.
   Embodiment 64. The compound of any one of embodiments 1 to 63, wherein the compound is non-immunogenic in humans.
   Embodiment 65. A pharmaceutical composition comprising the compound of any one of embodiments 1 to 64 and at least one pharmaceutically acceptable excipient.
   Embodiment 66. The pharmaceutical composition of embodiment 65, wherein the composition is prepared for intraperitoneal, subcutaneous, intramuscular and/or intravenous administration and/or wherein the composition is for repeated administration.
   Embodiment 67. The pharmaceutical composition of any one of embodiments 1 to 66, wherein the molar ratio of peptide P to biopolymer scaffold in the composition is from 2:1 to 100:1, preferably from 3:1 to 90:1, more preferably from 4:1 to 80:1, even more preferably from 5:1 to 70:1, yet even more preferably from 6:1 to 60:1, especially from 7:1 to 50:1 or even from 8:10 to 40:1.
   Embodiment 68. The pharmaceutical composition of any one of embodiments 6 to 67, wherein the molar ratio of peptide Pₐ to biopolymer scaffold in the composition is from 2:1 to 100:1, preferably from 3:1 to 90:1, more preferably from 4:1 to 80:1, even more preferably from 5:1 to 70:1, yet even more preferably from 6:1 to 60:1, especially from 7:1 to 50:1 or even from 8:10 to 40:1.
   Embodiment 69. The pharmaceutical composition of any one of embodiments 6 to 68, wherein the molar ratio of peptide P_{b} to biopolymer scaffold in the composition is from 2:1 to 100:1, preferably from 3:1 to 90:1, more preferably from 4:1 to 80:1, even more preferably from 5:1 to 70:1, yet even more preferably from 6:1 to 60:1, especially from 7:1 to 50:1 or even from 8:10 to 40:1.
   Embodiment 70. The pharmaceutical composition of any one of embodiments 65 to 69 for use in therapy.
   Embodiment 71. The pharmaceutical composition for use according to embodiment 70, for use in prevention or treatment of MG, in particular transient neonatal MG, or of an autoimmune channelopathy such as autoimmune autonomic ganglionopathy or Morvan syndrome, or of a paraneoplastic neurological syndrome in an individual.
   Embodiment 72. The pharmaceutical composition for use according to embodiment 71, wherein the pharmaceutical composition is administered at least twice within a 96-hour window, preferably within a 72-hour window, more preferably within a 48-hour window, even more preferably within a 36-hour window, yet even more preferably within a 24-hour window, especially within a 18-hour window or even within a 12-hour window.
   Embodiment 73. The pharmaceutical composition for use according to embodiment 70, for use in sequestering one or more autoantibodies present in an individual with MG (or an autoimmune channelopathy such as autoimmune autonomic ganglionopathy or Morvan syndrome, or a paraneoplastic neurological syndrome).
   Embodiment 74. The pharmaceutical composition for use according to embodiment 73, wherein the pharmaceutical composition is administered at least twice within a 96-hour window, preferably within a 72-hour window, more preferably within a 48-hour window, even more preferably within a 36-hour window, yet even more preferably within a 24-hour window, especially within a 18-hour window or even within a 12-hour window.
   Embodiment 75. The pharmaceutical composition for use according to any one of embodiments 71 to 74, wherein the individual is human.
   Embodiment 76. The pharmaceutical composition for use according to any one of embodiments 70 to 75, wherein one or more antibodies are present in the individual which are specific for at least one occurrence of peptide P, or for peptide Pₐ and/or peptide P_{b}, preferably wherein said antibodies are autoantibodies.
   Embodiment 77. The pharmaceutical composition for use according to any one of embodiments 70 to 76, wherein the composition is non-immunogenic in the individual.
   Embodiment 78. The pharmaceutical composition for use according to any one of embodiments 70 to 77, wherein the composition is administered at a dose of 1-1000 mg, preferably 2-500 mg, more preferably 3-250 mg, even more preferably 4-100 mg, especially 5-50 mg, compound per kg body weight of the individual.
   Embodiment 79. The pharmaceutical composition for use according to any one of embodiments 70 to 78, wherein the composition is administered intraperitoneally, subcutaneously, intramuscularly or intravenously.
   Embodiment 80. A method of ameliorating or treating MG (or an autoimmune channelopathy such as autoimmune autonomic ganglionopathy or Morvan syndrome, or a paraneoplastic neurological syndrome) in an individual in need thereof, comprising
obtaining a pharmaceutical composition as defined in any one of embodiments 65 to 79; and administering an effective amount of the pharmaceutical composition to the individual.
   Embodiment 81. A method of sequestering (or depleting) one or more antibodies present in an individual, comprising
obtaining a pharmaceutical composition as defined in any one of embodiments 65 to 79, wherein the composition is non-immunogenic in the individual and wherein the one or more antibodies present in the individual are specific for at least one occurrence of P, or for peptide Pₐ and/or peptide P_{b}; and
administering the pharmaceutical composition to the individual.
   Embodiment 82. The method of embodiment 81, wherein the individual is a human or a non-human animal, preferably a non-human primate, a sheep, a pig, a dog or a rodent, in particular a mouse.
   Embodiment 83. The method of embodiments 81 or 82, wherein the biopolymer scaffold is autologous with respect to the individual, preferably wherein the biopolymer scaffold is an autologous protein.
   Embodiment 84. A peptide with a sequence length of 6 to 50 amino acids, more preferably 6 to 25 amino acids, even more preferably 6 to 20 amino acids, yet more preferably 7 to 13 amino acids, wherein the peptide comprises P, Pₐ or P_{b} as defined in any one of embodiments 81-83 or comprises at least 6, preferably at least 7, more preferably at least 8, yet more preferably at least 9, even more preferably at least 10, yet even more preferably at least 11 or even 12, especially all consecutive amino acids of a (preferably human nicotinic AChR MIR-derived) amino-acid sequence selected from SEQ ID NOs: 1-100, SEQ ID NOs: 101-200 and SEQ ID NOs: 201-206, preferably SEQ ID NOs: 1-50, SEQ ID NOs: 101-150 and SEQ ID NOs: 201-206, especially SEQ ID NOs: 1-20, SEQ ID NOs: 101-20 and SEQ ID NOs: 201-206; optionally wherein at most two, preferably at most one amino acid of said amino-acid sequence is independently substituted by any other amino acid.
   Embodiment 85. A method for detecting and/or quantifying anti-AChR antibodies in a biological sample comprising the steps of
bringing the sample into contact with the peptide of embodiment 84, and
detecting the presence and/or concentration of the antibodies in the sample.
   Embodiment 86. The method of embodiment 85, wherein the peptide is immobilized on a solid support, in particular a biosensor-based diagnostic device with an electrochemical, fluorescent, magnetic, electronic, gravimetric or optical biotransducer and/or wherein the peptide is coupled to a reporter or reporter fragment, such as a reporter fragment suitable for a PCA.
   Embodiment 87. The method of embodiment 85 or 86, wherein the method is a sandwich assay, preferably an enzyme-linked immunosorbent assay (ELISA).
   Embodiment 88. The method of any one of embodiments 85 to 87, wherein the sample is obtained from a mammal, preferably a human (preferably wherein the mammal has MG or is suspected of having MG).
   Embodiment 89. The method of any one of embodiment 85 to 88, wherein the sample is a blood sample, preferably whole blood, serum, or plasma.
   Embodiment 90. Use of the peptide according to embodiment 84 in an enzyme-linked immunosorbent assay (ELISA), preferably for a method as defined in any one of embodiments 85 to 88.
   Embodiment 91. Diagnostic device comprising the peptide according to embodiment 84, wherein the peptide is immobilized on a solid support and/or wherein the peptide is coupled to a reporter or reporter fragment, such as a reporter fragment suitable for a PCA.
   Embodiment 92. Diagnostic device according to embodiment 91, wherein the solid support is an ELISA plate or a surface plasmon resonance chip.
   Embodiment 93. Diagnostic device according to embodiment 91, wherein the diagnostic device is a lateral flow assay device or a biosensor-based diagnostic device with an electrochemical, fluorescent, magnetic, electronic, gravimetric or optical biotransducer.
   Embodiment 94. A diagnostic kit comprising a peptide according to embodiment 84, preferably diagnostic device according to any one of embodiment 91 to 93, and preferably one or more selected from the group of a buffer, a reagent, instructions.
   Embodiment 95. An apheresis device comprising the peptide according to embodiment 84, preferably immobilized on a solid carrier.
   Embodiment 96. The apheresis device according to embodiment 95, wherein the solid carrier is capable of being contacted with blood or plasma flow.
   Embodiment 97. The apheresis device according to embodiment 95 or 96, wherein the solid carrier comprises the compound according to any one of embodiments 1 to 64.
   Embodiment 98. The apheresis device according to any one of embodiment 95 to 97, wherein the solid carrier is a sterile and pyrogen-free column.
   Embodiment 99. The apheresis device according to any one of embodiments 95 to 98, wherein the apheresis device comprises at least two, preferably at least three, more preferably at least four different peptides according to embodiment 84.

The present invention is further illustrated by the following figures and examples, without being restricted thereto. In the context of the following figures and examples the compound on which the inventive approach is based is also referred to as “Selective Antibody Depletion Compound” (SADC).

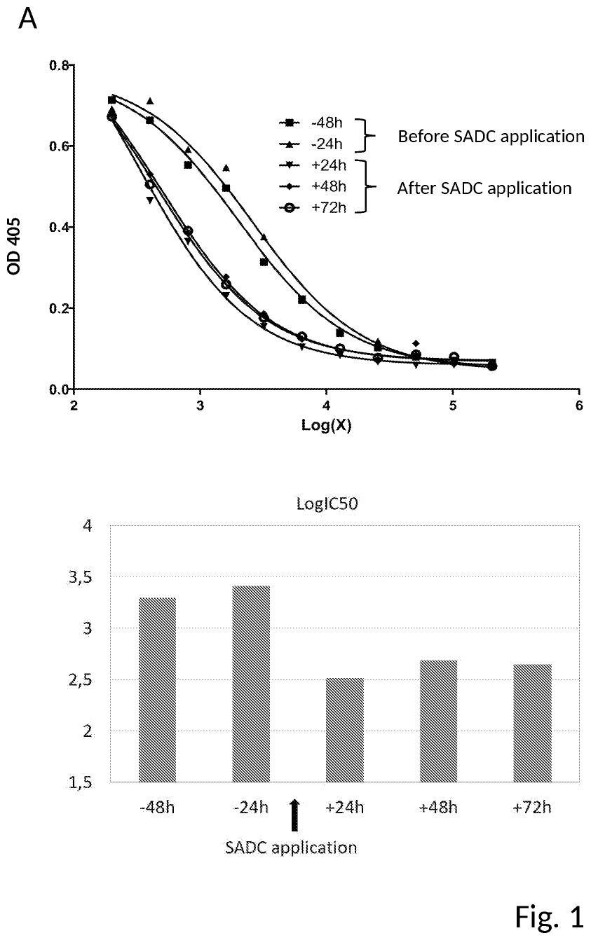

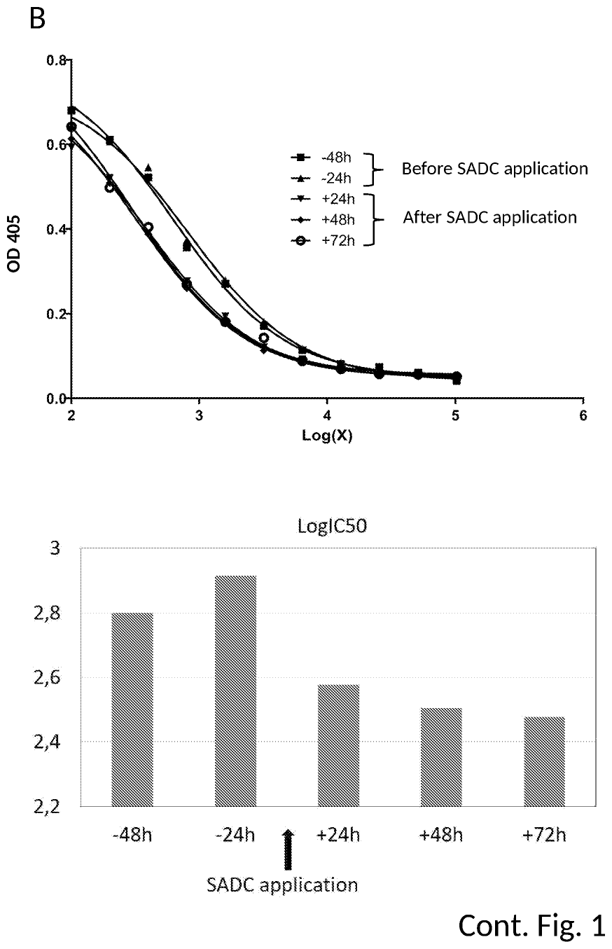

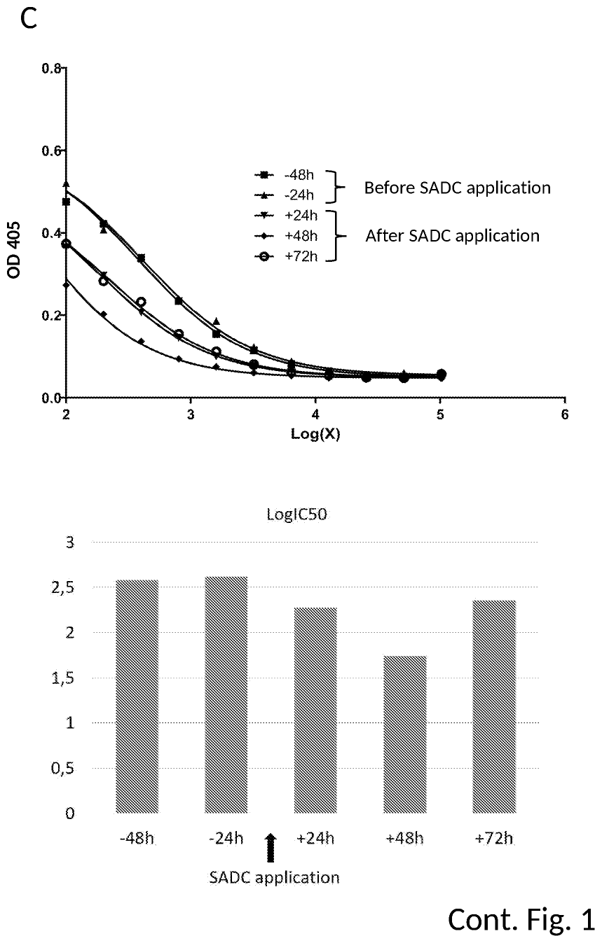

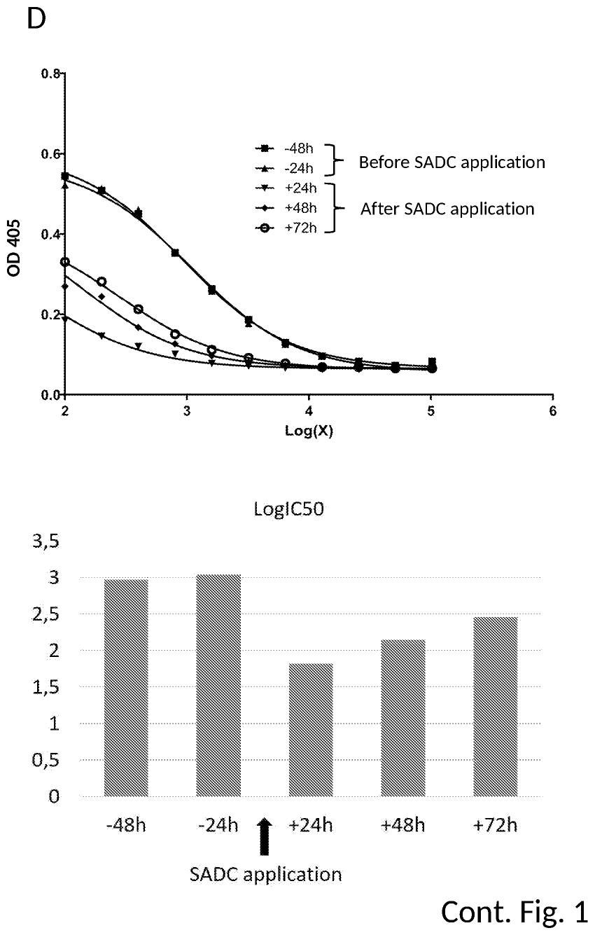

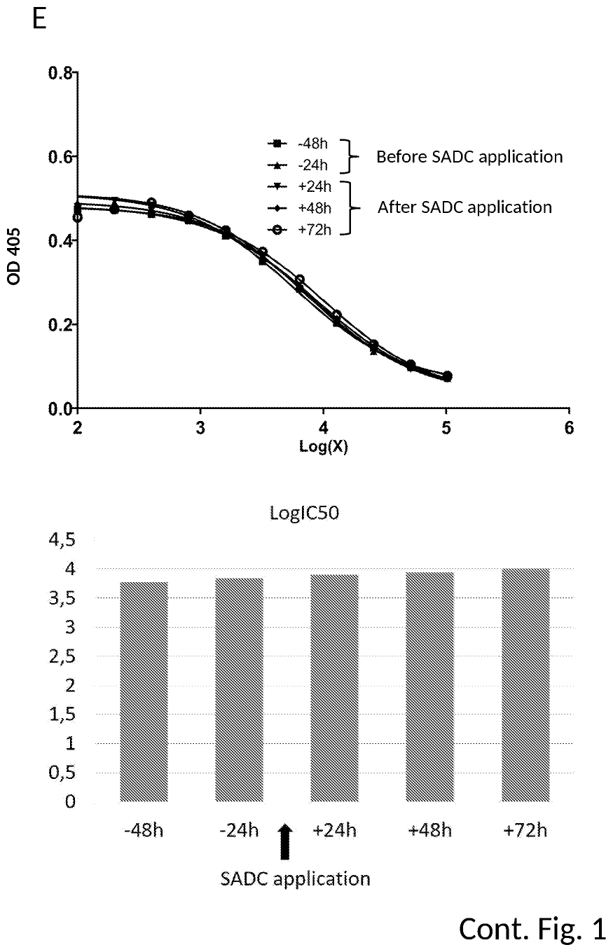

FIG. 1: SADCs successfully reduce the titre of undesired antibodies. Each SADC was applied at time point 0 by i.p. injection into Balb/c mice pre-immunized by peptide immunization against a defined antigen. Each top panel shows anti-peptide titers (0.5× dilution steps; X-axis shows log(X) dilutions) against OD values (y-axis) according to a standard ELISA detecting the corresponding antibody. Each bottom panel shows titers LogIC50 (y-axis) before injection of each SADC (i.e. titers at −48 h and −24 h) and after application of each SADC (i.e. titers +24 h, +48 h and +72 h after injection; indicated on the x-axis). (A) Compound with albumin as the biopolymer scaffold that binds to antibodies directed against EBNA1 (associated with pre-eclampsia). The mice were pre-immunized with a peptide vaccine carrying the EBNA-1 model epitope. (B) Compound with albumin as the biopolymer scaffold that binds to antibodies directed against a peptide derived from the human AChR protein MIR (associated with MG). The mice were pre-immunized with a peptide vaccine carrying the AChR MIR model epitope. (C) Compound with immunoglobulin as the biopolymer scaffold that binds to antibodies directed against EBNA1 (associated with pre-eclampsia). The mice were pre-immunized with a peptide vaccine carrying the EBNA-1 model epitope. (D) Compound with haptoglobin as the biopolymer scaffold that binds to antibodies directed against EBNA1 (associated with pre-eclampsia). The mice were pre-immunized with a peptide vaccine carrying the EBNA-1 model epitope. (E) Demonstration of selectivity using the same immunoglobulin-based SADC binding to antibodies directed against EBNA1 that was used in the experiment shown in panel C. The mice were pre-immunized with an unrelated amino acid sequence. No titre reduction occurred, demonstrating selectivity of the compound.

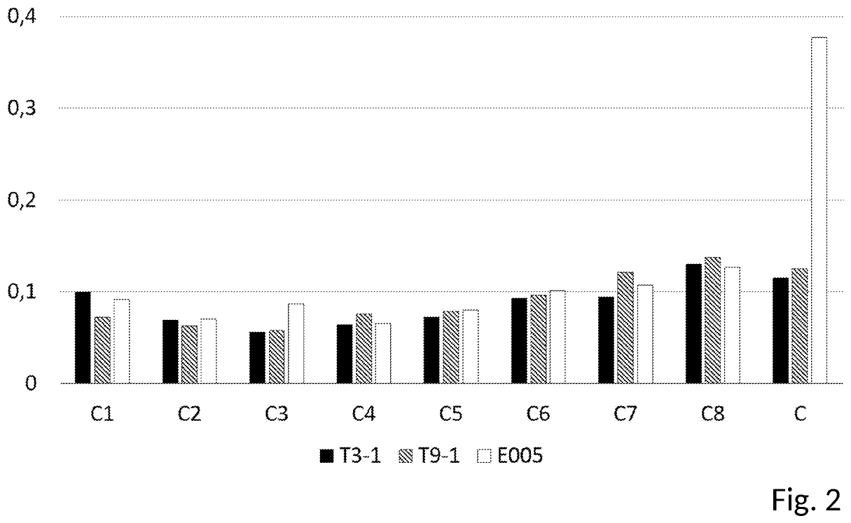

FIG. 2: SADCs are non-immunogenic and do not induce antibody formation after repeated injection into mice. Animals C1-C4 as well as animals C5-C8 were treated i.p. with two different SADCs. Control animal C was vaccinated with a KLH-peptide derived from the human AChR protein MIR. Using BSA-conjugated peptide probes T3-1, T9-1 and E005 (grey bars, as indicated in the graph), respectively, for antibody titer detection by standard ELISA at a dilution of 1:100, it could be demonstrated that antibody induction was absent in animals treated with an SADC, when compared to the vaccine-treated control animal C (y-axis, OD450 nm).

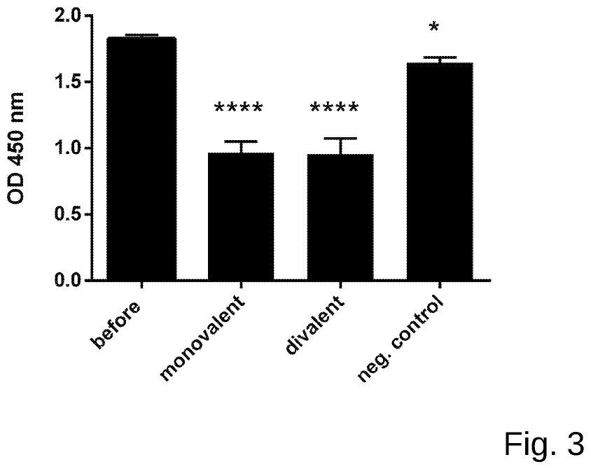

FIG. 3: Successful in vitro depletion of antibodies using SADCs carrying multiple copies of monovalent or divalent peptides. SADCs with mono- or divalent peptides were very suitable to adsorb antibodies and thereby deplete them. “Monovalent” means that peptide monomers are bound to the biopolymer scaffold (i.e. n=1) whereas “divalent” means that peptide dimers are bound to the biopolymer scaffold (i.e. n=2). In the present case, the divalent peptides were “homodivalent”, i.e. the peptide n-mer of the SADC is E006-spacer-E006).

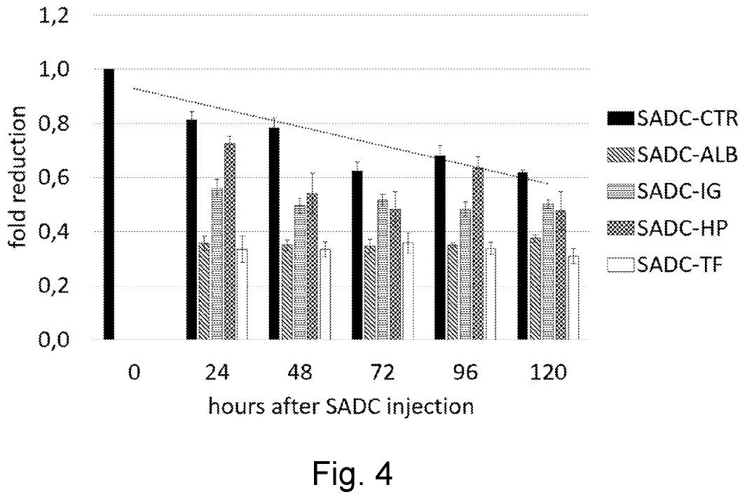

FIG. 4: Rapid, selective antibody depletion in mice using various SADC biopolymer scaffolds. Treated groups exhibited rapid and pronounced antibody reduction already at 24 hrs (in particular SADC-TF) when compared to the mock treated control group SADC-CTL (containing an unrelated peptide). SADC with albumin scaffold—SADC-ALB, SADC with immunoglobulin scaffold—SADC-IG, SADC with haptoglobin scaffold—SADC-HP, and SADC with transferrin scaffold—SADC-TF.

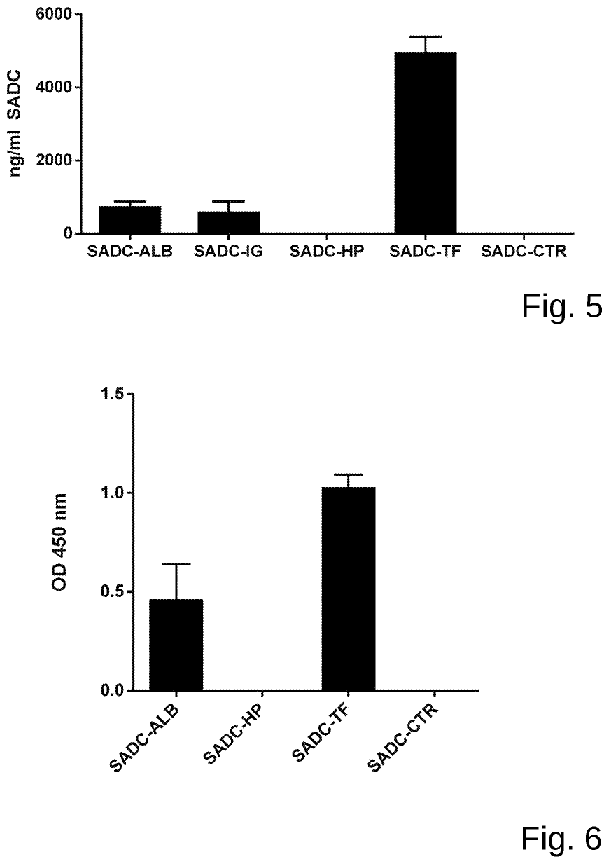

FIG. 5: Detection of SADCs in plasma via their peptide moieties 24 hrs after SADC injection. Both haptoglobin-scaffold-based SADCs (SADC-HP and SADC-CTL) exhibited a relatively shorter plasma half life which represents an advantage over SADCs with other biopolymer scaffolds such as SADC-ALB, SADC-IG oder SADC-TF. SADC with albumin scaffold—SADC-ALB, SADC with immunoglobulin scaffold—SADC-IG, SADC with haptoglobin scaffold—SADC-HP, and SADC with transferrin scaffold—SADC-TF.

FIG. 6: Detection of SADC-IgG complexes in plasma 24 hrs after SADC injection. Haptoglobin based SADCs were subject to accelerated clearance when compared to SADCs with other biopolymer scaffolds. SADC with albumin scaffold—SADC-ALB, SADC with immunoglobulin scaffold—SADC-IG, SADC with haptoglobin scaffold—SADC-HP, and SADC with transferrin scaffold—SADC-TF.

FIG. 7: In vitro analysis of SADC-IgG complex formation. Animals SADC-TF and -ALB showed pronounced immunocomplex formation and binding to C1q as reflected by the strong signals and by sharp signal lowering in case 1000 ng/ml SADC-TF due to the transition from antigen-antibody equilibrium to antigen excess. In contrast, in vitro immunocomplex formation with SADC-HP or SADC-IG were much less efficient when measured in the present assay. These findings corroborate the finding that haptoglobin scaffolds are advantageous over other SADC biopolymer scaffolds because of the reduced propensity to activate the complement system. SADC with albumin scaffold—SADC-ALB, SADC with immunoglobulin scaffold—SADC-IG, SADC with haptoglobin scaffold—SADC-HP, and SADC with transferrin scaffold—SADC-TF.

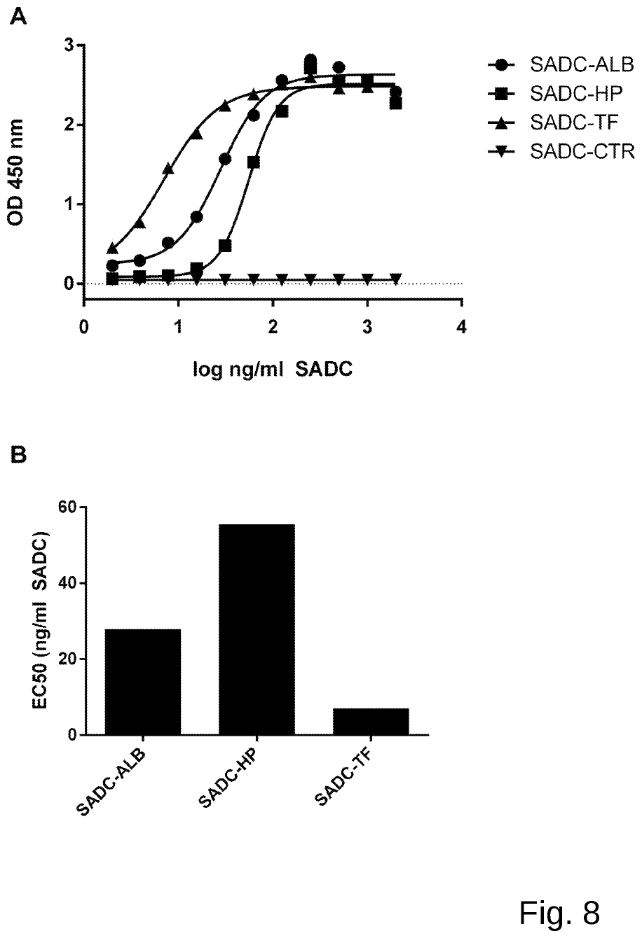

FIG. 8: Determination of IgG capturing by SADCs in vitro. SADC-HP showed markedly less antibody binding capacity in vitro when compared to SADC-TF or SADC-ALB. SADC with albumin scaffold—SADC-ALB, SADC with immunoglobulin scaffold—SADC-IG, SADC with haptoglobin scaffold—SADC-HP, and SADC with transferrin scaffold—SADC-TF.

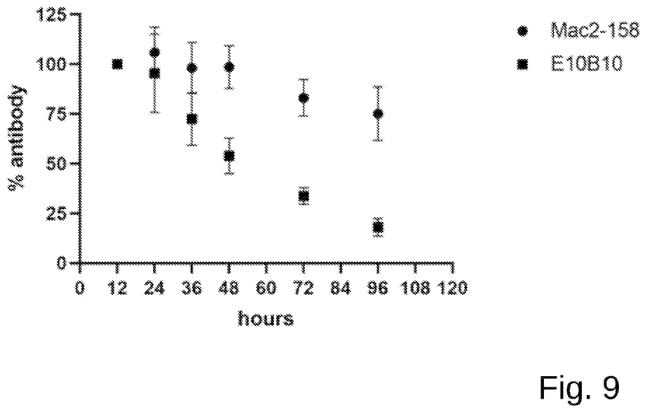

FIG. 9: Blood clearance of an anti-CD163-antibody-based biopolymer scaffold. In a mouse model, mAb E10B10 (specific for murine CD163) is much more rapidly cleared from circulation than mAb Mac2-158 (specific for human CD163 but not for murine CD163, thus serving as negative control in this experiment).

### EXAMPLES

Examples 1-3, 5-8 and 11-13 demonstrate that SADCs are very well suited for selective removal of undesirable antibodies. Examples 4 and 10 as well as 14 and 15 contain more details on the inventive compounds with respect to MG-associated autoantibodies.

### Example 1: SADCs Effectively Reduce the Titre of Undesired Antibodies

Animal models: In order to provide in vivo models with measurable titers of prototypic undesired antibodies in human indications, BALB/c mice were immunized using standard experimental vaccination with KLH-conjugated peptide vaccines derived from established human autoantigens or anti-drug antibodies. After titer evaluation by standard peptide ELISA, immunized animals were treated with the corresponding test SADCs to demonstrate selective antibody lowering by SADC treatment. All experiments were performed in compliance with the guidelines by the corresponding animal ethics authorities.

Immunization of mice with model antigens: Female BALB/c mice (aged 8-10 weeks) were supplied by Janvier (France), maintained under a 12 h light/12 h dark cycle and given free access to food and water. Immunizations were performed by s.c. application of KLH carrier-conjugated peptide vaccines injected 3 times in biweekly intervals. KLH conjugates were generated with peptide T3-2 (CGRPQKRPSCIGCKG), which represents an example for molecular mimicry between a viral antigen (EBNA-1) and an endogenous human receptor antigen, namely the placental GPR50 protein, that was shown to be relevant to preeclampsia (Elliott et al.). In order to confirm the generality of this approach, a larger antigenic peptide derived from the autoimmune condition MG was used for immunization of mice with a human autoepitope. In analogy to peptide T3-2, animals were immunized with peptide T1-1 (LKWNPDDYGGVKKIHIPSEKGC), derived from the MIR (main immunogenic region) of the human AChR protein which plays a fundamental role in pathogenesis of the disease (Luo et al., 2009). The T1-1 peptide was used for immunizing mice with a surrogate partial model epitope of the human AChR autoantigen. The peptide T8-1 (DHTLYTPYHTHPG) was used to immunize control mice to provide a control titer for proof of selectivity of the system. For vaccine conjugate preparation, KLH carrier (Sigma) was activated with sulfo-GMBS (Cat. Nr. 22324 Thermo), according to the manufacturer's instructions, followed by addition of either N- or C-terminally cysteinylated peptides T3-2 and T1-1 and final addition of Alhydrogel© before injection into the flank of the animals. The doses for vaccines T3-2 and T1-1 were 15 μg of conjugate in a volume of 100 ul per injection containing Alhydrogel® (InvivoGen VAC-Alu-250) at a final concentration of 1% per dose.

Generation of prototypic SADCs: For testing selective antibody lowering activity by SADCs of T3-2 and T1-1 immunized mice, SADCs were prepared with mouse serum albumin (MSA) or mouse immunoglobulin (mouse-Ig) as biopolymer scaffold in order to provide an autologous biopolymer scaffold, that will not induce any immune reaction in mice, or non-autologous human haptoglobin as biopolymer scaffold (that did not induce an allogenic reaction after one-time injection within 72 hours). N-terminally cysteinylated SADC peptide E049 (GRPQKRPSCIG) and/or C-terminally cysteinylated SADC peptide E006 (VKKIHIPSEKG) were linked to the scaffold using sulfo-GMBS (Cat. Nr. 22324 Thermo)-activated MSA (Sigma; Cat. Nr. A3559) or -mouse-Ig (Sigma, I5381) or -human haptoglobin (Sigma H0138) according to the instructions of the manufacturer, thereby providing MSA-, Ig- and haptoglobin-based SADCs with the corresponding cysteinylated peptides, that were covalently attached to the lysines of the corresponding biopolymer scaffold. Beside conjugation of the cysteinylated peptides to the lysines via a bifunctional amine-to-sulfhydryl crosslinker, a portion of the added cysteinylated SADC peptides directly reacted with sulfhydryl groups of cysteines of the albumin scaffold protein, which can be detected by treating the conjugates with DTT followed by subsequent detection of free peptides using mass spectrometry or any other analytical method that detects free peptide. Finally, these SADC conjugates were dialysed against water using Pur-A-Lyzer™ (Sigma) and subsequently lyophilized. The lyophilized material was resuspended in PBS before injection into animals.

In vivo functional testing of SADCs: Prototypic SADCs, SADC-E049 and SADC-E006 were injected intraperitoneally (i.p.; as a surrogate for an intended intravenous application in humans and larger animals) into the mice that had previously been immunized with peptide vaccine T3-2 (carrying the EBNA-1 model epitope) and peptide vaccine T1-1 (carrying the AChR MIR model epitope). The applied dose was 30 μg SADC conjugate in a volume of 50 μl PBS. Blood takes were performed by submandibular vein puncture, before (−48 h, −24 h) and after (+24 h, +48 h, +72 h, etc.) i.p. SADC injections, respectively, using capillary micro-hematocrit tubes. Using ELISA analysis (see below), it was found that both prototypic SADCs were able to clearly reduce the titers over a period of at least 72 hrs in the present animal model. It could therefore be concluded that SADCs can be used to effectively reduce titers in vivo.

Titer analysis: Peptide ELISAs were performed according to standard procedures using 96-well plates (Nunc Medisorp plates; Thermofisher, Cat Nr 467320) coated for 1 h at RT with BSA-coupled peptides (30 nM, dissolved in PBS) and incubated with the appropriate buffers while shaking (blocking buffer, 1% BSA, lx PBS; washing buffer, 1×PBS/0.1% Tween; dilution buffer, 1×PBS/0.1% BSA/0.1% Tween). After serum incubation (dilutions starting at 1:50 in PBS; typically in 1:3 or 1:2 titration steps), bound antibodies were detected using Horseradish Peroxidase-conjugated goat anti-mouse IgG (Fc) from Jackson immunoresearch (115-035-008). After stopping the reaction, plates were measured at 450 nm for 20 min using TMB. EC50 were calculated from readout values using curve fitting with a 4-parameter logistic regression model (GraphPad Prism) according to the procedures recommended by the manufacturer. Constraining parameters for ceiling and floor values were set accordingly, providing curve fitting quality levels of R²>0.98.

FIG. 1A shows an in vivo proof of concept in a mouse model for in vivo selective plasma-lowering activity of a prototypic albumin-based SADC candidate that binds to antibodies directed against EBNA1, as a model for autoantibodies and mimicry in preeclampsia (Elliott et al.). For these mouse experiments, mouse albumin was used, in order to avoid any reactivity against a protein from a foreign species. Antibody titers were induced in 6 months old Balb/c mice by standard peptide vaccination. The bottom panel demonstrates that titers LogIC50 (y-axis) before SADC injection (i.e. titers at −48 h and −24 h) were higher than titers LogIC50 after SADC application (i.e. titers +24 h, +48 h and +72 h after injection; indicated on the x-axis).

A similar example is shown in FIG. 1B, using an alternative example of a peptidic antibody binding moiety for a different disease indication. Antibody lowering activity of an albumin-based SADC in a mouse model that was pre-immunized with a different peptide derived from the human AChR protein MIR region (Luo et al., 2009) in order to mimic the situation in MG. The induced antibody titers against the AChR-MIR region were used as surrogate for anti-AChR-MIR autoantibodies known to play a causative role in MG (reviewed by Vincent et al.). A clear titer reduction was seen after SADC application.

FIGS. 1C and 1D demonstrate the functionality of SADC variants comprising alternative biopolymer scaffolds. Specifically, FIG. 1C shows that an immunoglobulin scaffold can be successfully used whereas FIG. 1D demonstrates the use of a haptoglobin-scaffold for constructing an SADC. Both examples show an in vivo proof of concept for selective antibody lowering by an SADC, carrying covalently bound example peptide E049.

The haptoglobin-based SADC was generated using human Haptoglobin as a surrogate although the autologous scaffold protein would be preferred. In order to avoid formation of anti-human-haptoglobin antibodies, only one single SADC injection per mouse of the non-autologous scaffold haptoglobin was used for the present experimental conditions. As expected, under the present experimental conditions (i.e. one-time application), no antibody reactivity was observed against the present surrogate haptoglobin homologue.

FIG. 1E demonstrates the selectivity of the SADC system. The immunoglobulin-based SADC carrying the peptide E049 (i.e. the same as in FIG. 1C) cannot reduce the Ig-titer that was induced by a peptide vaccine with an unrelated, irrelevant amino acid sequence, designated peptide T8-1 (DHTLYTPYHTHPG). The example shows an in vivo proof of concept for the selectivity of the system. The top panel shows anti-peptide T8-1 titers (0.5× dilution steps starting from 1:50 to 1:102400; X-axis shows log(X) dilutions) against OD values (y-axis) according to a standard ELISA. T8-1-titers are unaffected by administration of SADC-Ig-E049 after application. The bottom panel demonstrates that the initial titers LogIC50 (y-axis) before SADC injection (i.e. titers at −48 h and −24 h) are unaffected by administration of SADC-Ig-E049 (arrow) when compared to the titers LogIC50 after SADC application (i.e. titers +24 h, +48 h and +72 h; as indicated on the x-axis), thereby demonstrating the selectivity of the system.

### Example 2: Immunogenicity of SADCs

In order to exclude immunogenicity of SADCs, prototypic candidate SADCs were tested for their propensity to induce antibodies upon repeated injection. Peptides T3-1 and T9-1 were used for this test. T3-1 is a 10-amino acid peptide derived from a reference epitope of the Angiotensin receptor, against which agonistic autoantibodies are formed in a pre-eclampsia animal model (Zhou et al.); T9-1 is a 12-amino acid peptide derived from a reference anti-drug antibody epitope of human IFN gamma (Lin et al.). These control SADC conjugates were injected 8× every two weeks i.p. into naïve, non-immunized female BALB/c mice starting at an age of 8-10 weeks.

Animals C1-C4 were treated i.p. (as described in example 1) with SADC T3-1. Animals C5-C8 were treated i.p. with an SADC carrying the peptide T9-1. As a reference signal for ELISA analysis, plasma from a control animal that was vaccinated 3 times with KLH-peptide T1-1 (derived from the AChR-MIR, explained in Example 1) was used. Using BSA-conjugated peptide probes T3-1, T9-1 and E005 (GGVKKIHIPSEK), respectively, for antibody titer detection by standard ELISA at a dilution of 1:100, it could be demonstrated that antibody induction was absent in SADC-treated animals, when compared to the vaccine-treated control animal C (see FIG. 2). The plasmas were obtained by submandibular blood collection, 1 week after the 3rd vaccine injection (control animal C) and after the last of 8 consecutive SADC injections in 2-weeks intervals (animals C1-C8), respectively. Thus it was demonstrated that SADCs are non-immunogenic and do not induce antibody formation after repeated injection into mice.

### Example 3: Successful In Vitro Depletion of Antibodies Using SADCs Carrying Multiple Copies of Monovalent or Divalent Peptides

Plasma of E006-KLH (VKKIHIPSEKG with C-terminal cysteine, conjugated to KLH) vaccinated mice was diluted 1:3200 in dilution buffer (PBS+0.1% w/v BSA+0.1% Tween20) and incubated (100 μl, room temperature) sequentially (10 min/well) four times on single wells of a microtiter plate that was coated with 2.5 μg/ml (250 ng/well) of SADC or 5 μg/ml (500 ng/well) albumin as negative control.

In order to determine the amount of free, unbound antibody present before and after incubation on SADC coated wells, 50 μl of the diluted serum were taken before and after the depletion and quantified by standard ELISA using E006-BSA coated plates (10 nM peptide) and detection by goat anti mouse IgG bio (Southern Biotech, diluted 1:2000). Subsequently, the biotinylated antibody was detected with Streptavidin-HRP (Thermo Scientific, diluted 1:5000) using TMB as substrate. Development of the signal was stopped with 0.5 M sulfuric acid.

ELISA was measured at OD450 nm (y-axis). As a result, the antibody was efficiently adsorbed by either coated mono- or divalent SADCs containing peptide E006 with C-terminal cysteine (sequence VKKIHIPSEKGC) (before=non-depleted starting material; mono-divalent corresponds to peptides displayed on the SADC surface; neg. control was albumin; indicated on the x-axis). See FIG. 3. (“Monovalent” means that peptide monomers are bound to the biopolymer scaffold (i.e. n=1) whereas “divalent” means that peptide dimers are bound to the biopolymer scaffold (i.e. n=2). In the present case, the divalent peptides were “homodivalent”, i.e. the peptide n-mer of the SADC is E006-S-E006.) This demonstrates that SADCs with mono- or divalent peptides are very suitable to adsorb antibodies and thereby deplete them.

### Example 4: Generation of Mimotope-Based SADCs

Linear and circular peptides derived from wild-type or modified peptide amino acid sequences can be used for the construction of specific SADCs for the selective removal of MG-associated antibodies. In case of a particular epitope, linear peptides or constrained peptides such as cyclopeptides containing portions of an epitope or variants thereof, where for example, one or several amino acids have been substituted or chemically modified in order to improve affinity to an antibody (mimotopes), can be used for constructing SADCs. A peptide screen can be performed with the aim of identifying peptides with optimized affinity to a disease-inducing autoantibody. The flexibility of structural or chemical peptide modification provided a solution to minimize the risk of immunogenicity, in particular of binding of the peptide to HLA and thus the risk of unwanted immune stimulation.

Therefore, wild-type as well as modified linear and circular peptide sequences are derived from AChR MIR. Peptides of various length and positions are systematically permutated by amino acid substitutions and synthesized on a peptide array. This allows screening of 60000 circular and linear wild-type and mimotope peptides derived from these sequences. The peptide arrays are incubated with myasthenogenic autoantibody. This autoantibody is therefore used to screen the 60000 peptides and 100 circular and 100 linear peptide hits are selected based on their relative binding strength to the autoantibody. Of these 200 peptides, 51 sequences are identical between the circular and the linear peptide group. All of the best peptides identified have at least one amino acid substitution when aligned to the original sequences, respectively and are therefore regarded as mimotopes. Also, higher binding strengths can be achieved with circularized peptides.

These newly identified peptides, preferentially those with high relative binding values, are used to generate SADCs for depleting MG-associated autoantibodies.

### Example 5: Rapid, Selective Antibody Depletion in Mice Using Various SADC Biopolymer Scaffolds

10 μg of model undesired antibody mAb anti V5 (Thermo Scientific) was injected i.p. into female Balb/c mice (5 animals per treatment group; aged 9-11 weeks) followed by intravenous injection of 50 μg SADC (different biopolymer scaffolds with tagged V5 peptides bound, see below) 48 hrs after the initial antibody administration. Blood was collected at 24 hrs intervals from the submandibular vein. Blood samples for time point 0 hrs were taken just before SADC administration.

Blood was collected every 24 hrs until time point 120 hrs after the SADC administration (x-axis). The decay and reduction of plasma anti-V5 IgG levels after SADC administration was determined by anti V5 titer readout using standard ELISA procedures in combination with coated V5-peptide-BSA (peptide sequence IPNPLLGLDC) and detection by goat anti mouse IgG bio (Southern Biotech, diluted 1:2000) as shown in FIG. 4. In addition, SADC levels (see Example 6) and immunocomplex formation (see Example 7) were analyzed.

EC50[OD450] values were determined using 4 parameter logistic curve fitting and relative signal decay between the initial level (set to 1 at time point 0) and the following time points (x-axis) was calculated as ratio of the EC50 values (y-axis, fold signal reduction EC50). All SADC peptides contained tags for direct detection of SADC and immunocomplexes from plasma samples; peptide sequences used for SADCs were: IPNPLLGLDGGSGDYKDDDDKGK-(BiotinAca)GC (SADC with albumin scaffold—SADC-ALB, SADC with immunoglobulin scaffold—SADC-IG, SADC with haptoglobin scaffold—SADC-HP, and SADC with transferrin scaffold—SADC-TF) and unrelated peptide VKKIHIPSEKGGSGDYKDDDDKGK-(BiotinAca)GC as negative control SADC (SADC-CTR).

The SADC scaffolds for the different treatment groups of 5 animals are displayed in black/grey shades (see inset of FIG. 4).

Treated groups exhibited rapid and pronounced antibody reduction already at 24 hrs (in particular SADC-TF) when compared to the mock treated control group SADC-CTL. SADC-CTR was used as reference for a normal antibody decay since it has no antibody lowering activity because its peptide sequence is not recognized by the administered anti V5 antibody. The decay of SADC-CTR is thus marked with a trend line, emphasizing the antibody level differences between treated and mock treated animals.

In order to determine the effectivity of selective antibody lowering under these experimental conditions, a two-way ANOVA test was performed using a Dunnett's multiple comparison test. 48 hrs after SADC administration, the antibody EC50 was highly significantly reduced in all SADC groups (p<0.0001) compared to the SADC-CTR reference group (trend line). At 120 hrs after SADC administration, antibody decrease was highly significant in the SADC-ALB and SADC-TF groups (both p<0.0001) and significant in the SADC-HP group (p=0.0292), whereas the SADC-IG group showed a trend towards an EC50 reduction (p=0.0722) 120 hrs after SADC administration. Of note, selective antibody reduction was highly significant (p<0.0001) in the SADC-ALB and SADC-TF groups at all tested time-points after SADC administration.

It is concluded that all SADC biopolymer scaffolds were able to selectively reduce antibody levels. Titer reduction was most pronounced with SADC-ALB and SADC-TF and no rebound or recycling of antibody levels was detected towards the last time points suggesting that undesired antibodies are degraded as intended.

### Example 6: Detection of SADCs in Plasma 24 Hrs after SADC Injection

Plasma levels of different SADC variants at 24 hrs after i.v. injection into Balb/c mice. Determination of Plasma levels (y-axis) of SADC-ALB, -IG, -HP, -TF and the negative control SADC-CTR (x-axis), were detected in the plasmas from the animals already described in example 5. Injected plasma SADC levels were detected by standard ELISA whereby SADCs were captured via their biotin moieties of their peptides in combination with streptavidin coated plates (Thermo Scientific). Captured SADCs were detected by mouse anti Flag-HRP antibody (Thermo Scientific, 1:2,000 diluted) detecting the Flag-tagged peptides (see also example 7):

Assuming a theoretical amount in the order of 25 μg/ml in blood after injecting 50 μg SADC i.v., the detectable amount of SADC ranged between 799 and 623 ng/ml for SADC-ALB or SADC-IG and up to approximately 5000 ng/ml for SADC-TF, 24 hrs after SADC injection. However surprisingly and in contrast, SADC-HP and control SADC-CTR (which is also a SADC-HP variant, however carrying the in this case unrelated negative control peptide E006, see previous examples), had completely disappeared from circulation 24 hrs after injection, and were not detectable anymore. See FIG. 5.

This demonstrates that both Haptoglobin scaffold-based SADCs tested in the present example ((namely SADC-HP and SADC-CTR) exhibit a relatively shorter plasma half-life which represents an advantage over SADCs such as SADC-ALB, SADC-IG oder SADC-TF in regard of their potential role in complement-dependent vascular and renal damage due to the in vivo risk of immunocomplex formation. Another advantage of SADC-HP is the accelerated clearance rate of their unwanted target antibody from blood in cases where a rapid therapeutic effect is needed. The present results demonstrate that Haptoglobin-based SADC scaffolds (as represented by SADC-HP and SADC-CTR) are subject to rapid clearance from the blood, regardless of whether SADC-binding antibodies are present in the blood, thereby minimizing undesirable immunocomplex formation and showing rapid and efficient clearance. Haptoglobin-based SADCs such as SADC-HP in the present example thus provide a therapeutically relevant advantage over other SADC biopolymer scaffolds, such as demonstrated by SADC-TF or SADC-ALB, both of which are still detectable 24 hrs after injection under the described conditions, in contrast to SADC-HP or SADC-CTR which both are completely cleared 24 hrs after injection.

### Example 7: Detection of SADC-IgG Complexes in Plasma 24 Hrs after SADC Injection

In order to determine the amount IgG bound to SADCs in vivo, after i.v. injection of 10 μg anti V5 IgG (Thermo Scientific) followed by injection of SADC-ALB, -HP, -TF and -CTR (50 μg) administered i.v. 48 h after antibody injection, plasma was collected from the submandibular vein, 24 hrs after SADC injection, and incubated on streptavidin plates for capturing SADCs from plasma via their biotinylated SADC-V5-peptide [IPNPLLGLDGGSGDYKDDDDKGK(BiotinAca)GC or in case of SADC-CTR the negative control peptide VKKIHIPSEKGGSGDYKDDDDKGK (BiotinAca)GC]. IgG bound to the streptavidin-captured SADCs was detected by ELISA using a goat anti mouse IgG HRP antibody (Jackson Immuno Research, diluted 1:2,000) for detection of the SADC-antibody complexes present in plasma 24 hrs after SADC injection. OD450 nm values (y-axis) obtained for a negative control serum from untreated animals were subtracted from the OD450 nm values of the test groups (x-axis) for background correction.

As shown in FIG. 6, pronounced anti-V5 antibody signals were seen in case of SADC-ALB and SADC-TF injected mice (black bars represent background corrected OD values at a dilution of 1:25, mean value of 5 mice; standard deviation error bars), whereas no antibody signal could be detected in plasmas from SADC-HP or control SADC-CTR injected animals (SADC-CTR is a negative control carrying the irrelevant peptide bio-FLG-E006 [VKKIHIPSEKGGSGDYKDDDDKGK(BiotinAca)GC] that is not recognized by any anti V5 antibody). This demonstrates the absence of detectable amounts of SADC-HP/IgG complexes in the plasma 24 hrs after i.v. SADC application.

SADC-HP is therefore subject to accelerated clearance in anti V5 pre-injected mice when compared to SADC-ALB or SADC-TF.

### Example 8: In Vitro Analysis of SADC-Immunoglobulin Complex Formation

SADC-antibody complex formation was analyzed by pre-incubating 1 μg/ml of human anti V5 antibody (anti V5 epitope tag [SV5-P-K], human IgG3, Absolute Antibody) with increasing concentrations of SADC-ALB, -IG, -HP, -TF and -CTR (displayed on the x-axis) in PBS+0.1% w/v BSA+0.1% v/v Tween20 for 2 hours at room temperature in order to allow for immunocomplex formation in vitro. After complex formation, samples were incubated on ELISA plates that had previously been coated with 10 μg/ml of human C1q (CompTech) for 1 h at room temperature, in order to allow capturing of in vitro formed immunocomplexes. Complexes were subsequently detected by ELISA using anti human IgG (Fab specific)-Peroxidase (Sigma, diluted 1:1,000). Measured signals at OD450 nm (y-axis) reflect Antibody-SADC complex formation in vitro.

As shown in FIG. 7, SADC-TF and -ALB showed pronounced immunocomplex formation and binding to C1q as reflected by the strong signals and by sharp signal lowering in case 1000 ng/ml SADC-TF due to the transition from antigen-antibody equilibrium to antigen excess. In contrast, in vitro immunocomplex formation with SADC-HP or SADC-IG were much less efficient when measured in the present assay.

Together with the in vivo data (previous examples), these findings corroborate the finding that haptoglobin scaffolds are advantageous over other SADC biopolymer scaffolds because of the reduced propensity to activate the complement system. In contrast, SADC-TF or SADC-ALB show higher complexation, and thereby carry a certain risk of activating the C1 complex with initiation of the classical complement pathway (a risk which may be tolerable in some settings, however).

### Example 9: Determination of IgG Capturing by SADCs In Vitro

Immunocomplexes were allowed to form in vitro, similar to the previous example, using 1 μg/ml mouse anti V5 antibody (Thermo Scientific) in combination with increasing amounts of SADCs (displayed on the x-axis). SADC-antibody complexes were captured on a streptavidin coated ELISA plate via the biotinylated SADC-peptides (see previous examples), followed by detection of bound anti-V5 using anti mouse IgG-HRP (Jackson Immuno Research, diluted 1:2,000).

Under these assay conditions, SADC-HP showed markedly less antibody binding capacity in vitro when compared to SADC-TF or SADC-ALB (see FIG. 8, A). The calculated EC50 values for IgG detection on SADCs were 7.0 ng/ml, 27.9 ng/ml and 55.5 ng/ml for SADC-TF, -ALB and -HP, respectively (see FIG. 8, B).

This in vitro finding is consistent with the observation (see previous examples) that SADC-HP has a lower immunocomplex formation capacity when compared to SADC-TF or SADC-ALB which is regarded as a safety advantage with respect to its therapeutic use for the depletion of unwanted antibodies.

### Example 10: SADCs to Reduce Undesired MG-Associated Autoantibodies

Three SADCs are provided to reduce MG-associated autoantibodies:
(a) SADC-a with Mac2-158 (as disclosed in WO 2011/039510 A2) as biopolymer scaffold and at least two peptides with the sequence LRRNPAD covalently bound to the scaffold,
(b) SADC-b with human transferrin as biopolymer scaffold and at least two peptides with the cyclic sequence VRLRWNPADYP covalently bound to the scaffold, and
(c) SADC-c with human albumin as biopolymer scaffold and at least two peptides with the sequence YNLKWNPDDY covalently bound to the scaffold.

These SADCs are administered to an individual with MG.

### Example 11: In-Vivo Function of Anti-CD163-Antibody-Based SADC Biopolymer Scaffold

Rapid in vivo blood clearance of anti-mouse-CD163 mAb E10B10 (as disclosed in WO 2011/039510 A2). mAb E10B10 was resynthesized with a mouse IgG2a backbone. 50 μg mAb E10B10 and Mac2-158 (human-specific anti-CD163 mAb as disclosed in WO 2011/039510 A2, used as negative control in this example since it does not bind to mouse CD163) were injected i.v. into mice and measured after 12, 24, 36, 48, 72, 96 hours in an ELISA to determine the blood clearance.

mAb E10B10 was much more rapidly cleared from circulation than control mAb Mac2-158 was, as shown in FIG. 9, since E10B10 binds to the mouse CD163 whereas Mac2-158 is human-specific, although both were expressed as mouse IgG2a isotypes for direct comparison.

In conclusion, anti-CD163 antibodies are highly suitable as SADC scaffold because of their clearance profile. SADCs with such scaffolds will rapidly clear undesirable antibodies from circulation.

Detailed methods: 50 ug of biotinylated monoclonal antibodies E10B10 and biotinylated Mac2-158 were injected i.v. into mice and measured after 12, 24, 36, 48, 72, 96 hours to determine the clearance by ELISA: Streptavidin plates were incubated with plasma samples diluted in PBS+0.1% BSA+0.1% Tween20 for 1 h at room temperature (50 μl/well). After washing (3×with PBS+0.1% Tween20), bound biotinylated antibodies were detected with anti-mouse IgG+IgM-HRP antibody at a 1:1000 dilution. After washing, TMB substrate was added and development of the substrate was stopped with TMB Stop Solution. The signal at OD450 nm was read. The EC50 values were calculated by non-linear regression using 4 parametric curve fitting with constrained curves and least squares regression. EC50 values at time-point T12 (this was the first measured time-point after antibody injection) was set at 100%, all other EC50 values were compared to the levels at T12.

### Example 12: Epitope Mapping of Anti-CD163 mAbs

mAb E10B10 provides CD163-mediated, accelerated in vivo clearance from blood in mice (see example 11). The epitope of this antibody was fine mapped using circular peptide arrays, whereby the peptides were derived from mouse CD163. As a result, a peptide cluster that is recognized by mAb E10B10 was identified (see example 13).

The same epitope mapping procedure using circularized peptides was performed with mAb Mac2-158 (as disclosed in WO 2011/039510 A2). Epitope mapping results for mAb Mac2-158 yielded two peptide clusters (see example 13) which allowed further demarcation of CD163 epitope regions that are especially relevant to internalization of ligands and antibodies that bind to the receptor.

These newly characterized epitopes for Mac2-158 and E10B10 thus revealed three preferred binding regions for antibodies against CD163. Based on the fine epitope mapping work, linear or preferentially circular peptides are synthesized and used for the induction, production and selection of polyclonal or monoclonal antibodies or other CD163-binding SADC scaffolds that target CD163.

### Example 13: Epitope Mapping of Anti-CD163 mAbs

Peptides aligned to SRCR domain 1 of human CD163 were selected from the top 20 peptide hits of mAb Mac2-158 circular epitope mapping peptides and the most preferred sequences were selected from two peptide alignment clusters at the N-terminus and at the C-terminus of SRCR-1 of human CD163. As a result, the following sequences (as well as motifs derived therefrom) are highly suitable epitopes anti-CD163 antibodies and fragments thereof used as SADC biopolymer scaffold:

### Peptide Cluster 1:

| | |
|---|---|
| 04 | -----------------EWGTVCNNGWSME------ |
| 07 | -----CSGRVEVKVQEEW------------------ |
| 09 | --------------QEEWGTVCNNGWS--------- |
| 12 | -----------------WGTVCNNGWSMEA------ |
| 14 | ---------------EEWGTVCNNGWSM-------- |
| 18 | -------------VQEEWGTVCNNGW---------- |
| 19 | ----------------EWGTVCNNGW---------- |
| 20 | -----------------WGTVCNNGWS--------- |
| huCD163-domain | DGENKCSGRVEVKVQEEWGTVCNNGWSMEAVSVICN |
| 1-3 | |

### Peptide Cluster 2:

| | |
|---|---|
| 01 | ------------ESALWDC-------------- |
| 02 | ---------RGNESALWDC-------------- |
| 03 | -------SCRGNESALW---------------- |
| 05 | ------VSCRGNESALWDC-------------- |
| 06 | --------------ALWDCKHDGW--------- |
| 08 | ----DHVSCRGNESALW---------------- |
| 11 | --------CRGNESALWD--------------- |
| 13 | -----------NESALWDCKHDGW--------- |
| 17 | ------------ESALWDCKHDGWG-------- |
| huCD163-domain | RIWMDHVSCRGNESALWDCKHDGWGKHSNCTHQ |
| 1-3 | |

Fine epitope mapping of mAb E10B10 was performed as for Mac2-158. 1068 circular peptides (sized 7, 10 and 13 amino acids) and derived from SRCR-1 to -3 of the mouse CD163 sequence (UniProKB Q2VLH6.2) were screened with mAb E10B10 and the following top binding peptides were obtained (ranked by relative signal strength). The human CD163 sequence was aligned to this cluster of mouse CD163 sequences, revealing another highly suitable epitope:

### Peptide Cluster 3:

| | |
|---|---|
| 01 | ---------------------VTNAPGEMKKELR--------- |
| 02 | ------------------ASAVTNAPGEMKK------------ |
| 03 | ---------------------VTNAPGEMKK------------ |
| 04 | ---------------------VTNAPGE--------------- |
| 05 | ----------------GSASAVTNAPGEM-------------- |
| 06 | --------------------AVTNAPGEMKKEL---------- |
| 07 | -----------------SASAVTNAPGEMK------------- |
| 08 | ---------------SGSASAVTNAPGE--------------- |
| 09 | --------------------AVTNAPGEMK------------- |
| 10 | -------------------SAVTNAPGEM-------------- |
| 11 | ------------------ASAVTNAPGE--------------- |
| 12 | -------------------SAVTNAPGEMKKE----------- |
| 13 | ----------------------TNAPGEMKKE----------- |
| mCD163 (SRCR-1, N-terminus) | VTNAPGEMKKELRLAGGENNCS |
| hCD163 (SRCR-1, N-terminus) | SSLGGTDKELRLVDGENKCS |

The human homologues of mouse peptides 01-13 from cluster 3 have the following sequences of the N-terminal portion of the mature human CD163 protein (UniProtKB: Q86VB7):

| Cluster 3 peptides (mouse): | human homologues: |
|---|---|
| 01 | SSLGGTDKELR |
| 06 | SSLGGTDKEL |
| 12, 13 | SSLGGTDKE |
| 02, 03 | SSLGGTDK |
| 07, 09 | SSLGGTD |
| 05, 10 | SSLGGT |
| 04, 08, 11 | SSLGG |
| hCD163 (SRCR-1) | SSLGGTDKELRLVDGENKCS |

These homologue peptides represent further highly suitable epitopes for the anti-CD163 antibody-based biopolymer scaffold.

### Example 14: Screening for Linear and Cyclic Mimotope Peptides that are Bound by Myasthenogenic Antibody mAb198

Mimotope peptides were identified that can be used for the generation of SADCs for the depletion and sequestration of autoantibodies directed against the human acetylcholine receptor alpha chain (UniProtKB P02708) in autoimmune diseases with myasthenogenic autoantibodies such as MG. In addition, such peptides can also be used for diagnostic purpose, for the detection of biomarkers and for the exploration and quantification of human autoantibodies in any autoimmune disease with anti AChR-autoantibodies.

A custom peptide array of 30000 peptides was synthesized, with peptides of 7-19 amino acids in size; the peptides were based on substitution analyses of 176 AChR-MIR peptides with exchanges of each amino acid positions by each of the 20 common amino acids. Peptides were synthesized directly on a microarray, and screened with monoclonal antibody mAB 198 in order to obtain mimotopes with a binding strength which was improved over the human AChR alpha chain MIR-containing sequence FSHLQNEQWVDYNLKWNPDDYGGVKKIHI (Tzartos et al, 1991; Luo et al, 2009) and over a previously published mimotope sequence (ETRLVANLLGGGSLRWNPADYGGIKKIRG), that was derived from Torpedo AchR with the help of antibody mAB 132A (Trinh et al, 2014). In particular, the wild type AChR-MIR sequence is known to structurally mimic to a certain extent the epitope recognized by myasthenogenic, unwanted and disease-causing antibodies from patients. The mimotope peptide library was designed by permutation of each single amino acid position of these two sequences with all natural amino acids in order to provide a maximally diversified collection of peptide variants. All peptides were synthesized in doublets as linear and as cyclic peptides. The aim was to obtain peptides (mimotopes) that mimic the natural epitope of myasthenogenic antibodies.

For the mimotope screen, the antibody mAB 198 (Absolute Antibody Ltd, UK) was used. This is a prototype antibody that was generated in rats using human AChR (Tzartos et al, 1983; Asher et al, 1993). It has previously been shown that mAB 198 can induce myasthenic symptoms in rats 24 h after i.v. injection and it was previously characterized as a prototypic disease-inducing antibody. Additionally, animals injected with this antibody show weight loss within 48 hrs after injection. mAB 198 was also found to compete with autoantibodies against AChR-MIR from MG patients (Mamalaki et al, 1993; Graus et al, 1997). Therefore, mAB198 was used as a surrogate probe to screen for new mimotopes that are capable of mimicking the corresponding AChR-MIR epitope or portions of the epitope.

As a readout, binding of mAB 198 to the 30000 linear and cyclic peptides was measured by fluorescence readout, and peptides were ranked according to their relative fluorescent signal after normalization against the background.

The top-ranked 100 linear and cyclic mimotopes are listed in Table 1 and Table 2 above, respectively.

Amongst the top 100 cyclic and top 100 linear peptides identified, none of the hits showed 100% identity when aligned to the original sequences FSHLQNEQWVDYNLKWNPDDYGGVKKIHI or ETRLVANLLGGGSLRWNPADYGGIKKIRG from which the mimotope library was initially designed by permutation design. This demonstrates that the mimotopes identified in this screen are superior binders to mAB198 when compared to peptides with an original sequence from either human AChR (UniProtKB P02708) or the previously published mimotope sequence by Trinh (Thrinh et al, 2014).

To even further improve the binding strength and specificity, these mimotopes are modified by additional sequence permutation rounds or even by chemically modified amino acids in order to achieve improved antibody depletion and sequestration of unwanted, disease-causing antibodies or improved detection of autoantibodies for diagnostic or biomarker discovery and analysis.

### Example 15: Screening for Cyclic Epitope Peptides that are Bound by Myasthenogenic Antibody mAb637

1215 cyclic peptides derived from the human acetylcholine receptor alpha chain (UniProtKB P02708) with a length of 7, 10 and 13 amino acids and a peptide overlap of 6, 9 and 12 amino acids was synthesized similar to the method performed in Example 14, and used for epitope mapping of mAB 637, a previously published, patient-derived MG antibody (Graus et al, 1997).

As a result of this screening, the following top 6 circular peptides were identified and can preferably be used for the depletion and detection of anti AChR-MIR antibodies in MG and other patients with autoimmune diseases or any other purposes disclosed herein. The top confirmed peptides recognized by mAB 637 were:

| | |
|---|---|
| MIR-c03 | ----------WVDYNLKWNPDDY------- |
| MIR-c05 | -------------YNLKWNPDDY------- |
| MIR-c13 | ---------------KWNPDDY-------- |
| MIR-c06 | LFSHLQNEQWVDY----------------- |
| MIR-c15 | ------NEQWVDY----------------- |
| MIR-c20 | ---HLQNEQWVDY----------------- |
| huAChR-MIR | -FSHLONEQWVDYNLKWNPDDYGGVKKIHI |

These peptides are also listed in Table 3 above.

To even further improve the binding strength and specificity, these peptides are modified by additional sequence permutation rounds or even by chemically modified amino acids in order to achieve improved antibody depletion and sequestration of unwanted, disease-causing antibodies or improved detection of autoantibodies for diagnostic or biomarker discovery and analysis.

### Non-Patent References

Asher, Orna, et al. “Increased gene expression of acetylcholine receptor and myogenic factors in passively transferred experimental autoimmune myasthenia gravis.” The Journal of Immunology 151.11 (1993): 6442-6450.
Carter, John Mark, and Larry Loomis-Price. “B cell epitope mapping using synthetic peptides.” Current protocols in immunology 60.1 (2004): 9-4.
Dijkstra, C. D., et al. “The heterogeneity of mononuclear phagocytes in lymphoid organs: distinct macrophage subpopulations in rat recognized by monoclonal antibodies ED1, ED2 and ED3.” Microenvironments in the Lymphoid System. Springer, Boston, M A, 1985. 409-419.
Elliott, Serra E., et al. “A pre-eclampsia-associated Epstein-Barr virus antibody cross-reacts with placental GPR50.” Clinical Immunology 168 (2016): 64-71.
Erlandsson, Ann, et al. “In vivo clearing of idiotypic antibodies with antiidiotypic antibodies and their derivatives.” Molecular immunology 43.6 (2006): 599-606.
Etzerodt, Anders, et al. “Efficient intracellular drug-targeting of macrophages using stealth liposomes directed to the hemoglobin scavenger receptor CD163.” Journal of controlled release 160.1 (2012): 72-80.
Fabriek, Babs O., et al. “The macrophage scavenger receptor CD163 functions as an innate immune sensor for bacteria.” Blood 113.4 (2009): 887-892.
Garces, Jorge Carlos, et al. “Antibody-mediated rejection: a review.” The Ochsner Journal 17.1 (2017): 46.
Gazarian, Karlen, et al. “Mimotope peptides selected from phage display combinatorial library by serum antibodies of pigs experimentally infected with Taenia solium as leads to developing diagnostic antigens for human neurocysticercosis.” Peptides 38.2 (2012): 381-388.
Gfeller, David, et al. “Current tools for predicting cancer-specific T cell immunity.” Oncoimmunology 5.7 (2016): e1177691.
Granfeldt, Asger, et al. “Targeting dexamethasone to macrophages in a porcine endotoxemic model.” Critical Care Medicine 41.11 (2013): e309-e318.
Graus, Yvo F., et al. “Human anti-nicotinic acetylcholine receptor recombinant Fab fragments isolated from thymus-derived phage display libraries from myasthenia gravis patients reflect predominant specificities in serum and block the action of pathogenic serum antibodies.” The Journal of Immunology 158.4 (1997): 1919-1929.
Graversen, Jonas H., et al. “Targeting the hemoglobin scavenger receptor CD163 in macrophages highly increases the anti-inflammatory potency of dexamethasone.” Molecular Therapy 20.8 (2012): 1550-1558.
Gurda, Brittney L., et al. “Mapping a neutralizing epitope onto the capsid of adeno-associated virus serotype 8.” Journal of virology 86.15 (2012): 7739-7751.
Hansen, Lajla Bruntse, Soren Buus, and Claus Schafer-Nielsen. “Identification and mapping of linear antibody epitopes in human serum albumin using high-density peptide arrays.” PLoS One 8.7 (2013): e68902.
Homma, Masayuki, et al. “A Novel Fusion Protein, AChR-Fc, Ameliorates Myasthenia Gravis by Neutralizing Antiacetylcholine Receptor Antibodies and Suppressing Acetylcholine Receptor-Reactive B Cells.” Neurotherapeutics 14.1 (2017): 191-198.
Howard Jr, James F. “Myasthenia gravis: the role of complement at the neuromuscular junction.” Annals of the New York Academy of Sciences 1412.1 (2018): 113-128.
Howarth, M., & Brune, K. D. (2018). New routes and opportunities for modular construction of particulate vaccines: stick, click and glue. Frontiers in immunology, 9, 1432.
Huang, Kun, Yue-Bei Luo, and Huan Yang. “Autoimmune channelopathies at neuromuscular junction.” Frontiers in neurology 10 (2019): 516.
Jansson, Liselotte, et al. “Immunotherapy With Apitopes Blocks the Immune Response to TSH Receptor in HLA-DR Transgenic Mice.” Endocrinology 159.9 (2018): 3446-3457.
Jensen, Kamilla Kjærgaard, et al. “Improved methods for predicting peptide binding affinity to MHC class II molecules.” Immunology 154.3 (2018): 394-406.
Joubert, Bastien, and Jérôme Honnorat. “Autoimmune channelopathies in paraneoplastic neurological syndromes.” Biochimica et Biophysica Acta (BBA)-Biomembranes 1848.10 (2015): 2665-2676.
Jurtz, Vanessa, et al. “NetMHCpan-4.0: improved peptide-MHC class I interaction predictions integrating eluted ligand and peptide binding affinity data.” The Journal of Immunology 199.9 (2017): 3360-3368.
Kainulainen, Markus H., et al. “High-throughput quantitation of SARS-CoV-2 antibodies in a single-dilution homogeneous assay.” Scientific reports 11.1 (2021): 1-9.
Koşalo\check{g}lu-Yalç, Zeynep, et al. “Predicting T cell recognition of MHC class I restricted neoepitopes.” Oncoimmunology 7.11 (2018): e1492508.
Lazaridis, Konstantinos, et al. “Specific removal of autoantibodies by extracorporeal immunoadsorption ameliorates experimental autoimmune myasthenia gravis.” Journal of neuroimmunology 312 (2017): 24-30.
Leung, Nicki Y H, et al. “Screening and identification of mimotopes of the major shrimp allergen tropomyosin using one-bead-one-compound peptide libraries.” Cellular & molecular immunology 14.3 (2017): 308-318.
Li, Peipei, Li Wang, and Li-jun Di. “Applications of protein fragment complementation assays for analyzing biomolecular interactions and biochemical networks in living cells.” Journal of proteome research 18.8 (2019): 2987-2998.
Lim, Sung In, and Inchan Kwon. “Bioconjugation of therapeutic proteins and enzymes using the expanded set of genetically encoded amino acids.” Critical reviews in biotechnology 36.5 (2016): 803-815.
Lin, Chia-Hao, et al. “Identification of a major epitope by anti-interferon-γ autoantibodies in patients with mycobacterial disease.” Nature medicine 22.9 (2016): 994.
Lorentz, Kristen M., et al. “Engineered binding to erythrocytes induces immunological tolerance to E. coli asparaginase.” Science advances 1.6 (2015): e1500112.
Luo, Jie, et al. “Main immunogenic region structure promotes binding of conformation-dependent myasthenia gravis autoantibodies, nicotinic acetylcholine receptor conformation maturation, and agonist sensitivity.” Journal of Neuroscience 29.44 (2009): 13898-13908.
Luo, Jie, and Jon Lindstrom. “AChR-specific immunosuppressive therapy of myasthenia gravis.” Biochemical pharmacology 97.4 (2015): 609-619.
Madsen, Mette, et al. “Molecular Characterization of the Haptoglobin-Hemoglobin Receptor CD163 ligand binding properties of the scavenger receptor cysteine-rich domain region.” Journal of Biological Chemistry 279.49 (2004): 51561-51567.
Majowicz, Anna, et al. “Seroprevalence of pre-existing NABs against AAV1, 2, 5, 6 and 8 in the South African Hemophilia B patient population.” (2019): 3353-3353.
Mamalaki, Avgi, Nikos Trakas And, and Socrates J. Tzartos. “Bacterial expression of a single-chain Fv fragment which efficiently protects the acetylcholine receptor against antigenic modulation caused by myasthenic antibodies.” European journal of immunology 23.8 (1993): 1839-1845.
Masood et al, “Morvan syndrome”, In: StatPearls [Internet]. Treasure Island (FL): StatPearls Publishing; 2021.
Mazor, Ronit, et al. “Tolerogenic nanoparticles restore the antitumor activity of recombinant immunotoxins by mitigating immunogenicity.” Proceedings of the National Academy of Sciences 115.4 (2018): E733-E742.
Meister, Daniel, S. Maryamdokht Taimoory, and John F. Trant. “Unnatural amino acids improve affinity and modulate immunogenicity: Developing peptides to treat MHC type II autoimmune disorders.” Peptide Science 111.1 (2019): e24058.
Mingozzi, Federico, et al. “Overcoming preexisting humoral immunity to AAV using capsid decoys.” Science translational medicine 5.194 (2013): 194ra92-194ra92.
Mingozzi, Federico, and Katherine A. High. “Overcoming the host immune response to adeno-associated virus gene delivery vectors: the race between clearance, tolerance, neutralization, and escape.” Annual review of virology 4 (2017): 511-534.
Morimoto et. al., Bioconjugate Chemistry 25 (8) (2014): 1479-1491
Moussa, Ehab M., et al. “Immunogenicity of therapeutic protein aggregates.” Journal of pharmaceutical sciences 105.2 (2016): 417-430.
Müller, Manuel M. “Post-translational modifications of protein backbones: unique functions, mechanisms, and challenges.” Biochemistry 57.2 (2017): 177-185.
Nakane, Shunya, et al. “Autoimmune autonomic ganglionopathy: an update on diagnosis and treatment.” Expert review of neurotherapeutics 18.12 (2018): 953-965.
Peters, Bjoern, et al. “A community resource benchmarking predictions of peptide binding to MHC-I molecules.” PLoS computational biology 2.6 (2006): e65.
Pishesha, Novalia, et al. “Engineered erythrocytes covalently linked to antigenic peptides can protect against autoimmune disease.” Proceedings of the National Academy of Sciences (2017): 201701746.
Rey et al., Clinical Immunology 96 (3) (2000): 269-279 Ruff, Robert L., and Robert P. Lisak. “Nature and action of antibodies in myasthenia gravis.” Neurologic clinics 36.2 (2018): 275-291.
Rummler, Silke, et al. “Current techniques for ABO-incompatible living donor liver transplantation.” World journal of transplantation 6.3 (2016): 548.
Runcie, Karie, et al. “Bi-specific and tri-specific antibodies—the next big thing in solid tumor therapeutics.” Molecular Medicine 24.1 (2018): 50.
Ryan, Brent J., Ahuva Nissim, and Paul G. Winyard. “Oxidative post-translational modifications and their involvement in the pathogenesis of autoimmune diseases.” Redox biology 2 (2014): 715-724.
Shanmugam, Arulkumaran, et al. “Identification of PSA peptide mimotopes using phage display peptide library.” Peptides 32.6 (2011): 1097-1102.
Siang Ong, Yong, et al. “Recent advances in synthesis and identification of cyclic peptides for bioapplications.” Current topics in medicinal chemistry 17.20 (2017): 2302-2318.
Skytthe, Maria K., Jonas Heilskov Graversen, and Soren K. Moestrup. “Targeting of CD163+ Macrophages in Inflammatory and Malignant Diseases.” International Journal of Molecular Sciences 21.15 (2020): 5497.
Sorensen, Karen Kristine, et al. “Liver sinusoidal endothelial cells.” Comprehensive Physiology 5.4 (2011): 1751-1774.
Spiess, Christoph, Qianting Zhai, and Paul J. Carter. “Alternative molecular formats and therapeutic applications for bispecific antibodies.” Molecular immunology 67.2 (2015): 95-106.
Taddeo, Adriano, et al. “Selection and depletion of plasma cells based on the specificity of the secreted antibody.” European journal of immunology 45.1 (2015): 317-319.
Teschner, Sven, et al. “ABO-incompatible kidney transplantation using regenerative selective immunoglobulin adsorption.” Journal of clinical apheresis 27.2 (2012): 51-60.
Tetala, Kishore K R, et al. “Selective depletion of neuropathy-related antibodies from human serum by monolithic affinity columns containing ganglioside mimics.” Journal of medicinal chemistry 54.10 (2011): 3500-3505.
Trinh, Vu B., Alex J. Foster, and Robert H. Fairclough. “Design, synthesis, and characterization of a 39 amino acid peptide mimic of the main immunogenic region of the Torpedo acetylcholine receptor.” Molecular immunology 59.1 (2014): 79-90.
Tzartos, Socrates, et al. “Demonstration of a main immunogenic region on acetylcholine receptors from human muscle using monoclonal antibodies to human receptor.” FEBS letters 158.1 (1983): 116-118.
Tzartos, S. J., et al. “The main immunogenic region (MIR) of the nicotinic acetylcholine receptor and the anti-MIR antibodies.” Molecular neurobiology 5.1 (1991): 1-29.
Vincent, Angela, et al. “Serological and experimental studies in different forms of myasthenia gravis.” Annals of the New York Academy of Sciences 1413.1 (2018): 143-153.
Wallukat, Gerd, et al. “Patients with preeclampsia develop agonistic autoantibodies against the angiotensin AT 1 receptor.” The Journal of clinical investigation 103.7 (1999): 945-952.
Yoshikawa, et al. “FK506 prevents induction of rat experimental autoimmune myasthenia gravis.” Journal of autoimmunity 10.1 (1997): 11-16.
Zhou, Cissy C., et al. “Angiotensin receptor agonistic autoantibodies induce pre-eclampsia in pregnant mice.” Nature medicine 14.8 (2008): 855.

1. A compound comprising:
a biopolymer scaffold; and at least
a first peptide n-mer of the general formula:
P(—S—P)₍ₙ₋₁₎ and
a second peptide n-mer of the general formula:
P(—S—P)₍ₙ₋₁₎—;
wherein, independently for each occurrence, P is a peptide with a sequence length of 6-13 amino acids, and S is a non-peptide spacer;
wherein, independently for each of the peptide n-mers, n is an integer of at least 1;
wherein each of the peptide n-mers is bound to the biopolymer scaffold, preferably via a linker each;
wherein, independently for each occurrence, P comprises at least 6 consecutive amino acids of a human nicotinic AChR MIR-derived amino-acid sequence selected from SEQ ID NOs: 1-100, SEQ ID NOs: 101-200 and SEQ ID NOs: 201-206; and
wherein at most two amino acids of said amino-acid sequence is independently substituted by any other amino acid.

2. The compound of claim 1, wherein said amino-acid sequence is selected from LRRNPAD, NPADYRG, NPADYHG, VRLRWNPADYP, LRGNPAD, WNPADYR, LRFNPAD, GSLRYNP, LRVNPADYG, LRRNPADYG, VRLRWNPADYP, RLRVNPADY, LRVNPADYG, WIDVRLRGNPA, RLNPADY, RFNPADY, RLRLNPADY, RLRGNPADY, DVRLRINPADY, DVRLRVNPADY, WVDYNLKWNPDDY, YNLKWNPDDY, KWNPDDY, LFSHLQNEQWVDY, NEQWVDY and HLQNEQWVDY.

3. The compound of claim 1, wherein, independently for each occurrence, P comprises an entire sequence selected from LRRNPAD, NPADYRG, NPADYHG, VRLRWNPADYP, LRGNPAD, WNPADYR, LRFNPAD, GSLRYNP, LRVNPADYG, LRRNPADYG, VRLRWNPADYP, RLRVNPADY, LRVNPADYG, WIDVRLRGNPA, RLNPADY, RFNPADY, RLRLNPADY, RLRGNPADY, DVRLRINPADY, DVRLRVNPADY, WVDYNLKWNPDDY, YNLKWNPDDY, KWNPDDY, LFSHLQNEQWVDY, NEQWVDY and HLQNEQWVDY.

4. The compound of claim 1, wherein at least one occurrence of P is a circularized peptide.

5. The compound of claim 1, wherein, independently for each occurrence, P is Pₐ or P_{b},
wherein Pₐ comprises at least 6 consecutive amino acids of said amino-acid sequence;
wherein P_{b} comprises at least 6 consecutive amino acids of said amino-acid sequence; and wherein
the first peptide n-mer is Pₐ—S—Pₐ and the second peptide n-mer is Pₐ—S—Pₐ—,
the first peptide n-mer is Pₐ—S—Pₐ and the second peptide n-mer is P_{b}—S—P_{b}—,
the first peptide n-mer is P_{b}—S—P_{b} and the second peptide n-mer is P_{b}—S—P_{b}—,
the first peptide n-mer is Pₐ—S—P_{b} and the second peptide n-mer is Pₐ—S—P_{b}—,
the first peptide n-mer is Pₐ—S—P_{b} and the second peptide n-mer is Pₐ—S—Pₐ—, or
the first peptide n-mer is Pₐ—S—P_{b} and the second peptide n-mer is P_{b}—S—P_{b}.

6. The compound of claim 5, wherein the biopolymer scaffold is selected from the group consisting of albumin, alpha1-globulins, alpha2-globulins, beta-globulins and immunoglobulins.

7. The compound of claim 1, wherein the biopolymer scaffold is human transferrin.

8. The compound of claim 1, wherein the compound is non-immunogenic in humans.

9. A pharmaceutical composition comprising the compound of claim 1 and at least one pharmaceutically acceptable excipient.

10. The pharmaceutical composition of claim 9 for use in therapy.

11. The pharmaceutical composition for use according to claim 10, for use in prevention or treatment of myasthenia gravis (MG), or of an autoimmune channelopathy such as autoimmune autonomic ganglionopathy or Morvan syndrome, or of a paraneoplastic neurological syndrome in an individual.

12. The pharmaceutical composition for use according to claim 11, wherein the composition is administered at a dose of 1-1000 mg compound per kg body weight of the individual.

13. The pharmaceutical composition for use according to claim 11, wherein the composition is administered subcutaneously, intramuscularly or intravenously.

14. A method of sequestering one or more antibodies present in an individual, comprising:
obtaining a pharmaceutical composition as defined in claim 9, wherein the composition is non-immunogenic in the individual and wherein the one or more antibodies present in the individual are specific for at least one occurrence of P, or for peptide Pₐ and/or peptide P_{b}; and
administering the pharmaceutical composition to the individual.

15. A method of ameliorating or treating MG or an autoimmune channelopathy such as autoimmune autonomic ganglionopathy or Morvan syndrome, or a paraneoplastic neurological syndrome in an individual in need thereof, comprising:
obtaining a pharmaceutical composition as defined in claim 9; and
administering an effective amount of the pharmaceutical composition to the individual.
